SEQUENCE LISTING (73 sequences)

SEQ ID NO: 1 - protein, 1031 aa, synthetic construct
  REGION 1..1031: note = CD19-co-CD20-CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 2 - protein, 488 aa, synthetic construct
  REGION 1..488: note = CD19 BM CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 1110 aa, synthetic construct
  REGION 1..1110: note = CLL1-co-CD33 CAR
MALPVTALLLPLALLLHAARPQVQLVESGGGSVQAGGALSLSCAASGYTVRIDYMGWYRQTPGKGREPVATIASNGGTAYADSVEGRFTISQDNAKNSVYLQMNTLKPGDTAMYYCAAGTWPTLTYFGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLVESGGGSVQAGGSLRLSCAASGYTYSINCMGWFRQAPGKEREGVAVISTGGGRTDYRDSVKGRFTISQDNAKNTVYLQMNSLKPEDTAMYYCAGKTTYPGYGCGLGRSAYNYWGQGTQVTVSSLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSGSGATNFSLLKQAGDVEENPGPMALPVTALLLPLALLLHAARPQVQLVESGGGLVQAGGSLRLSCAASGNVFRFNIMGWYRQAPGNQRELVASIDDGGDRSYADSVEGRFTISRENGKKIMYLQMNSLKPEDTAVYYCAAGLGTYLNGRVSMATNYWGQGTQVTVSSLSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 4 - protein, 356 aa, synthetic construct
  REGION 1..356: note = CLL1 BM CAR
MALPVTALLLPLALLLHAARPQVQLVESGGGSVQAGGALSLSCAASGYTVRIDYMGWYRQTPGKGREPVATIASNGGTAYADSVEGRFTISQDNAKNSVYLQMNTLKPGDTAMYYCAAGTWPTLTYFGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 21 aa, synthetic construct
  REGION 1..21: note = Signal Peptide
MALPVTALLLPLALLLHAARP

SEQ ID NO: 6 - protein, 242 aa, synthetic construct
  REGION 1..242: note = CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 7 - protein, 45 aa, synthetic construct
  REGION 1..45: note = CD8 hinge
TTTPAPRPPTPAPTIASQPLSLRPEACRPAAGGAVHTRGLDFACD

SEQ ID NO: 8 - protein, 24 aa, synthetic construct
  REGION 1..24: note = CD8 TM
IYIWAPLAGTCGVLLLSLVITLYC

SEQ ID NO: 9 - protein, 42 aa, synthetic construct
  REGION 1..42: note = 4-1BB (CD137) co-stimulatory signaling domain
KRGRKKLLYIFKQPFMRPVQTTQEEDGCSCRFPEEEEGGCEL

SEQ ID NO: 10 - protein, 112 aa, synthetic construct
  REGION 1..112: note = CD3 primary intracellular signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 249 aa, synthetic construct
  REGION 1..249: note = CD20 scFv
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 12 - protein, 21 aa, synthetic construct
  REGION 1..21: note = TLR4 TM
TIIGVSVLSVLVVSVVAVLVY

SEQ ID NO: 13 - protein, 60 aa, synthetic construct
  REGION 1..60: note = TLR4 primary intracellular signaling domain
KFYFHLMLLAGCIKYGRGENIYDAFVIYSSQDEDWVRNELVKNLEEGVPPFQLCLHYRDF

SEQ ID NO: 14 - protein, 116 aa, synthetic construct
  REGION 1..116: note = CLL1 sdAb
QVQLVESGGGSVQAGGALSLSCAASGYTVRIDYMGWYRQTPGKGREPVATIASNGGTAYADSVEGRFTISQDNAKNSVYLQMNTLKPGDTAMYYCAAGTWPTLTYFGQGTQVTVSS

SEQ ID NO: 15 - protein, 39 aa, synthetic construct
  REGION 1..39: note = CD28 hinge
IEVMYPPPYLDNEKSNGTIIHVKGKHLCPSPLFPGPSKP

SEQ ID NO: 16 - protein, 27 aa, synthetic construct
  REGION 1..27: note = CD28 TM
FWVLVVVGGVLACYSLLVTVAFIIFWV

SEQ ID NO: 17 - protein, 41 aa, synthetic construct
  REGION 1..41: note = CD28 primary intracellular signaling domain
RSKRSRLLHSDYMNMTPRRPGPTRKHYQPYAPPRDFAAYRS

SEQ ID NO: 18 - protein, 124 aa, synthetic construct
  REGION 1..124: note = CD33 C2 domain sdAb
QVQLVESGGGLVQAGGSLRLSCAASGNVFRFNIMGWYRQAPGNQRELVASIDDGGDRSYADSVEGRFTISRENGKKIMYLQMNSLKPEDTAVYYCAAGLGTYLNGRVSMATNYWGQGTQVTVSS

SEQ ID NO: 19 - protein, 21 aa, synthetic construct
  REGION 1..21: note = TLR1 TM
LLIVTIVATMLVLAVTVTSLC

SEQ ID NO: 20 - protein, 185 aa, synthetic construct
  REGION 1..185: note = TLR1 primary intracellular signaling domain
SYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 21 - protein, 127 aa, synthetic construct
  REGION 1..127: note = CD33 V domain sdAb
EVQLVESGGGSVQAGGSLRLSCAASGYTYSINCMGWFRQAPGKEREGVAVISTGGGRTDYRDSVKGRFTISQDNAKNTVYLQMNSLKPEDTAMYYCAGKTTYPGYGCGLGRSAYNYWGQGTQVTVSS

SEQ ID NO: 22 - protein, 21 aa, synthetic construct
  REGION 1..21: note = TLR2 TM
ALVSGMCCALFLLILLTGVLC

SEQ ID NO: 23 - protein, 60 aa, synthetic construct
  REGION 1..60: note = TLR2 primary intracellular signaling domain
HRFHGLWYMKMMWAWLQAKRKPRKAPSRNICYDAFVSYSERDAYWVENLMVQELENFNPP

SEQ ID NO: 24 - protein, 22 aa, synthetic construct
  REGION 1..22: note = P2A
GSGATNFSLLKQAGDVEENPGP

SEQ ID NO: 25 - protein, 20 aa, synthetic construct
  REGION 1..20: note = GS linker 1
GGGGSGGGGSGGGGSGGGGS

SEQ ID NO: 26 - protein, 5 aa, synthetic construct
  REGION 1..5: note = GS linker 2
GGGGS

SEQ ID NO: 27 - skipped

SEQ ID NO: 28 - protein, 5 aa, synthetic construct
  REGION 1..5: note = GS linker 4
  REPEAT 1..5: note = can be presented in repeats of any integer
GSGGS

SEQ ID NO: 29 - protein, 4 aa, synthetic construct
  REGION 1..4: note = GS linker 5
  REPEAT 1..4: note = can be presented in repeats of any integer
GGGS

SEQ ID NO: 30 - protein, 786 aa, synthetic construct
  REGION 1..786: note = TLR1
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSNLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 31 - protein, 784 aa, synthetic construct
  REGION 1..784: note = TLR2
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 32 - protein, 904 aa, synthetic construct
  REGION 1..904: note = TLR3
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 33 - protein, 839 aa, synthetic construct
  REGION 1..839: note = TLR4
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 34 - protein, 858 aa, synthetic construct [sequence omitted]

SEQ ID NO: 35 - protein, 796 aa, synthetic construct
  REGION 1..796: note = TLR6
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGSTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 36 - protein, 1049 aa, synthetic construct
  REGION 1..1049: note = TLR7
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 37 - protein, 981 aa, synthetic construct
  REGION 1..981: note = TLR8
VGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 38 - protein, 1032 aa, synthetic construct
  REGION 1..1032: note = TLR9
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 39 - protein, 811 aa, synthetic construct
  REGION 1..811: note = TLR10
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGTRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

SEQ ID NO: 40 - protein, 208 aa, synthetic construct
  REGION 1..208: note = Exemplary TLR polypeptide 1 (TLR4)
TIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 41 - protein, 205 aa, synthetic construct
  REGION 1..205: note = Exemplary TLR polypeptide 2 (TLR2)
LSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 42 - protein, 212 aa, synthetic construct
  REGION 1..212: note = Exemplary TLR polypeptide 3 (TLR1)
LSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 43 - protein, 492 aa, synthetic construct
  REGION 1..492: note = CLL1/CD33 dual CAR
MALPVTALLLPLALLLHAARPQVQLVESGGGSVQAGGALSLSCAASGYTVRIDYMGWYRQTPGKGREPVATIASNGGTAYADSVEGRFTISQDNAKNSVYLQMNTLKPGDTAMYYCAAGTWPTLTYFGQGTQVTVSSGGGGSEVQLVESGGGSVQAGGSLRLSCAASGYTYSINCMGWFRQAPGKEREGVAVISTGGGRTDYRDSVKGRFTISQDNAKNTVYLQMNSLKPEDTAMYYCAGKTTYPGYGCGLGRSAYNYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 241 aa, synthetic construct
  REGION 1..241: note = Tandem anti-BCMA sdAb
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSS

SEQ ID NO: 45 - protein, 17 aa, synthetic construct
  REGION 1..17: note = GM-CSF signal peptide
MWLQSLLLLGTVACSIS

SEQ ID NO: 46 - protein, 29 aa, synthetic construct
  REGION 1..29: note = IL-15 signal peptide
MRISKPHLRSISIQCYLCLLLNSHFLTEA

SEQ ID NO: 47 - protein, 8 aa, synthetic construct
  REGION 1..8: note = FLAG tag
DYKDDDDK

SEQ ID NO: 48 - protein, 9 aa, synthetic construct
  REGION 1..9: note = HA tag
YPYDVPDYA

SEQ ID NO: 49 - protein, 10 aa, synthetic construct
  REGION 1..10: note = c-Myc tag
EQKLISEEDL

SEQ ID NO: 50 - protein, 83 aa, synthetic construct
  REGION 1..83: note = CD28 4-1BB intracellular signaling domain
RSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 51 - protein, 136 aa, synthetic construct
  REGION 1..136: note = NKG2D 81-216
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 52 - protein, 128 aa, synthetic construct
  REGION 1..128: note = NKG2D 89-216
IPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 53 - protein, 119 aa, synthetic construct
  REGION 1..119: note = NKG2D 98-216
PCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 54 - protein, 216 aa, synthetic construct
  REGION 1..216: note = NKG2D 1-216
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 55 - protein, 243 aa, synthetic construct
  REGION 1..243: note = GPC3 scFv
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 56 - protein, 495 aa, synthetic construct
  REGION 1..495: note = Anti-GPC3 4-1BB CAR
MALPVTALLLPLALLLHAARPDYKDDDDKDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 882 aa, synthetic construct
  REGION 1..882: note = Anti-GPC3 4-1BB CAR armored with NKG2D TLR4 chimeric receptor
MALPVTALLLPLALLLHAARPDYKDDDDKDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 58 - protein, 927 aa, synthetic construct
  REGION 1..927: note = Anti-GPC3 4-1BB CAR armored with NKG2D CD8 TLR4 chimeric receptor
MALPVTALLLPLALLLHAARPDYKDDDDKDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 59 - protein, 873 aa, synthetic construct
  REGION 1..873: note = Anti-CD19 4-1BB CAR armored with NKG2D TLR4 chimeric receptor
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 60 - protein, 918 aa, synthetic construct
  REGION 1..918: note = Anti-CD19 4-1BB CAR armored with NKG2D CD8 TLR4 chimeric receptor
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 61 - protein, 985 aa, synthetic construct
  REGION 1..985: note = Tandem anti-BCMA-co-anti-BCMA CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 62 - protein, 737 aa, synthetic construct
  REGION 1..737: note = Single anti-BCMA-co-anti-BCMA CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 63 - protein, 778 aa, synthetic construct
  REGION 1..778: note = Single anti-BCMA-co-anti-BCMA-CD8 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 64 - protein, 772 aa, synthetic construct
  REGION 1..772: note = Single anti-BCMA-co-anti-BCMA-CD28 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSIEVMYPPPYLDNEKSNGTIIHVKGKHLCPSPLFPGPSKPTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 65 - protein, 986 aa, synthetic construct
  REGION 1..986: note = Tandem anti-BCMA-co-anti-GPRC5D CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPQSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTVTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 66 - protein, 487 aa, synthetic construct
  REGION 1..487: note = Tandem anti-BCMA CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 363 aa, synthetic construct
  REGION 1..363: note = Single anti-BCMA CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 119 aa, synthetic construct
  REGION 1..119: note = First single anti-BCMA sdAb
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSS

SEQ ID NO: 69 - protein, 117 aa, synthetic construct
  REGION 1..117: note = Second single anti-BCMA sdAb
QVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSS

SEQ ID NO: 70 - protein, 248 aa, synthetic construct
  REGION 1..248: note = Anti-GPRC5D scFv
QSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTV

SEQ ID NO: 71 - protein, 742 aa, synthetic construct
  REGION 1..742: note = CLL1-co-CD33-2 CAR
MALPVTALLLPLALLLHAARPQVQLVESGGGSVQAGGALSLSCAASGYTVRIDYMGWYRQTPGKGREPVATIASNGGTAYADSVEGRFTISQDNAKNSVYLQMNTLKPGDTAMYYCAAGTWPTLTYFGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLVESGGGSVQAGGSLRLSCAASGYTYSINCMGWFRQAPGKEREGVAVISTGGGRTDYRDSVKGRFTISQDNAKNTVYLQMNSLKPEDTAMYYCAGKTTYPGYGCGLGRSAYNYWGQGTQVTVSSQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 72 - protein, 1032 aa, synthetic construct
  REGION 1..1032: note = Tandem anti-BCMA-co-anti-GPRC5D-CD8 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPQSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTVTTTPAPRPPTPAPTIASQPLSLRPEACRPAAGGAVHTRGLDFACDTTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 73 - protein, 1025 aa, synthetic construct
  REGION 1..1025: note = Tandem anti-BCMA-co-anti-GPRC5D-CD28 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPQSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTVIEVMYPPPYLDNEKSNGTIIHVKGKHLCPSPLFPGPSKPTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

(19) United States
(12) Patent Application Publication
(10) Pub. No.: US 2024/0350545 A1
(43) Pub. Date: Oct. 24, 2024

(54) MODIFIED IMMUNE CELLS EXPRESSING TLR RECEPTORS

(71) Applicant: LEGEND BIOTECH IRELAND LIMITED, Dublin 18 (IE)

(72) Inventors: Yanliang ZHU, Nanjing (CN); Qingling JIANG, Nanjing (CN); Zhongyuan TU, Nanjing (CN); Yafeng ZHANG, Nanjing (CN); Shu WU, Nanjing (CN); Xiaohu FAN, Nanjing (CN)

(21) Appl. No.: 18/683,208

(22) PCT Filed: Aug. 18, 2022
(86) PCT No.: PCT/CN2022/113170

§ 371 Date: Feb. 12, 2024

(30) Foreign Application Priority Data
Aug. 18, 2021 (WO) PCT/CN2021/113239
Sep. 30, 2021 (WO) PCT/CN2021/122129
Nov. 25, 2021 (WO) PCT/CN2021/133061
Aug. 15, 2022 (WO) PCT/CN2022/112578

(51) Int. Cl. A61K 35/17; A61K 39/00; A61P 35/00; C07K 14/705; C07K 14/725; C07K 16/28; C07K 16/30; C12N 5/0783; C12N 15/86

(52) U.S. Cl. CPC A61K 35/17; A61K 39/4611; A61K 39/4613; A61K 39/4631; A61K 39/4632; A61K 39/464412; A61K 39/464417; A61K 39/464424; A61K 39/464429; A61K 39/464474; A61P 35/00; C07K 14/7051; C07K 14/70596; C07K 16/2803; C07K 16/2851; C07K 16/2863; C07K 16/2887; C07K 16/303; C12N 5/0638; C12N 5/0646; C12N 15/86; A61K 2239/13; C07K 2317/569; C07K 2317/622; C07K 2319/02; C07K 2319/03; C12N 2510/00; C12N 2740/15043

(57) ABSTRACT

The present application provides modified immune cells that express TLR receptors. In some embodiments, the modified immune cell further comprises an engineered receptor such as a chimeric antigen receptor (CAR). The present application also provides methods and pharmaceutical compositions for cancer treatment using the modified immune cells described herein.

### CROSS REFERENCE TO RELATED APPLICATION

This application claims priority benefits of International Application No. PCT/CN2021/113239, filed on Aug. 18, 2021, International Application No. PCT/CN2021/122129, filed on Sep. 30, 2021, International Application No. PCT/CN2021/133061, filed on Nov. 25, 2021 and International Application No. PCT/CN2022/112578, filed on Aug. 15, 2022, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: P11234-PCT.220817.Sequence listing.xml, date recorded: Aug. 17, 2022, size: 117 KB).

### FIELD

The present application relates to modified immune cells that express TLR receptors, and methods of use thereof for treating a disease or condition such as cancer.

### BACKGROUND

Chimeric antigen receptor (CAR) T cells are cells that have been modified to produce an engineered T cell receptor in order to elicit an immune response. For example, CAR-T cells may be designed to more effectively recognize cancer cells for improved cancer therapy. Despite the success of CAR-T therapies, these methods often suffer from poor in vivo T cell expansion and exhaustion, leading to decreased durability of clinical remissions in patients with hematologic malignancies (e.g., acute myeloid leukemia). There remains a need for highly efficient cell-based cancer immunotherapy.

NKG2D is a transmembrane protein belonging to the NKG2 family of C-type lectin-like receptors. In humans, it is expressed by NK cells, γδ T cells and CD8+ αβ T cells. NKG2D ligands are induced-self proteins from MIC and RAET1/ULBP families which are completely absent or present only at low levels on surface of normal cells, but they are overexpressed by infected, transformed, senescent and stressed cells (Zingoni, A, et al., 2018, Front Immunol. 9:476).

Toll like receptors (TLRs) are pattern recognition receptors that detect invading pathogens and activate the innate and adaptive immune responses. TLRs serve as potent co-stimulatory molecules on T cells, and are expressed on the cell surface of activated T cells (e.g., memory CD4+ and CD8+ T cells). Therefore, the activation of TLRs on T cells directly enhances T cell receptor (TCR) signal-induced T cell activation, function, and survival (Gelman, A E, et al., 2004, 172(10): 6065-6073). Given their crucial role in the immune system, TLR activation has been employed to strengthen immune responses. Conversely, inhibitors of TLR activation can diminish autoimmune and other undesirable immune responses (Lu, H, 2014, Front. in Immunol. 5:83). Studies have shown that engineered immune cells, such as T cells, expressing CARs can be armored with toll/interleukin-1 (IL-1) receptor (TIR) domains in order to provide enhanced anti-tumor activity. See, for example Manavalan, B, et al., 2011, Front. Physiol. 2:41.

The disclosures of all publications, patents, patent applications and published patent applications referred to herein are hereby incorporated herein by reference in their entirety.

### BRIEF SUMMARY

The present application provides modified immune cells that express TLR receptors, and methods of use thereof for treating a disease or condition, such as cancer.

One aspect of the present application provides a modified immune cell comprising: a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain; wherein upon binding of the first target binding domain and second target binding domain to their corresponding target, the first TLR signaling domain and the second TLR signaling domain associate with each other to form a TLR signaling moiety capable of inducing TLR signaling.

In some embodiments according to the modified immune cell, the first target binding domain and the second target binding domain each binds to a subunit of a multimeric target molecule. In some embodiments, the subunits of the multimeric target molecule are the same. In some embodiments, the subunits of the multimeric target molecule are different. In some embodiments, the first target binding domain and the second binding domain bind to the same target molecule. In some embodiments, the first target binding domain and the second binding domain each binds to the same target site on the target molecule. In some embodiments, the first target binding domain and the second target binding domain are the same.

Another aspect of the present application provides a modified immune cell according to the modified immune cells wherein the first target binding domain and the second binding domain bind to the same target molecule described above, wherein the first target binding domain and the second target binding domain each binds to a different non-overlapping target sites on a single target molecule.

In some embodiments according to any of one the modified immune cells described above, the first TLR transmembrane domain and the first TLR signaling domain are derived from the same TLR molecule. In some embodiments, the second TLR transmembrane domain and the second TLR signaling domain are derived from the same TLR molecule. In some embodiments, the first TLR transmembrane domain and the second TLR transmembrane domain are the same. In some embodiments, the first TLR signaling domain and the second TLR signaling domain are the same. In some embodiments, the first TLR transmembrane domain and/or first TLR signaling domain are derived from TLR4. In some embodiments, the first TLR transmembrane domain and the second TLR transmembrane domain are different. In some embodiments, the first TLR signaling domain and the second TLR signaling domain are different. In some embodiments, the first TLR transmembrane domain and/or first TLR signaling domain are derived from TLR2. In some embodiments, the second TLR transmembrane domain and/or first TLR signaling domain are derived from TLR6. In some embodiments, the second TLR transmembrane domain and/or second TLR signaling domain are derived from TLR1.

In some embodiments according to any of one the modified immune cells described above, the first target binding domain and/or the second target binding domain is an antibody moiety or antigen-binding fragment thereof. In some embodiments, the first target binding domain and/or second target binding domain is a scFv or sdAb. In some embodiments, the scFv or sdAb that specifically binds to CD33, CLL1, CD123, CD19, CD20, CD22, BCMA, GPRC5D, and GPC3.

In some embodiments according to any of one the modified immune cells described above, the target molecule is an immune checkpoint protein. In some embodiments, the target molecule is selected from the group consisting of PD-1, CD70, CD27, SIRPα, and TIGIT.

In some embodiments according to any of one the modified immune cells described above, the target molecule is a natural protein expressed on immune cells. In some embodiments, the target molecule is NKG2D. In some embodiments, the target molecule is mutated NKG2D. In some embodiments, the mutated NKG2D comprises a truncated sequence and/or an amino acid substitution, mutation, addition, and/or deletion.

In some embodiments, the target molecule is an extracellular antigen binding domain of NKG2D. In some embodiments, the target molecule is full-length sequence of NKG2D.

In some embodiments according to any of one the modified immune cells described above, the modified immune cell is selected from the group consisting of a cytotoxic T cell, a helper T cell, a natural killer (NK) cell, an NK-cell, an iNK-T cell, an NK-T like cell, an αβT cell and a γδT cell. In some embodiments, the modified immune cell is an NK cell. In some embodiments, the modified immune cell is a cytotoxic T cell. In some embodiments, the modified immune cell comprises an engineered receptor. In some embodiments, the engineered receptor is a chimeric antigen receptor (CAR). In some embodiments, the engineered receptor is a modified T-cell receptor (TCR). In some embodiments, the engineered receptor is a T-cell antigen coupler (TAC) receptor.

An additional aspect of the present application provides a modified immune cell according to any of one the modified immune cells described above, wherein the engineered receptor comprises an extracellular domain specifically recognizing the same target molecule as the first polypeptide and/or the second polypeptide. In some embodiments, the engineered receptor comprises an extracellular domain specifically recognizing a non-overlapping target site on the same target molecule as the first polypeptide and/or the second polypeptide. In some embodiments, the engineered receptor comprises an extracellular domain specifically recognizing any of CD19, CLL1, BCMA, and GPC3.

In some embodiments according to any of one the modified immune cells described above, the modified immune cell comprises a first nucleic acid encoding the first polypeptide and a second nucleic acid encoding the second polypeptide.

In some embodiments according to any of one the modified immune cells described above, the first polypeptide and the second polypeptide are the same, and the modified immune cell comprises a first nucleic acid encoding the first polypeptide and the second polypeptide.

In some embodiments according to any of one the modified immune cells described above, the modified immune cell comprises a third nucleic acid encoding the engineered receptor. In some embodiments, the first nucleic acid and the second nucleic acid are operably linked to the same promoter. In some embodiments, the first nucleic acid and the second nucleic acid are operably linked to separate promoters. In some embodiments, the first nucleic acid and the third nucleic acid are operably linked to the same promoter. In some embodiments, the first nucleic acid and the third nucleic acid are operably linked to separate promoters. In some embodiments, the first nucleic acid, the second nucleic acid, and the third nucleic acid are operably linked to the same promoter.

In some embodiments according to any of one the modified immune cells described above, the first target binding domain and second target binding domain specifically recognize a subunit of CD20. In some embodiments, the first target binding domain and second target binding domain specifically recognize a subunit of CD33. In some embodiments, the first target binding domain specifically recognizes the V subunit of CD33, and the second target binding domain specifically recognizes the C2 subunit of CD33. In some embodiments, the first target binding domain and second target binding domain specifically recognize a subunit of BCMA. In some embodiments, the first target binding domain and second target binding domain specifically recognize a subunit of GPRC5D.

In some embodiments according to any of one the modified immune cells described above, the first polypeptide further comprises a first intracellular domain of a first cytokine receptor. In some embodiments, the second polypeptide further comprises a second intracellular domain of a second cytokine receptor. In some embodiments, the first polypeptide further comprises a first intracellular domain of a first cytokine receptor and the second polypeptide further comprises a second intracellular domain of a second cytokine receptor. In some embodiments, the first intracellular domain and the second intracellular domain are the same. In some embodiments, the first intracellular domain and the second intracellular domain are different. In some embodiments, the first cytokine receptor is selected from the group consisting of a GM-CSF receptor, an IL-18 receptor, an IL-21 receptor, an IL-15 receptor, and an IL-23 receptor. In some embodiments, the second cytokine receptor is selected from the group consisting of a GM-CSF receptor, an IL-18 receptor, an IL-21 receptor, an IL-15 receptor, and an IL-23 receptor. In some embodiments, the first intracellular domain of the first cytokine receptor comprise an immunoreceptor tyrosine-based activation motif (ITAM). In some embodiments, the second intracellular domain of the second cytokine receptor comprise an immunoreceptor tyrosine-based activation motif (ITAM). In some embodiments, the first intracellular domain of the first cytokine receptor and the second intracellular domain of the second cytokine receptor comprise an immunoreceptor tyrosine-based activation motif (ITAM). In some embodiments, the C-terminus of the first intracellular domain of the first cytokine receptor is fused to the N-terminus of the first TLR signaling domain. In some embodiments, the C-terminus of the second intracellular domain of the second cytokine receptor is fused to the N-terminus of the second TLR signaling domain. In some embodiments, the C-terminus of the first intracellular domain of the first cytokine receptor is fused to the N-terminus of the first TLR signaling domain; and, the C-terminus of the second intracellular domain of the second cytokine receptor is fused to the N-terminus of the second TLR signaling domain. In some embodiments, the N-terminus of the first intracellular domain of the first cytokine receptor is fused to the C-terminus of the TLR signaling domain. In some embodiments, the N-terminus of the second intracellular domain of the second cytokine receptor is fused to the C-terminus of the TLR signaling domain. In some embodiments, the N-terminus of the first intracellular domain of the first cytokine receptor is fused to the C-terminus of the TLR signaling domain; and, the N-terminus of the second intracellular domain of the second cytokine receptor is fused to the C-terminus of the TLR signaling domain.

One aspect of the present application provides a method of producing a modified immune cell, comprising: introducing into a precursor immune cell a first nucleic acid encoding the first polypeptide and optionally a second nucleic acid encoding the second polypeptide.

In some embodiments according to any one of the methods of production described above, the precursor immune cell is selected from the group consisting of a cytotoxic T cell, a helper T cell, an NK cell, an NK-T cell, an iNK-T cell, an NK-T like cell, an αβT cell and a γδT cell. In some embodiments, the precursor immune cell comprises an engineered receptor. In some embodiments, the method of producing further comprising introducing into the precursor immune cell a third nucleic acid encoding an engineered receptor. In some embodiments, the engineered receptor is a chimeric antigen receptor (CAR), a modified T-cell receptor (TCR), or a T-cell antigen coupler (TAC) receptor.

In some embodiments according to any one of the methods of production described above, the first nucleic acid, the second nucleic acid, and/or the third nucleic acid are on the same vector. In some embodiments, the vector is a viral vector. In some embodiments, the viral vector is selected from the group consisting of an adenoviral vector, an adeno-associated virus vector, a retroviral vector, a lentiviral vector, a herpes simplex viral vector, and derivatives thereof.

In some embodiments according to any one of the methods of production described above, the method further comprises isolating or enriching immune cells comprising the first nucleic acid, the second nucleic acid, and/or the third nucleic acid.

Also provided is a modified immune cell produced by the method according to any one of the methods of production described above.

Further provided is a pharmaceutical composition comprising the modified immune cell according to any one of the modified immune cells described above, and a pharmaceutically acceptable carrier.

Another aspect of the present application provides a method of treating a disease in an individual, comprising administering to the individual an effective amount of the pharmaceutical composition according to any one of the pharmaceutical compositions described above. In some embodiments, the disease is cancer. In some embodiments, the individual is human.

Compositions, uses, kits and articles of manufacture comprising any one of the modified immune cells are also provided.

### BRIEF DESCRIPTION OF THE DRAWINGS

FIG. 1 shows the bicistron expression design of a CAR fusion construct comprising an anti-CD19-CAR and anti-CD20 TLR polypeptides (CD19-co-CD20 CAR; SEQ ID NO: 1). The sequence of the CAR backbone is according to the following pattern from the N-terminus to the C-terminus: a signal peptide (SEQ ID NO: 5), anti-CD19 scFv (SEQ ID NO: 6), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), which is connected to an inducible costimulatory molecule comprising from the N-terminus to the C-terminus: a signal peptide (SEQ ID NO: 5), an anti-CD20 scFv (SEQ ID NO: 11), a TLR4 transmembrane (TM) region (SEQ ID NO: 12), and the cytoplasmic portion of TLR4 (e.g., TLR4 primary intracellular signaling domain) (SEQ ID NO: 13), via a P2A cleavage site (SEQ ID NO: 24).

FIG. 2 shows in vitro cytotoxic effects of CD19 BM CAR-T and CD19-co-CD20 CAR-T cells armored with TLR4. In particular, FIG. 2 shows that CD19 BM CAR-T and CD19-co-CD20 CAR T cells induce Raji target cell lysis in a dose dependent manner in vitro. Untransduced T cells (i.e., “unT”) served as controls in this experiment.

FIG. 3 shows the in vitro IFNγ cytokine section of CD19 BM CAR-T and CD19-co-CD20 CAR-T cells armored with TLR4, co-cultured with Raji target cells. Untransduced T cells (i.e., “unT”) served as controls in this experiment.

FIG. 4 shows the in vitro TNFα cytokine section of CD19 BM CAR-T and CD19-co-CD20 CAR-T cells armored with TLR4, co-cultured with Raji target cells. Untransduced T cells (i.e., “unT”) served as controls in this experiment.

FIG. 5 shows the in vitro killing efficacy CD19 BM CAR-T and CD19-co-CD20 CAR-T cells armored with TLR4 in a repeated tumor stimulation assay with Raji target cells. Untransduced T cells (i.e., “UNT”) served as controls in this experiment.

FIG. 6 shows the in vitro proliferation (e.g., expansion fold) of CD19 BM CAR-T and CD19-co-CD20 CAR-T cells armored with TLR4, after incubation with Raji target cells.

FIG. 7 shows the bicistron expression design of a CAR fusion construct comprising an anti-CLL1-CAR and anti-CD33 TLR polypeptides (CLL1-co-CD33 CAR; SEQ ID NO: 3 and CLL1-co-CD33-2 CAR-T; SEQ ID NO: 71).

FIG. 8 shows in vitro cytotoxic effects of CLL1 BM CAR-T, CLL1-co-CD33 CAR-T cells armored with TLR2 and TLR1 and CLL1-co-CD33-2 CAR-T cells armored with TLR4. In particular, FIG. 8 shows that CLL1 BM CAR-T, CLL1-co-CD33 CAR-T and CLL1-co-CD33-2 CAR-T cells induce U937 target cell lysis in a dose dependent manner in vitro. Untransduced T cells (i.e., “unT”) served as controls in this experiment.

FIG. 9 shows the in vitro IFNγ cytokine section of CLL1 BM CAR-T, CLL1-co-CD33 CAR-T cells armored with TLR2 and TLR1 and CLL1-co-CD33-2 CAR-T armored with TLR4, co-cultured with U937 target cells. Untransduced T cells (i.e., “unT”) served as controls in this experiment.

FIG. 10 shows the in vitro TNFα cytokine section of CLL1 BM CAR-T, CLL1-co-CD33 CAR-T cells armored with TLR2 and TLR1 and CLL1-co-CD33-2 CAR-T armored with TLR4, co-cultured with U937 target cells. Untransduced T cells (i.e., “unT”) served as controls in this experiment.

FIG. 11 shows the in vitro killing efficacy CLL1 BM CAR-T, CLL1-co-CD33 CAR-T cells armored with TLR2 and TLR1 and CLL1-co-CD33-2 CAR-T armored with TLR4 in a repeated tumor stimulation assay with U937 target cells. Untransduced T cells (i.e., “UNT”) served as controls in this experiment.

FIG. 12 shows the in vitro proliferation (e.g., expansion fold) of CLL1 BM CAR-T, CLL1-co-CD33 CAR-T cells armored with TLR2 and TLR1 and CLL1-co-CD33-2 CAR-T armored with TLR4, after incubation with U937 target cells.

FIG. 13 shows a schematic of an in vivo efficacy study of exemplary CLL1-co-CD33 CAR αβT and CLL1-co-CD33 CAR γδT treatment in a U937-Luc xenograft mouse model.

FIG. 14 shows the in vivo efficacy of CLL1-co-CD33 CAR αβT cells in a U937-Luc xenograft mouse model.

FIG. 15 shows the in vivo efficacy of CLL1-co-CD33 CAR γδT cells and CLL1-co-CD33-2 CAR γδT cells in a U937-Luc xenograft mouse model.

FIGS. 16A-16B show exemplary constructs of CAR or TCRs armored with NKG2D or mutated NKG2D TLR chimeric receptor. FIG. 16A shows a schematic of a CAR armored with NKG2D or mutated NKG2D TLR chimeric receptor. FIG. 16B shows a schematic of a TCR armored with NKG2D or mutated NKG2D TLR chimeric receptor.

FIGS. 17A-17B show exemplary constructs of CAR or TCRs armored with chimeric receptor which includes a binding domain targeting NKG2D ligands, TLR transmembrane domain and intracellular effector domain. FIG. 17A shows a schematic of a CAR armored with chimeric receptor which includes a binding domain targeting NKG2D ligands, TLR transmembrane domain and intracellular effector domain. FIG. 17B shows a schematic of a TCR armored with chimeric receptor which includes a binding domain targeting NKG2D ligands, TLR transmembrane domain and intracellular effector domain.

FIGS. 18A-18B show exemplary constructs of CAR or TCRs armored with NKG2D or mutated NKG2D TLR4 chimeric receptor. FIG. 18A shows a second generation CAR armored with NKG2D or mutated NKG2D TLR4 chimeric receptor. FIG. 18B shows TCR armored with NKG2D or mutated NKG2D TLR4 chimeric receptor.

FIG. 19 shows the in vitro killing efficacy anti-GPC3 CAR-T and anti-GPC3 CAR-T cells armored with NKG2D-TLR4 or NKG2D-CD8-TLR4 chimeric receptor in a repeated tumor stimulation assay with Huh7 target cells. Untransduced T cells (i.e., “UNT”) served as controls in this experiment.

FIG. 20 shows the in vitro proliferation of anti-GPC3 CAR-T and anti-GPC3 CAR-T cells armored with NKG2D-TLR4 or NKG2D-CD8-TLR4 chimeric receptor after incubation with Huh7 target cells.

FIG. 21 shows the in vitro IFNγ cytokine section of anti-GPC3 CAR-T and anti-GPC3 CAR-T cells armored with NKG2D-TLR4 or NKG2D-CD8-TLR4 chimeric receptor, co-cultured with Huh7 target cells. Untransduced T cells (i.e., “UNT”) served as controls in this experiment.

FIG. 22 shows the in vitro killing efficacy anti-CD19 CAR-T and anti-CD19 CAR-T cells armored with NKG2D-TLR4 or NKG2D-CD8-TLR4 chimeric receptor in a repeated tumor stimulation assay with Raji target cells. Untransduced T cells (i.e., “UNT”) served as controls in this experiment.

FIG. 23 shows the in vitro proliferation of anti-CD19 CAR-T and anti-CD19 CAR-T cells armored with NKG2D-TLR4 or NKG2D-CD8-TLR4 chimeric receptor after incubation with Raji target cells.

FIG. 24 shows the in vivo efficacy of anti-GPC3 CAR-T cells and anti-GPC3 CAR-T cells armored with NKG2D-CD8-TLR4 chimeric receptor in Huh7 xenograft model.

FIG. 25 shows the bicistron expression design of a CAR fusion construct comprising a tandem anti-BCMA-co-anti-BCMA CAR (SEQ ID NO: 61) or single anti-BCMA-co-anti-BCMA CAR (SEQ ID NO: 62), single anti-BCMA-co-anti-BCMA-CD8 CAR (SEQ ID NO: 63), single anti-BCMA-co-anti-BCMA-CD28 CAR (SEQ ID NO: 64) in addition to tandem anti-BCMA-co-anti-GPRC5D CAR (SEQ ID NO: 65), tandem anti-BCMA-co-anti-GPRC5D-CD8 CAR (SEQ ID NO: 72), tandem anti-BCMA-co-anti-GPRC5D-CD28 CAR (SEQ ID NO: 73).

FIG. 26 shows in vitro cytotoxic effects of anti-BCMA-CAR-γδT and anti-BCMA-co-anti-BCMA-γδT cells armored with TLR4 intracellular signaling. Untransduced γδT cells (i.e., “Un-γδT”) served as controls in this experiment.

FIG. 27 shows the in vitro IFN-γ, TNF-α and GM-CSF cytokine secretion of anti-BCMA-CAR-γδT and anti-BCMA-co-anti-BCMA-γδT cells armored with TLR4 intracellular signalling, co-cultured with BCMA-positive NCI-H929 target tumor cells. Untransduced γδT cells (i.e., “Un-γδT”) served as controls in this experiment.

FIG. 28 shows the in vitro killing efficacy and persistence of tandem anti-BCMA-CAR-γδT, tandem anti-BCMA-co-anti-BCMA-CAR γδT, single anti-BCMA-co-anti-BCMA CAR γδT, single anti-BCMA-co-anti-BCMA-CD8 CAR γδT, single anti-BCMA-co-anti-BCMA-CD28 CAR γδT, tandem anti-BCMA-co-anti-GPRC5D-CAR γδT and tandem anti-BCMA-co-anti-GPRC5D-CD8 CAR-γδT cells in a repeated tumor stimulation assay with BCMA-positive NCI-H929 target tumor cells. Untransduced γδT cells (i.e., “Un-γδT”) served as controls in this experiment.

FIG. 29 shows the persistence of tandem anti-BCMA-CAR-γδT and tandem anti-BCMA-co-anti-BCMA-γδT cells armored with TLR4 intracellular signalling in allogeneic setting with co-incubation of γδT cells, allogeneic PBMCs and BCMA-positive NCI-H929 target tumor cells at a ratio of 1:60:1.

FIG. 30 shows the in vivo killing efficacy and persistence of tandem anti-BCMA-CAR-γδT, tandem anti-BCMA-co-anti-BCMA-γδT cells armored with TLR4 intracellular signalling and tandem anti-BCMA-co-anti-GPRC5D-CD8-γδT cells in a BCMA-positive RPMI-8226 tumor-bearing xenograft model. Untransduced γδT cells (i.e., “Un-γδT”) and vehicle HBSS served as controls in this experiment.

FIGS. 31A-31B show the body weight change and IFN-γ, TNF-α and GM-CSF cytokine secretion of tandem anti-BCMA-CAR-γδT and tandem anti-BCMA-co-anti-BCMA-γδT cells armored with TLR4 intracellular signalling in a BCMA-positive RPMI-8226 tumor-bearing xenograft model. Untransduced γδT cells (i.e., “Un-γδT”) and vehicle HBSS served as controls in this experiment.

### DETAILED DESCRIPTION

The present application provides modified immune cells that comprise Toll-like receptor (TLR) co-stimulatory molecules (e.g., TLR polypeptides), and methods of use thereof for treating cancer. In some embodiments, the modified immune cells comprise a TLR-based multimer comprising a first TLR polypeptide and a second TLR polypeptide, which have potent and long-lasting tumor lytic activity and improved exhaustion profile compared to modified immune cells not expressing said polypeptides. In some embodiments, the first TLR polypeptide comprises: i) a first target binding domain (e.g., an antibody moiety or fragment thereof), ii) a first TLR transmembrane domain, and iii) a first TLR signaling domain; and the second TLR polypeptide comprises: i) a second target binding domain (e.g., an antibody moiety or fragment thereof), ii) a second TLR transmembrane domain, and iii) a second TLR signaling domain. Upon binding of the first target binding domain and second target binding domain to their corresponding targets, the first TLR signaling domain and the second TLR signaling domain associate with each other to form a TLR signaling moiety capable of inducing TLR signaling. In some embodiments, the modified immune cells are T cells.

In some embodiments, the modified cells further express a chimeric antigen receptor (CAR) that specifically recognizes a target antigen of interest. The TLR polypeptides described herein augment CAR-T cell proliferation and enhance anti-tumor activity.

The activation of TLR signaling on the TLR polypeptides is target dependent and initiates by dimerized or multimerized TLR polypeptides polypeptide and formation of a TLR signaling moiety following binding of the TLR polypeptides to the corresponding target molecules. The present application provides multiple strategies for inducing the formation of a TLR signaling moiety. In a first strategy, the first target binding domain of the first polypeptide and the second binding domain of the second polypeptide each binds to a subunit of a multimeric target molecule. Upon binding of the target binding domains to their cognate target subunit, the first TLR signaling domain and the second TLR signaling domain associate to each other to form the TLR signaling moiety. In a second strategy, the first target binding domain of the first polypeptide and the second binding domain of the second polypeptide each binds to a different, non-overlapping target site on a target molecule. Upon binding of the target binding domains to their cognate target molecule, the first TLR signaling domain and the second TLR signaling domain associate to each other to form the TLR signaling moiety. In a third strategy, when the immune cell comprises an engineered receptor comprising an extracellular domain, the extracellular domain and the first or second target binding domain each binds to a different, non-overlapping target side on the same target molecule. Without being bound by theory, it is believed that, upon binding of the extracellular domain of the engineered receptor to the target molecule, an immunological synapse is formed around the engineered receptor. The first and second polypeptides are recruited to the same immunological synapse, which allows the first TLR signaling domain and the second TLR signaling domain to associate to each other, thereby forming the TLR signaling moiety. In yet another strategy, the first target binding domain and the second target binding domain each binds to the same monomeric target molecule (e.g., on the same target site), and the binding of the first target binding and the second target binding domain to the target molecule allows the first TLR signaling domain and the second TLR signaling domain to associate to each other, thereby forming the TLR signaling moiety.

Accordingly, one aspect of the present application provides a modified immune cell (e.g., T cell) comprising a first polypeptide comprising: i) a first target binding domain (e.g., an antibody moiety or fragment thereof), ii) a first TLR transmembrane domain, and iii) a first TLR signaling domain; and b) a second polypeptide comprising: i) a second target binding domain (e.g., an antibody moiety or fragment thereof), ii) a second TLR transmembrane domain, and iii) a second TLR signaling domain, wherein the first target binding domain and the second target binding domain each binds to a subunit of a multimeric target molecule, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the first target binding domain and the second binding domain bind to the same target molecule. In some embodiments, the first target binding domain and the second binding domain each binds to the same target site on the target molecule. In some embodiments, the modified immune cell further comprises an engineered receptor, such as a chimeric antigen receptor, a modified T-cell receptor, or a T-cell antigen coupler (TAC) receptor.

Another aspect of the present application provides a modified immune cell (e.g., T cell) comprising a first polypeptide comprising: i) a first target binding domain (e.g., an antibody moiety or fragment thereof), ii) a first TLR transmembrane domain, and iii) a first TLR signaling domain; and b) a second polypeptide comprising: i) a second target binding domain (e.g., an antibody moiety or fragment thereof), ii) a second TLR transmembrane domain, and iii) a second TLR signaling domain, wherein the first target binding domain and the second binding domain bind to the same target molecule, and wherein the first target binding domain and the second target binding domain each binds to a different non-overlapping target sites on a single target molecule, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the modified immune cell further comprises an engineered receptor, such as a chimeric antigen receptor (CAR), a modified T-cell receptor, or a T-cell antigen coupler (TAC) receptor.

A further aspect of the present application provided a modified immune cell (e.g., T cell) comprising a first polypeptide comprising: i) a first target binding domain (e.g., an antibody moiety or fragment thereof), ii) a first TLR transmembrane domain, and iii) a first TLR signaling domain; b) a second polypeptide comprising: i) a second target binding domain (e.g., an antibody moiety or fragment thereof), ii) a second TLR transmembrane domain, and iii) a second TLR signaling domain; and c) an engineered receptor (e.g., a CAR), wherein the engineered receptor comprises an extracellular domain specifically recognizing the same target molecule as the first polypeptide and/or the second polypeptide, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the engineered receptor comprises an extracellular domain specifically recognizing a non-overlapping target site on the same target molecule as the first polypeptide and/or the second polypeptide.

Also provided are compositions (such as pharmaceutical compositions), kits and articles of manufacture comprising the modified immune cells, and methods of treating a disease or condition (e.g., cancer) using the modified immune cells described herein.

### I. Definitions

As used herein, “treatment” or “treating” is an approach for obtaining beneficial or desired results including clinical results. For purposes of this disclosure, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease (e.g., cancer). The methods of the present application contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of cancer means to defer, hinder, slow, retard, stabilize, and/or postpone development of the disease. This delay can be of varying lengths of time, depending on the history of the disease and/or individual being treated. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “effective amount” used herein refers to an amount of an agent or a combination of agents, sufficient to treat a specified disorder, condition or disease such as to ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other undesired cell proliferation. In some embodiments, an effective amount is an amount sufficient to delay disease development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, an “individual” or a “subject” refers to a mammal, including, but not limited to, human, bovine, horse, feline, canine, rodent, or primate. In some embodiments, the individual is a human.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which a heterologous nucleic acid is transferred or introduced into the host cell. A “transfected” or “transformed” or “transduced” cell is one which has been transfected, transformed or transduced with a heterologous nucleic acid. The cell includes the primary subject cell and its progeny.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Chimeric antigen receptor” or “CAR” as used herein refers to genetically engineered receptors, which graft one or more antigen specificity onto cells, such as T cells. CARs are also known as “artificial T-cell receptors,” “chimeric T-cell receptors,” or “chimeric immune receptors.” In some embodiments, the CAR comprises an extracellular variable domain of an antibody specific for a tumor antigen, and an intracellular signaling domain of a T cell or other receptors, such as one or more co-stimulatory domains. “CAR-T” refers to a T cell that expresses a CAR. As used herein, a “CLL1 CAR” refers to a CAR that specifically recognizes CLL1, a “CD19 CAR” refers to a CAR that specifically recognizes CD19, a “GPC3 CAR” refers to a CAR that specifically recognizes GPC3, and a “BCMA CAR” refers to a CAR that specifically recognizes BCMA.

“T-cell receptor” or “TCR” as used herein refers to an endogenous or modified T-cell receptor comprising an extracellular antigen binding domain that binds to a specific antigenic peptide bound in an MHC molecule. In some embodiments, the TCR comprises a TCRα polypeptide chain and a TCR β polypeptide chain. In some embodiments, the TCR comprises a TCRγ polypeptide chain and a TCR δ polypeptide chain. In some embodiments, the TCR specifically binds a tumor antigen. “TCR-T” refers to a T cell that expresses a recombinant TCR.

“T-cell antigen coupler receptor” or “TAC receptor” as used herein refers to an engineered receptor comprising an extracellular antigen binding domain that binds to a specific antigen and a T-cell receptor (TCR) binding domain, a transmembrane domain, and an intracellular domain of a co-receptor molecule. The TAC receptor co-opts the endogenous TCR of a T cell that expressed the TAC receptor to elicit antigen-specific T-cell response against a target cell.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term antibody includes, but is not limited to, fragments that are capable of binding antigen, such as Fv, single-chain Fv (scFv), Fab, Fab′, and (Fab′)₂. The term antibody includes conventional four-chain antibodies, and single-domain antibodies, such as heavy-chain only antibodies or fragments thereof, e.g., V_{H}H.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “cell” includes the primary subject cell and its progeny.

It is understood that embodiments of the disclosure described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the modified immune cells and methods of treatment described herein are specifically embraced by the present application and are disclosed herein just as if each and every combination was individually and explicitly disclosed. In addition, all subcombinations of the modified immune cells listed in the embodiments describing such variables are also specifically embraced by the present application and are disclosed herein just as if each and every such sub-combination of proteins was individually and explicitly disclosed herein.

### II. Modified Immune Cells

One aspect of the present application provides a modified immune cell comprising a first polypeptide and a second polypeptide, wherein the first polypeptide and the second polypeptide each encode a TLR polypeptide (e.g., TLR co-stimulatory polypeptides) comprising a TLR signaling domain. In some embodiments, upon binding of the TLR polypeptides to their correspond target, the TLR signaling domains of the first and second polypeptide associate with each other to form a TLR signaling moiety, wherein the TLR signaling moiety induces TLR signaling, resulting in strong anti-tumor effects. The first polypeptide and the second polypeptide may be identical or different.

In some embodiments, the modified immune cells comprising the first and second TLR polypeptides have increased T-cell receptor (TCR) signal-induced T cell activation, function, and/or survival, compared to modified immune cells not comprising said TLR polypeptides. In some embodiments, the TLR polypeptides induce an increased tumor cell killing efficacy by the modified immune cell compared to modified immune cells not comprising said TLR polypeptides. In some embodiments, the TLR polypeptides induce increased tumor cell killing efficacy, such as increased by at least about any one of 10%, 20%, 30%, 40%, 50%, 60%, 70%, 80%, 90%, 12 fold, 14 fold, 16 fold, 20 fold, 25 fold, 30 fold, 40 fold or more, compared to a modified immune cell not comprising said TLR polypeptides. In some embodiments, the TLR polypeptides confer long lasting efficacy for the modified immune cell, e.g., such as increased by at least about any one of 10%, 20%, 30%, 40%, 50%, 60%, 70%, 80%, 90%, 2 fold, 5 fold, 10 fold, 20 fold, 50 fold, 100 fold, 200 fold, 500 fold, 1000 fold or more, compared to a modified immune cell not comprising said TLR polypeptides. In some embodiments, the modified immune cell has reduced toxicity in vivo when administered to an individual compared to a modified immune cell that does comprise a TLR polypeptide. In some embodiments, the modified immune cell decrease exhaustion in vivo when administered to an individual compared to a modified immune cell that compared to a modified immune cell that does comprise a TLR polypeptide. In some embodiments, the TLR polypeptide is a co-stimulatory molecule. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the first TLR transmembrane domain and the first TLR signaling domain are derived from the same TLR molecule. In some embodiments, the first TLR transmembrane domain and the first TLR signaling domain are derived from different TLR molecules. In some embodiments, the second TLR transmembrane domain and the second TLR signaling domain are derived from the same TLR molecule. In some embodiments, the second TLR transmembrane domain and the second TLR signaling domain are derived from different TLR molecules. In some embodiments, the first TLR transmembrane domain and the second TLR transmembrane domain are the same. In some embodiments, the first TLR transmembrane domain and the second TLR transmembrane domain are different. In some embodiments, the first TLR signaling domain and the second TLR signaling domain are different. In some embodiments, the TLR molecule(s) is selected from the group consisting of TLR1, TLR2, TLR3, TLR4, TLR5, TLR6, TLR7, TLR8, and TLR9, such as TLR1, TLR2, TLR4 or TLR6. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling, and wherein the first TLR transmembrane domain, the first TLR signaling domain, the second TLR transmembrane domain, and the second TLR signaling domain are derived from TLR4. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling; wherein the first TLR transmembrane domain and the first TLR signaling domain are derived from TLR2, and wherein the second TLR transmembrane domain and the second TLR signaling domain are derived from TLR1, or wherein the first TLR transmembrane domain and the first TLR signaling domain are derived from TLR1, and wherein the second TLR transmembrane domain and the second TLR signaling domain are derived from TLR2. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling; wherein the first TLR transmembrane domain and the first TLR signaling domain are derived from TLR6, and wherein the second TLR transmembrane domain and the second TLR signaling domain are derived from TLR2, or wherein the first TLR transmembrane domain and the first TLR signaling domain are derived from TLR2, and wherein the second TLR transmembrane domain and the second TLR signaling domain are derived from TLR6. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling, and wherein the first target binding domain and/or the second target binding domain is an antibody moiety or antigen-binding fragment thereof. In some embodiments, the first target binding domain and the second target binding domain are antibody moieties or antigen-binding fragment thereof. In some embodiments, the first polypeptide and the second polypeptide do not comprise an extracellular TGFβ-binding domain of a TGFβ (e.g., TGFβR1 and/or TGFβR2). In some embodiments, the first target binding domain and/or second target binding domain is a scFv or sdAb. In some embodiments, the first target binding domain and/or second target binding domain specifically binds to CD33, CLL1, CD123, CD19, CD20, CD22, BCMA, GPRC5D, NKG2D, or GPC3. In some embodiments, the first target binding domain and the second target binding domain are the same. In some embodiments, the first target binding domain and the second target binding domain are different. In some embodiments, the target molecule of the first polypeptide and/or the second polypeptide is an immune checkpoint protein. In some embodiments, the target molecule of the first polypeptide and/or the second polypeptide is selected from the group consisting of PD-1, CD70, CD27, SIRPα, and TIGIT. In some embodiments according to any of one the modified immune cells described above, the target molecule is a natural protein expressed on immune cells. In some embodiments, the target molecule is NKG2D. In some embodiments, the target molecule is mutated NKG2D. In some embodiments, the mutated NKG2D comprises a truncated sequence and/or an amino acid substitution, mutation, addition, and/or deletion. In some embodiments, the target molecule is an extracellular antigen binding domain of NKG2D. In some embodiments, the target molecule is full-length sequence of NKG2D. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling, and wherein the first target binding domain and the second target binding domain each binds to a subunit of a multimeric target molecule. In some embodiments, the first polypeptide and the second polypeptide do not comprise an extracellular TGFβ-binding domain of a TGFβ (e.g., TGFβR1 and/or TGFβR2). In some embodiments, the first target binding domain and/or the second target binding domain is an antibody moiety or antigen-binding fragment thereof. In some embodiments, the subunits of the multimeric target molecule are different. In some embodiments, the first target binding domain specifically recognizes the V subunit of CD33, and the second target binding domain specifically recognizes the C2 subunit of CD33. In some embodiments, the subunits of the multimeric target molecule are the same. In some embodiments, the first target binding domain and second target binding domain specifically recognize a subunit of CD20. In some embodiments, the first target binding domain and second target binding domain specifically recognize a subunit of CD33. In some embodiments, the first target binding domain and second target binding domain specifically recognize a subunit of BCMA. In some embodiments, the first target binding domain and the second target binding domain specifically recognize a subunit of NKG2D. In some embodiments, the first target binding domain and second target binding domain specifically recognize a subunit of GPRC5D. In some embodiments, the first target binding domain and second target binding domain specifically recognize a subunit of NKG2D. In some embodiments, the first polypeptide further comprises a first intracellular domain of a first cytokine receptor, and/or the second polypeptide further comprises an intraceullar domain of a second cytokine receptor. In some embodiments, the modified immune cell further comprises and engineered receptor. In some embodiments, the engineered receptor comprises an extracellular domain specifically recognizing any of CD19, CLL1, BCMA, or GPC3. In some embodiments, the engineered receptor is a CAR, such as a CD19 CAR, a CLL1 CAR, a GPC3 CAR, or a BCMA CAR (e.g., a single BCMA CAR or a tandem BCMA CAR). In some embodiments, the engineered receptor is an engineered TCR. In some embodiments, the engineered receptor is a TAC receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling, and wherein the first target binding domain and the second binding domain each binds to the same target site on the target molecule. In some embodiments, the first polypeptide and the second polypeptide do not comprise an extracellular TGFβ-binding domain of a TGFβ (e.g., TGFβR1 and/or TGFβR2). In some embodiments, the first target binding domain and/or the second target binding domain is an antibody moiety or antigen-binding fragment thereof. In some embodiments, the first target binding domain and second target binding domain specifically recognize a subunit of CD20. In some embodiments, the first target binding domain and second target binding domain specifically recognize a subunit of CD33. In some embodiments, the first target binding domain and second target binding domain specifically recognize a subunit of BCMA. In some embodiments, the first target binding domain and the second target binding domain specifically recognize a subunit of NKG2D. In some embodiments, the first target binding domain and second target binding domain specifically recognize a subunit of GPRC5D. In some embodiments, the first target binding domain and second target binding domain specifically recognize a subunit of NKG2D. In some embodiments, the first polypeptide further comprises a first intracellular domain of a first cytokine receptor, and/or the second polypeptide further comprises an intraceullar domain of a second cytokine receptor. In some embodiments, the modified immune cell further comprises and engineered receptor. In some embodiments, the engineered receptor comprises an extracellular domain specifically recognizing any of CD19, CLL1, GPC3, and BCMA. In some embodiments, the engineered receptor is a CAR, such as a CD19 CAR, a CLL1 CAR, a GPC3 CAR, or a BCMA CAR (e.g., a single BCMA CAR or a tandem BCMA CAR). In some embodiments, the engineered receptor is an engineered TCR. In some embodiments, the engineered receptor is a TAC receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR4 transmembrane domain, and iii) a first TLR4 signaling domain; and b) a second polypeptide comprising: i) a second target binding domain, ii) a second TLR4 transmembrane domain, and iii) a second TLR4 signaling domain; wherein the first target binding domain and the second target binding domain each binds to a subunit of a multimeric target molecule, wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first target binding domain and the second target binding domain are each an extracellular NKG2D-binding domain of NKG2D. In some embodiments, the first target binding domain and/or the second target binding domain is an antibody moiety or antigen-binding fragment thereof. In some embodiments, the first target binding domain and/or second target binding domain is a scFv or sdAb. In some embodiments, the first polypeptide and the second polypeptide are the same. In some embodiments, the first target binding domain and the second target binding domain are each a scFv that specifically binds to CD20. In some embodiments, the first target binding domain and second target binding domain specifically recognize a subunit of CD33. In some embodiments, the first target binding domain and the second target binding domain are each a scFv that specifically binds to GPRC5D. In some embodiments, the first target binding domain and the second target binding domain are each a sdAb that specifically binds to BCMA. In some embodiments, the first polypeptide and the second polypeptide are different. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR2 transmembrane domain, and iii) a first TLR2 signaling domain; and b) a second polypeptide comprising i) a second target binding domain, ii) a second TLR1 transmembrane domain, and iii) a second TLR1 signaling domain; wherein the first target binding domain and the second target binding domain each binds to a subunit of a multimeric target molecule, wherein upon binding of the first target binding domain and second target binding domain to their corresponding target, the first TLR2 signaling domain and the second TLR1 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first target binding domain and/or the second target binding domain is an antibody moiety or antigen-binding fragment thereof. In some embodiments, the first target binding domain and/or second target binding domain is a scFv or sdAb. In some embodiments, the first target binding domain is an antibody moiety or antigen-binding fragment thereof. In some embodiments, the first target binding domain specifically recognizes the V subunit of CD33, and the second target binding domain specifically recognizes the C2 subunit of CD33. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first polypeptide comprises from the N-terminus to the C-terminus: a signal peptide, an anti-CD20 scFv, a TLR4 transmembrane domain, and a TLR4 signaling domain (e.g., the cytoplasmic portion of TLR4), and the second polypeptide comprises from the N-terminus to the C-terminus: a signal peptide, an anti-CD20 scFv, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first polypeptide comprises from the N-terminus to the C-terminus: a signal peptide, an anti-NKG2D extracellular domain (ECD), a TLR4 transmembrane domain, and a TLR4 signaling domain (e.g., the cytoplasmic portion of TLR4), and the second polypeptide comprises from the N-terminus to the C-terminus: a signal peptide, an anti-NKG2D ECD, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first polypeptide comprises from the N-terminus to the C-terminus: a signal peptide, an anti-GPRC5D scFv, a TLR4 transmembrane domain, and a TLR4 signaling domain (e.g., the cytoplasmic portion of TLR4), and the second polypeptide comprises from the N-terminus to the C-terminus: a signal peptide, an anti-GPRC5D scFv, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first polypeptide comprises from the N-terminus to the C-terminus: a signal peptide, an anti-BCMA sdAb, a TLR4 transmembrane domain, and a TLR4 signaling domain (e.g., the cytoplasmic portion of TLR4), and the second polypeptide comprises from the N-terminus to the C-terminus: a signal peptide, an anti-BCMA sdAb, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first polypeptide comprises from the N-terminus to the C-terminus: a signal peptide, an anti-CD33 sdAb, a TLR4 transmembrane domain, and a TLR4 signaling domain (e.g., the cytoplasmic portion of TLR4), and the second polypeptide comprises from the N-terminus to the C-terminus: a signal peptide, an anti-CD33 sdAb, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first polypeptide comprises from the N-terminus to the C-terminus: a signal peptide, an anti-CD33 V domain sdAb, a TLR2 transmembrane domain, and a TLR2 signaling domain (e.g., the cytoplasmic portion of TLR2), and the second polypeptide comprises from the N-terminus to the C-terminus: a signal peptide, an anti-CD33 C2 domain sdAb, a TLR1 transmembrane domain, and a TLR1 signaling domain (e.g., the cytoplasmic portion of TLR1), wherein upon binding of the first target binding domain and second target binding domain to their corresponding target, wherein the first TLR signaling domain and the second TLR signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first target binding domain and the second binding domain bind to the same target molecule, wherein the first target binding domain and the second target binding domain each binds to a different non-overlapping target site on a single target molecule, and wherein upon binding of the first target binding domain and second target binding domain to their corresponding target, the first TLR signaling domain and the second TLR signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first target binding domain and the second target binding domain are each an extracellular NKG2D-binding domain of NKG2D. In some embodiments, the first target binding domain and/or the second target binding domain is an antibody moiety or antigen-binding fragment thereof. In some embodiments, the first target binding domain and/or second target binding domain is a scFv or sdAb. In some embodiments, the subunits of the multimeric target molecule are the same. In some embodiments, the first target binding domain and second target binding domain specifically recognize a subunit of CD20. In some embodiments, the first target binding domain and the second target binding domain are each a scFv that specifically binds to GPRC5D. In some embodiments, the first target binding domain and the second target binding domain are each a sdAb that specifically binds to BCMA. In some embodiments, the first target binding domain and the second target binding domain are each a sdAb that specifically binds to CD33. In some embodiments, the first polypeptide further comprises a first intracellular domain of a first cytokine receptor, and/or the second polypeptide further comprises an intraceullar domain of a second cytokine receptor. In some embodiments, the modified immune cell further comprises and engineered receptor. In some embodiments, the engineered receptor comprises an extracellular domain specifically recognizing any of CD19, CLL1, GPC3, and BCMA. In some embodiments, the engineered receptor is a CAR, such as a CD19 CAR, a CLL1 CAR, a GPC3 CAR, or a BCMA CAR (e.g., a single BCMA CAR or a tandem BCMA CAR). In some embodiments, the engineered receptor is an engineered TCR. In some embodiments, the engineered receptor is a TAC receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR4 transmembrane domain, and iii) a first TLR4 signaling domain; and b) a second polypeptide comprising i) a second target binding domain, ii) a second TLR4 transmembrane domain, and iii) a second TLR4 signaling domain; wherein the first target binding domain and the second binding domain bind to the same target molecule, wherein the first target binding domain and the second target binding domain each binds to a different non-overlapping target site on a single target molecule, wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first target binding domain and the second target binding domain are each an extracellular NKG2D-binding domain of NKG2D. In some embodiments, the first target binding domain and/or the second target binding domain is an antibody moiety or antigen-binding fragment thereof. In some embodiments, the first target binding domain and/or second target binding domain is a scFv or sdAb. In some embodiments, the first polypeptide and the second polypeptide are the same. In some embodiments, the first target binding domain and the second target binding domain are each a scFv that specifically binds to CD20. In some embodiments, the first target binding domain and the second target binding domain are each a scFv that specifically binds to GPRC5D. In some embodiments, the first target binding domain and the second target binding domain are each a sdAb that specifically binds to BCMA. In some embodiments, the first target binding domain and the second target binding domain are each a sdAb that specifically binds to CD33. In some embodiments, the first polypeptide and the second polypeptide are different. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR2 transmembrane domain, and iii) a first TLR2 signaling domain; and b) a second polypeptide comprising i) a second target binding domain, ii) a second TLR1 transmembrane domain, and iii) a second TLR1 signaling domain; wherein the first target binding domain and the second binding domain bind to the same target molecule, wherein the first target binding domain and the second target binding domain each binds to a different non-overlapping target site on a single target molecule, wherein upon binding of the first target binding domain and second target binding domain to their corresponding target, the first TLR2 signaling domain and the second TLR1 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first target binding domain and/or the second target binding domain is an antibody moiety or antigen-binding fragment thereof. In some embodiments, the first target binding domain and/or second target binding domain is a scFv or sdAb. In some embodiments, the first target binding domain is an antibody moiety or antigen-binding fragment thereof. In some embodiments, the first target binding domain specifically recognizes the V subunit of CD33, and the second target binding domain specifically recognizes the C2 subunit of CD33. In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first target binding domain and the second binding domain bind to the same target molecule, wherein the first target binding domain and the second target binding domain each binds to a different non-overlapping target site on a single target molecule, wherein the first polypeptide comprises from the N-terminus to the C-terminus: a signal peptide, an anti-CD20 scFv, a TLR4 transmembrane domain, and a TLR4 signaling domain (e.g., the cytoplasmic portion of TLR4), and the second polypeptide comprises from the N-terminus to the C-terminus: a signal peptide, an anti-CD20 scFv, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first target binding domain and the second binding domain bind to the same target molecule, wherein the first target binding domain and the second target binding domain each binds to a different non-overlapping target site on a single target molecule, wherein the first polypeptide comprises from the N-terminus to the C-terminus: a signal peptide, an anti-NKG2D ECD, a TLR4 transmembrane domain, and a TLR4 signaling domain (e.g., the cytoplasmic portion of TLR4), and the second polypeptide comprises from the N-terminus to the C-terminus: a signal peptide, an anti-NKG2D ECD, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first target binding domain and the second binding domain bind to the same target molecule, wherein the first target binding domain and the second target binding domain each binds to a different non-overlapping target site on a single target molecule, wherein the first polypeptide comprises from the N-terminus to the C-terminus: a signal peptide, an anti-BCMA sdAb, a TLR4 transmembrane domain, and a TLR4 signaling domain (e.g., the cytoplasmic portion of TLR4), and the second polypeptide comprises from the N-terminus to the C-terminus: a signal peptide, an anti-BCMA sdAb, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first target binding domain and the second binding domain bind to the same target molecule, wherein the first target binding domain and the second target binding domain each binds to a different non-overlapping target site on a single target molecule, wherein the first polypeptide comprises from the N-terminus to the C-terminus: a signal peptide, an anti-GPRC5D scFv, a TLR4 transmembrane domain, and a TLR4 signaling domain (e.g., the cytoplasmic portion of TLR4), and the second polypeptide comprises from the N-terminus to the C-terminus: a signal peptide, an anti-GPRC5D scFv, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first target binding domain and the second binding domain bind to the same target molecule, wherein the first target binding domain and the second target binding domain each binds to a different non-overlapping target site on a single target molecule, wherein the first polypeptide comprises from the N-terminus to the C-terminus: a signal peptide, an anti-CD33 sdAb, a TLR4 transmembrane domain, and a TLR4 signaling domain (e.g., the cytoplasmic portion of TLR4), and the second polypeptide comprises from the N-terminus to the C-terminus: a signal peptide, an anti-CD33 sdAb, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first target binding domain and the second binding domain bind to the same target molecule, wherein the first target binding domain and the second target binding domain each binds to a different non-overlapping target site on a single target molecule, wherein the first polypeptide comprises from the N-terminus to the C-terminus: a signal peptide, an anti-CD33 V domain sdAb, a TLR2 transmembrane domain, and a TLR2 signaling domain (e.g., the cytoplasmic portion of TLR2), and the second polypeptide comprises from the N-terminus to the C-terminus: a signal peptide, an anti-CD33 C2 domain sdAb, a TLR1 transmembrane domain, and a TLR1 signaling domain (e.g., the cytoplasmic portion of TLR1), and wherein upon binding of the first target binding domain and second target binding domain to their corresponding target, the first TLR signaling domain and the second TLR signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a a) a first polypeptide comprising: i) a first target binding domain, ii) a first TLR transmembrane domain, and iii) a first TLR signaling domain; b) a second polypeptide comprising: i) a second target binding domain, ii) a second TLR transmembrane domain, and iii) a second TLR signaling domain; and c) an engineered receptor, wherein the engineered receptor comprises an extracellular domain specifically recognizing the same target molecule as the first polypeptide and/or the second polypeptide, and wherein upon binding of the first target binding domain and second target binding domain to their corresponding target, the first TLR signaling domain and the second TLR signaling domain associate with each other to form a TLR signaling moiety capable of inducing TLR signaling. In some embodiments, the engineered receptor comprises an extracellular domain specifically recognizing the same target molecule as the first polypeptide and the second polypeptide. In some embodiments, the engineered receptor comprises an extracellular domain specifically recognizing the same target molecule as the first polypeptide, which is different from the target molecule of the second polypeptide. In some embodiments, the engineered receptor comprises an extracellular domain specifically recognizing the same target molecule as the second polypeptide, which is different from the target molecule of the first polypeptide. In some embodiments, the first polypeptide and the second polypeptide do not comprise an extracellular TGFβ-binding domain of a TGFβ (e.g., TGFβR1 and/or TGFβR2). In some embodiments, the first target binding domain and/or the second target binding domain is an antibody moiety or antigen-binding fragment thereof. In some embodiments, the first target binding domain and the second target binding domain each binds to a subunit of a multimeric target molecule. In some embodiments, the subunits of the multimeric target molecule are different. In some embodiments, the first target binding domain specifically recognizes the V subunit of CD33, and the second target binding domain specifically recognizes the C2 subunit of CD33. In some embodiments, the subunits of the multimeric target molecule are the same. In some embodiments, the first target binding domain and second target binding domain specifically recognize a subunit of CD20. In some embodiments, the engineered receptor comprises an extracellular domain specifically recognizing a non-overlapping target site on the same target molecule as the first polypeptide and/or the second polypeptide. In some embodiments, the first polypeptide further comprises a first intracellular domain of a first cytokine receptor, and/or the second polypeptide further comprises an intraceullar domain of a second cytokine receptor. In some embodiments, the engineered receptor comprises an extracellular domain specifically recognizing any of CD19, CLL1, GPC3 and BCMA. In some embodiments, the engineered receptor is a CAR, such as a CD19 CAR, a CLL1 CAR, a GPC3 CAR or a BCMA CAR. In some embodiments, the engineered receptor is an engineered TCR. In some embodiments, the engineered receptor is a TAC receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR4 transmembrane domain, and iii) a first TLR4 signaling domain; b) a second polypeptide comprising: i) a second target binding domain, ii) a second TLR4 transmembrane domain, and iii) a second TLR4 signaling domain; and c) an engineered receptor, wherein the engineered receptor comprises an extracellular domain specifically recognizing the same target molecule as the first polypeptide and/or the second polypeptide, wherein the engineered receptor comprises an extracellular domain specifically recognizing the same target molecule as the first polypeptide and/or the second polypeptide, and wherein upon binding of the first target binding domain and second target binding domain to their corresponding target, the first TLR4 signaling domain and the second TLR4 signaling domain associate with each other to form a TLR signaling moiety capable of inducing TLR signaling In some embodiments, the first polypeptide and the second polypeptide do not comprise an extracellular TGFβ-binding domain of a TGFβ (e.g., TGFβR1 and/or TGFβR2). In some embodiments, the first target binding domain and the second target binding domain are each an extracellular NKG2D-binding domain of NKG2D. In some embodiments, the first target binding domain and/or the second target binding domain is an antibody moiety or antigen-binding fragment thereof. In some embodiments, the first target binding domain and/or second target binding domain is a scFv or sdAb. In some embodiments, the first polypeptide and the second polypeptide are the same. In some embodiments, the first target binding domain and the second target binding domain are each a scFv that specifically binds to CD20. In some embodiments, the first target binding domain and the second target binding domain are each a scFv that specifically binds to GPRC5D. In some embodiments, the first target binding domain and the second target binding domain are each a sdAb that specifically binds to BCMA. In some embodiments, the first target binding domain and the second target binding domain are each a sdAb that specifically binds to CD33. In some embodiments, the first polypeptide and the second polypeptide are different. In some embodiments, the first polypeptide further comprises a first intracellular domain of a first cytokine receptor, and/or the second polypeptide further comprises an intraceullar domain of a second cytokine receptor. In some embodiments, the engineered receptor is a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell comprising a) a first polypeptide comprising: i) a first target binding domain, ii) a first TLR2 transmembrane domain, and iii) a first TLR2 signaling domain; b) a second polypeptide comprising: i) a second target binding domain, ii) a second TLR1 transmembrane domain, and iii) a second TLR1 signaling domain; and c) an engineered receptor, wherein the engineered receptor comprises an extracellular domain specifically recognizing the same target molecule as the first polypeptide and/or the second polypeptide, wherein the engineered receptor comprises an extracellular domain specifically recognizing the same target molecule as the first polypeptide and/or the second polypeptide, and wherein upon binding of the first target binding domain and second target binding domain to their corresponding target, the first TLR2 signaling domain and the second TLR1 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first target binding domain and/or the second target binding domain is an antibody moiety or antigen-binding fragment thereof. In some embodiments, the first target binding domain and/or second target binding domain is a scFv or sdAb. In some embodiments, the first target binding domain is an antibody moiety or antigen-binding fragment thereof. In some embodiments, the first target binding domain specifically recognizes the V subunit of CD33, and the second target binding domain specifically recognizes the C2 subunit of CD33. In some embodiments, the first polypeptide further comprises a first intracellular domain of a first cytokine receptor, and/or the second polypeptide further comprises an intraceullar domain of a second cytokine receptor. In some embodiments, the engineered receptor is a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; b) a second polypeptide comprising: i) a second target binding domain, ii) a second TLR transmembrane domain, and iii) a second TLR signaling domain; and c) an engineered receptor, wherein the engineered receptor comprises an extracellular domain specifically recognizing the same target molecule as the first polypeptide and/or the second polypeptide, wherein the engineered receptor comprises an extracellular domain specifically recognizing the same target molecule as the first polypeptide and/or the second polypeptide, wherein the first polypeptide comprises from the N-terminus to the C-terminus: a signal peptide, an anti-CD20 scFv, a TLR4 transmembrane domain, and a TLR4 signaling domain (e.g., the cytoplasmic portion of TLR4), and the second polypeptide comprises from the N-terminus to the C-terminus: a signal peptide, an anti-CD20 scFv, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the engineered receptor is a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; b) a second polypeptide comprising: i) a second target binding domain, ii) a second TLR transmembrane domain, and iii) a second TLR signaling domain; and c) an engineered receptor, wherein the engineered receptor comprises an extracellular domain specifically recognizing the same target molecule as the first polypeptide and/or the second polypeptide, wherein the engineered receptor comprises an extracellular domain specifically recognizing the same target molecule as the first polypeptide and/or the second polypeptide, wherein the first polypeptide comprises from the N-terminus to the C-terminus: a signal peptide, an anti-NKG2D ECD, a TLR4 transmembrane domain, and a TLR4 signaling domain (e.g., the cytoplasmic portion of TLR4), and the second polypeptide comprises from the N-terminus to the C-terminus: a signal peptide, an anti-NKG2D ECD, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the engineered receptor is a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; b) a second polypeptide comprising: i) a second target binding domain, ii) a second TLR transmembrane domain, and iii) a second TLR signaling domain; and c) an engineered receptor, wherein the engineered receptor comprises an extracellular domain specifically recognizing the same target molecule as the first polypeptide and/or the second polypeptide, wherein the engineered receptor comprises an extracellular domain specifically recognizing the same target molecule as the first polypeptide and/or the second polypeptide, wherein the first polypeptide comprises from the N-terminus to the C-terminus: a signal peptide, an anti-GPRC5D scFv, a TLR4 transmembrane domain, and a TLR4 signaling domain (e.g., the cytoplasmic portion of TLR4), and the second polypeptide comprises from the N-terminus to the C-terminus: a signal peptide, an anti-GPRC5D scFv, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the engineered receptor is a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; b) a second polypeptide comprising: i) a second target binding domain, ii) a second TLR transmembrane domain, and iii) a second TLR signaling domain; and c) an engineered receptor, wherein the engineered receptor comprises an extracellular domain specifically recognizing the same target molecule as the first polypeptide and/or the second polypeptide, wherein the engineered receptor comprises an extracellular domain specifically recognizing the same target molecule as the first polypeptide and/or the second polypeptide, wherein the first polypeptide comprises from the N-terminus to the C-terminus: a signal peptide, an anti-BCMA sdAb, a TLR4 transmembrane domain, and a TLR4 signaling domain (e.g., the cytoplasmic portion of TLR4), and the second polypeptide comprises from the N-terminus to the C-terminus: a signal peptide, an anti-BCMA sdAb, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the engineered receptor is a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; b) a second polypeptide comprising: i) a second target binding domain, ii) a second TLR transmembrane domain, and iii) a second TLR signaling domain; and c) an engineered receptor, wherein the engineered receptor comprises an extracellular domain specifically recognizing the same target molecule as the first polypeptide and/or the second polypeptide, wherein the engineered receptor comprises an extracellular domain specifically recognizing the same target molecule as the first polypeptide and/or the second polypeptide, wherein the first polypeptide comprises from the N-terminus to the C-terminus: a signal peptide, an anti-CD33 sdAb, a TLR4 transmembrane domain, and a TLR4 signaling domain (e.g., the cytoplasmic portion of TLR4), and the second polypeptide comprises from the N-terminus to the C-terminus: a signal peptide, an anti-CD33 sdAb, a TLR4 transmembrane domain, and a TLR4 signaling domain (e.g., the cytoplasmic portion of TLR4), and wherein upon binding of the first target binding domain and second target binding domain to their corresponding target, the first TLR4 signaling domain and the second TLR4 signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the engineered receptor is a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a modified immune cell (e.g., T cell) comprising a) a first polypeptide comprising: i) a first target binding domain, ii) a first TLR transmembrane domain, and iii) a first TLR signaling domain; b) a second polypeptide comprising: i) a second target binding domain, ii) a second TLR transmembrane domain, and iii) a second TLR signaling domain; and c) an engineered receptor, wherein the engineered receptor comprises an extracellular domain specifically recognizing the same target molecule as the first polypeptide and/or the second polypeptide, wherein the engineered receptor comprises an extracellular domain specifically recognizing the same target molecule as the first polypeptide and/or the second polypeptide, wherein the first polypeptide comprises from the N-terminus to the C-terminus: a signal peptide, an anti-CD33 V domain sdAb, a TLR2 transmembrane domain, and a TLR2 signaling domain (e.g., the cytoplasmic portion of TLR2), and the second polypeptide comprises from the N-terminus to the C-terminus: a signal peptide, an anti-CD33 C2 domain sdAb, a TLR1 transmembrane domain, and a TLR1 signaling domain (e.g., the cytoplasmic portion of TLR1), and wherein upon binding of the first target binding domain and second target binding domain to their corresponding target, the first TLR signaling domain and the second TLR signaling domain associate with each other to form a TLR signaling moiety capable of inducing TLR signaling. In some embodiments, the first polypeptide and the second polypeptide do not comprise an extracellular TGFβ-binding domain of a TGFβ (e.g., TGFβR1 and/or TGFβR2). In some embodiments, the first polypeptide further comprises a first intracellular domain of a first cytokine receptor, and/or the second polypeptide further comprises an intraceullar domain of a second cytokine receptor. In some embodiments, the engineered receptor is a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a CAR-expressing immune cell (e.g., CAR-T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the first target binding domain and the second target binding domain each binds to a subunit of a multimeric target molecule. In some embodiments, the subunits of the multimeric target molecule are different. In some embodiments, the subunits of the multimeric target molecule are the same. In some embodiments, the first target binding domain and the second binding domain bind to the same target molecule. In some embodiments, the first target binding domain and the second binding domain each binds to the same target site on the target molecule. In some embodiments, the first target binding domain and the second target binding domain are the same. In some embodiments, wherein the first target binding domain and the second binding domain bind to the same target molecule, the first target binding domain and the second target binding domain each binds to a different non-overlapping target sites on a single target molecule. In some embodiments, the CAR comprises an extracellular domain specifically recognizing the same target molecule as the first polypeptide and/or the second polypeptide. In some embodiments, the CAR comprises an extracellular domain specifically recognizing a non-overlapping target site on the same target molecule as the first polypeptide and/or the second polypeptide. In some embodiments, the CAR comprises an extracellular domain specifically recognizing any of CD19, CLL1, GPC3, and BCMA (e.g., a single BCMA CAR or a tandem BCMA CAR). In some embodiments, the first polypeptide further comprises a first intracellular domain of a first cytokine receptor, and/or the second polypeptide further comprises an intraceullar domain of a second cytokine receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a TCR-expressing immune cell (e.g., TCR-T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the first target binding domain and the second target binding domain each binds to a subunit of a multimeric target molecule. In some embodiments, the subunits of the multimeric target molecule are different. In some embodiments, the subunits of the multimeric target molecule are the same. In some embodiments, the first target binding domain and the second binding domain bind to the same target molecule. In some embodiments, the first target binding domain and the second binding domain each binds to the same target site on the target molecule. In some embodiments, the first target binding domain and the second target binding domain are the same. In some embodiments, wherein the first target binding domain and the second binding domain bind to the same target molecule, the first target binding domain and the second target binding domain each binds to a different non-overlapping target sites on a single target molecule. In some embodiments, the TCR comprises an extracellular domain specifically recognizing the same target molecule as the first polypeptide and/or the second polypeptide. In some embodiments, the TCR comprises an extracellular domain specifically recognizing a non-overlapping target site on the same target molecule as the first polypeptide and/or the second polypeptide. In some embodiments, the TCR comprises an extracellular domain specifically recognizing any of CD19, CLL1, GPC3, and BCMA (e.g., a single BCMA TCR or a tandem BCMA TCR). In some embodiments, the first polypeptide further comprises a first intracellular domain of a first cytokine receptor, and/or the second polypeptide further comprises an intraceullar domain of a second cytokine receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a TAC-expressing immune cell (e.g., TAC-T cell) comprising a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the first target binding domain and the second target binding domain each binds to a subunit of a multimeric target molecule. In some embodiments, the subunits of the multimeric target molecule are different. In some embodiments, the subunits of the multimeric target molecule are the same. In some embodiments, the first target binding domain and the second binding domain bind to the same target molecule. In some embodiments, the first target binding domain and the second binding domain each binds to the same target site on the target molecule. In some embodiments, the first target binding domain and the second target binding domain are the same. In some embodiments, wherein the first target binding domain and the second binding domain bind to the same target molecule, the first target binding domain and the second target binding domain each binds to a different non-overlapping target sites on a single target molecule. In some embodiments, the TAC comprises an extracellular domain specifically recognizing the same target molecule as the first polypeptide and/or the second polypeptide. In some embodiments, the TAC comprises an extracellular domain specifically recognizing a non-overlapping target site on the same target molecule as the first polypeptide and/or the second polypeptide. In some embodiments, the TAC comprises an extracellular domain specifically recognizing any of CD19, CLL1, GPC3, and BCMA (e.g., a single BCMA TAC or a tandem BCMA TAC). In some embodiments, the first polypeptide further comprises a first intracellular domain of a first cytokine receptor, and/or the second polypeptide further comprises an intraceullar domain of a second cytokine receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a CAR-expressing immune cell (e.g., CAR-T cell) comprising a first polypeptide comprising: i) a first target binding domain (e.g., an antibody moiety or antigen binding fragment thereof), ii) a first TLR4 transmembrane domain, and iii) a first TLR4 signaling domain, and b) a second polypeptide comprising: i) a second target binding domain (e.g., an antibody moiety or antigen binding fragment thereof), ii) a second TLR4 transmembrane domain, and iii) a second TLR4 signaling domain, wherein upon binding of the target binding domain and second target binding domain to their corresponding target, the first TLR4 signaling domain and the second TLR4 signaling domain associate with each other to form a TLR4 signaling moiety capable of inducing TLR4 signaling. In some embodiments, the first polypeptide further comprises a signal peptide (e.g., leader sequence). In some embodiments, the first polypeptide and the second polypeptide are the same. In some embodiments, each of the first polypeptide and the second polypeptide comprises from the N-terminus to the C-terminus: a signal peptide, an anti-CD20 scFv, a TLR4 transmembrane domain, and a TLR4 signaling domain (e.g., the cytoplasmic portion of TLR4). In some embodiments, each of the first polypeptide and the second polypeptide comprises from the N-terminus to the C-terminus: a signal peptide, an anti-BCMA sdAb, a TLR4 transmembrane domain, and a TLR4 signaling domain (e.g., the cytoplasmic portion of TLR4). In some embodiments, each of the first polypeptide and the second polypeptide comprises from the N-terminus to the C-terminus: a signal peptide, an anti-GPRC5D scFv, a TLR4 transmembrane domain, and a TLR4 signaling domain (e.g., the cytoplasmic portion of TLR4). In some embodiments, each of the first polypeptide and the second polypeptide comprises from the N-terminus to the C-terminus: a signal peptide, an anti-NKG2D ECD, a TLR4 transmembrane domain, and a TLR4 signaling domain (e.g., the cytoplasmic portion of TLR4). In some embodiments, each of the first polypeptide and the second polypeptide comprises from the N-terminus to the C-terminus: a signal peptide, an anti-CD33 sdAb, a TLR4 transmembrane domain, and a TLR4 signaling domain (e.g., the cytoplasmic portion of TLR4). In some embodiments, the modified immune cell expresses an anti-CD19 CAR. In some embodiments, the anti-CD19 CAR comprises from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-CD19 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 2. In some embodiments, the anti-CD19 CAR comprises SEQ ID NO: 2. In some embodiments, the modified immune cell expresses an anti-CLL1 CAR. In some embodiments, the anti-CLL1 CAR comprises from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain. In some embodiments, the anti-CLL1 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 4. In some embodiments, the anti-CLL1 CAR comprises SEQ ID NO: 4. In some embodiments, the modified immune cell expresses an anti-BCMA CAR (e.g., a single anti-BCMA CAR or a tandem anti-BCMA CAR). In some embodiments, the anti-BCMA CAR comprises from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-BCMA CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 67. In some embodiments, the anti-BCMA CAR comprises SEQ ID NO: 67. In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the anti-BCMA CAR comprises from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-BCMA CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 66. In some embodiments, the anti-BCMA CAR comprises SEQ ID NO: 66. In some embodiments, the modified immune cell expresses an anti-GPC3 CAR. In some embodiments, the anti-GPC3 CAR comprises from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-GPC3 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 56. In some embodiments, the anti-GPC3 CAR comprises SEQ ID NO: 56. In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the modified immune cell comprises a CAR system (e.g., a CAR fusion construct), wherein the CAR fusion construct comprises an anti-CD19 CAR and anti-CD20 TLR polypeptides. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD20 scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1. In some embodiments, the CAR fusion construct comprises SEQ ID NO: 1. In some embodiments, the modified immune cell comprises a CAR system (e.g., a CAR fusion construct), wherein the CAR fusion construct comprises an anti-CLL1 CAR and anti-CD33 TLR polypeptides. In some embodiments, the CAR fusion construct comprises, from the N-terminus to the C-terminus: a signal peptide, an anti-CLL1 sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD33 sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 71. In some embodiments, the CAR fusion construct comprises SEQ ID NO: 71. In some embodiments, the modified immune cell comprises a CAR system (e.g., a CAR fusion construct), wherein the CAR fusion construct comprises an anti-CD19 CAR and anti-NKG2D TLR polypeptides. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 59. In some embodiments the CAR fusion construct comprises SEQ ID NO: 59. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 60. In some embodiments the CAR fusion construct comprises SEQ ID NO: 60. In some embodiments, the modified immune cell comprises a CAR system (e.g., a CAR fusion construct), wherein the CAR fusion construct comprises an anti-GPC3 CAR and anti-NKG2D TLR polypeptides. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 57. In some embodiments the CAR fusion construct comprises SEQ ID NO: 57. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 58. In some embodiments the CAR fusion construct comprises SEQ ID NO: 58. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-BCMA TLR polypeptides. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 62. In some embodiments the CAR fusion construct comprises SEQ ID NO: 62. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 63. In some embodiments the CAR fusion construct comprises SEQ ID NO: 63. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 64. In some embodiments the CAR fusion construct comprises SEQ ID NO: 64. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a tandem anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 61. In some embodiments the CAR fusion construct comprises SEQ ID NO: 61. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-GPRC5D TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 65. In some embodiments the CAR fusion construct comprises SEQ ID NO: 65. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 72. In some embodiments the CAR fusion construct comprises SEQ ID NO: 72. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 73. In some embodiments the CAR fusion construct comprises SEQ ID NO: 73. In some embodiments, the first polypeptide further comprises a first intracellular domain of a first cytokine receptor, and/or the second polypeptide further comprises an intraceullar domain of a second cytokine receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a CAR-expressing immune cell (e.g., CAR-T cell) comprising a) a first polypeptide comprising: i) a first target binding domain (e.g., an antibody moiety or antigen binding fragment thereof), ii) a TLR2 transmembrane domain, and iii) a first TLR2 signaling domain; and b) a second polypeptide comprising: i) a second target binding domain (e.g., an antibody moiety or antigen binding fragment thereof), ii) a TLR1 transmembrane domain, and iii) a TLR1 signaling domain, wherein upon binding of the first target binding domain and second target binding domain to their corresponding target, the TLR1 signaling domain and the TLR2 signaling domain associate with each other to form a TLR signaling moiety capable of inducing TLR signaling. In some embodiments, the first polypeptide and/or the second polypeptide further comprises a signal peptide (e.g., leader sequence). In some embodiments, the first polypeptide comprises from the N-terminus to the C-terminus: a signal peptide, an anti-CD33 V domain sdAb, a TLR2 transmembrane domain, and a TLR2 signaling domain (e.g., the cytoplasmic portion of TLR2), and the second polypeptides comprises from the N-terminus to the C-terminus: a signal peptide, an anti-CD33 C2 domain sdAb, a TLR1 transmembrane domain, and a TLR1 signaling domain (e.g., the cytoplasmic portion of TLR1). In some embodiments, the modified immune cell expresses an anti-CLL1 CAR. In some embodiments, the modified immune cell expresses an anti-CLL1/CD33 dual CAR. In some embodiments, the anti-CLL1/CD33 dual CAR comprises from the N-terminus to the C-terminus: a signal peptide, an anti-CLL1 sdAb, an anti-CD33 V domain sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-CLL1/CD33 dual CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 43. In some embodiments the anti-CLL1/CD33 dual CAR comprises SEQ ID NO: 43. In some embodiments, the first polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 41. In some embodiments the first polypeptide comprises SEQ ID NO: 41. In some embodiments, the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 42. In some embodiments the second polypeptide comprises SEQ ID NO: 42. In some embodiments, the modified immune cell comprises a CAR system (e.g., a CAR fusion construct), wherein the CAR fusion construct comprises an anti-CLL1 CAR and anti-CD33 (e.g., anti-CD33 V domain and/or anti-CD33 C2 domain) TLR polypeptides. In some embodiments, the CAR fusion construct comprises, from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain, a P2A cleavage site, a signal peptide, an anti-CD33 V domain sdAb, a TLR2 transmembrane domain, a TLR2 signaling domain (e.g., the cytoplasmic portion of TLR2), a P2A cleavage site, a signal peptide, an anti-CD33 C2 domain sdAb, a TLR1 transmembrane domain, and a TLR1 signaling domain (e.g., the cytoplasmic portion of TLR). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 3. In some embodiments the CAR fusion construct comprises SEQ ID NO: 3. In some embodiments, the first polypeptide further comprises a first intracellular domain of a first cytokine receptor, and/or the second polypeptide further comprises an intraceullar domain of a second cytokine receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

### Immune Cells

The modified immune cell can be derived from a variety of cell types and cell sources. Cells from any mammalian species, including, but not limited to, mice, rats, guinea pigs, rabbits, dogs, monkeys, and humans, are contemplated herein. In some embodiments, the modified immune cell is a human cell. In some embodiments, the modified immune cell is allogenic (i.e., from the same species, but different donor) as the recipient individual. In some embodiments, the modified immune cell is autologous (i.e., the donor and the recipient are the same). In some embodiments, the modified immune cell is syngeneic (i.e., the donor and the recipients are different individuals, but are identical twins).

In some embodiments, the modified immune cell is derived from a primary cell. In some embodiments, the modified immune cell is a primary cell isolated from an individual. In some embodiments, the modified immune cell is propagated (such as proliferated and/or differentiated) from a primary cell isolated from an individual. In some embodiments, the primary cell is of the hematopoietic lineage. In some embodiments, the primary cell is obtained from the thymus. In some embodiments, the primary cell is obtained from the lymph or lymph nodes (such as tumor draining lymph nodes). In some embodiments, the primary cell is obtained from the spleen. In some embodiments, the primary cell is obtained from the bone marrow. In some embodiments, the primary cell is obtained from the blood, such as the peripheral blood. In some embodiments, the primary cell is a Peripheral Blood Mononuclear Cell (PBMC). In some embodiments, the primary cell is derived from the blood plasma. In some embodiments, the primary cell is derived from a tumor. In some embodiments, the primary cell is obtained from the mucosal immune system. In some embodiments, the primary cell is obtained from a biopsy sample.

In some embodiments, the modified immune cell is derived from a cell line. In some embodiments, the modified immune cell is obtained from a commercial cell line. In some embodiments, the modified immune cell is a cell line established from a primary cell isolated from an individual. In some embodiments, the modified immune cell is propagated (such as proliferated and/or differentiated) from a cell line. In some embodiments, the cell line is mortal. In some embodiments, the cell line is immortalized. In some embodiments, the cell line is a tumor cell line, such as a leukemia or lymphoma cell line. In some embodiments, the cell line is a cell line derived from the PBMC. In some embodiments, the cell line is a stem cell line. In some embodiments, the cell line is selected from the group consisting of HEK293-6E cells, NK-92 cells, and Jurkat cells.

Exemplary immune cells useful for the present application include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lyphokine-activated killer (LAK) cells), B cells, Natural Killer (NK) cells, monocytes, macrophages, neutrophils, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of modified immune cells, the modified immune cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enhanced in the immune cell. In some embodiments, the modified immune cell is a lymphocyte. In some embodiments, the modified immune cell is not a lymphocyte. In some embodiments, the modified immune cell is suitable for adoptive immunotherapy. In some embodiments, the modified immune cell is a PBMC. In some embodiments, the modified immune cell is an immune cell derived from the PBMC. In some embodiments, the modified immune cell is a T cell. In some embodiments, the modified immune cell is a CD4⁺ T cell. In some embodiments, the modified immune cell is a CD8⁺ T cell. In some embodiments, the modified immune cell is a B cell. In some embodiments, the modified immune cell is an NK cell.

In some embodiments, the modified immune cell is derived from a stem cell. In some embodiments, the stem cell is a totipotent stem cell. In some embodiments, the stem cell is a pluripotent stem cell. In some embodiments, the stem cell is a unipotent stem cell. In some embodiments, the stem cell is a progenitor cell. In some embodiments, the stem cell is an embryonic stem cell. In some embodiments, the stem cell is hematopoietic stem cell. In some embodiments, the stem cell is a mesenchymal stem cell. In some embodiments, the stem cell is an induced pluripotent stem cell (iPSC).

The modified immune cell may comprise any number (such as any of 1, 2, 3, 4, 5, 10, 50, 100, 1000, or more) of the heterologous nucleic acid sequence (including first and second nucleic acid sequences). In some embodiments, the modified immune cell comprises a single copy of the first and/or second heterologous nucleic acid sequence. In some embodiments, the modified immune cell comprises a plurality of copies of the first and/or second heterologous nucleic acid sequence. In some embodiments, the modified immune cell further comprises at least one additional heterologous nucleic acid sequence, for example, a heterologous nucleic acid sequence encoding an immunomodulatory agent, such as cytokine, chemokine, and/or an immune checkpoint inhibitor.

Nucleic acid(s) comprising the heterologous nucleic acid sequence(s) described herein may be transiently or stably incorporated in the modified immune cell. In some embodiments, the nucleic acid(s) is transiently expressed in the modified immune cell. For example, the nucleic acid(s) may be present in the nucleus of the modified immune cell in an extrachromosomal array. The nucleic acid(s) may be introduced into the modified immune cell using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection.

In some embodiments, the heterologous nucleic acid sequence(s) is present in the genome of the modified immune cell. For example, nucleic acid(s) comprising the heterologous nucleic acid sequence(s) may be integrated into the genome of the modified immune cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. In some embodiments, the heterologous nucleic acid sequence(s) is integrated in a specifically designed locus of the genome of the modified immune cell. In some embodiments, the heterologous nucleic acid sequence(s) is integrated in an integration hotspot of the genome of the modified immune cell. In some embodiments, the heterologous nucleic acid (sequence) is integrated in a random locus of the genome of the modified immune cell. In the cases that multiple copies of the heterologous nucleic acid sequence(s) are present in a single modified immune cell, the heterologous nucleic acid sequences may be integrated in a plurality of loci of the genome of the modified immune cell.

### TLR Polypeptides

The modified immune cells described herein express toll-like receptor (TLR) polypeptides (e.g., a first TLR polypeptide and a second TLR polypeptide; also referred herein as a first polypeptide and a second polypeptide, respectively). In some embodiments, the modified immune cells express at least one TLR polypeptide. In some embodiments, the modified immune cells express two TLR polypeptides. The present application also provides TLR polypeptides and compositions thereof. In some embodiments, the TLR polypeptides provided herein provide strong co-stimulatory anti-tumor effects by inducing TLR signaling after antigen stimulation.

The TLR polypeptides are derived from TLR molecules. TLRs are type I transmembrane glycoproteins characterized by the presence of an extracellular domain containing leucine rich repeats (LRRs), which is responsible for mediating ligand recognition. The extracellular domain is followed by a single transmembrane helix and an intracellular Toll-like/interleukin-1 (IL-1) receptor (TIR) domain. In some embodiments, the TIR domain is responsible for downstream signaling (e.g., inducing TLR signaling). The domains and function of TLRs are well known in the art. See, e.g., Jin et al., 2008 29(2):182-91;

In some embodiments, the TLR molecules is selected from the group consisting of TLR1, TLR2, TLR3, TLR4, TLR5, TLR6, TLR7, TLR8, TLR9, and TLR10. In some embodiments, the TLR polypeptide is derived from TLR1. In some embodiments, the TLR polypeptide is derived from TLR2. In some embodiments, the TLR polypeptide is derived from TLR4. In some embodiments, the TLR polypeptide is derived from TLR6.

In some embodiments, the TLR polypeptides dimerize upon binding of the first target binding domain and second target binding domain to their corresponding target, which result in association of the first TLR signaling domain and the second TLR signaling domain to form a TLR signaling moiety capable of inducing TLR signaling. In some embodiments, the TLR polypeptides form a homodimer (e.g., dimerization of two of the same TLR molecules). Homodimers can form with TLR polypeptides derived from TLR3, TLR4, TLR5, or TLR9. In some embodiments, each of the first and the second TLR polypeptides comprises a TLR3 transmembrane domain and a TLR3 signaling domain. In some embodiments, each of the first and the second TLR polypeptides comprises a TLR4 transmembrane domain and a TLR4 signaling domain. In some embodiments, each of the first and the second TLR polypeptides comprises a TLR5 transmembrane domain and a TLR5 signaling domain. In some embodiments, each of the first and the second TLR polypeptides comprises a TLR9 transmembrane domain and a TLR9 signaling domain.

In some embodiments, the TLR polypeptides form a heterodimer (e.g., dimerization of two different TLR polypeptides). Heterodimers can form with TLR polypeptides derived from TLR1/TLR2, TLR1/TLR4, TLR2/TLR6, TLR2/TLR10, or TLR4/TLR5. In some embodiments, the first TLR polypeptide comprises a TLR1 transmembrane domain and a TLR1 signaling domain, and the second TLR polypeptide comprises a TLR2 transmembrane domain and a TLR2 signaling domain; or the first TLR polypeptide comprises a TLR2 transmembrane domain and a TLR2 signaling domain, and the second TLR polypeptide comprises a TLR1 transmembrane domain and a TLR1 signaling domain. In some embodiments, the first TLR polypeptide comprises a TLR1 transmembrane domain and a TLR1 signaling domain, and the second TLR polypeptide comprises a TLR4 transmembrane domain and a TLR4 signaling domain; or the first TLR polypeptide comprises a TLR4 transmembrane domain and a TLR4 signaling domain, and the second TLR polypeptide comprises a TLR1 transmembrane domain and a TLR1 signaling domain. In some embodiments, the first TLR polypeptide comprises a TLR2 transmembrane domain and a TLR2 signaling domain, and the second TLR polypeptide comprises a TLR6 transmembrane domain and a TLR6 signaling domain; or the first TLR polypeptide comprises a TLR6 transmembrane domain and a TLR6 signaling domain, and the second TLR polypeptide comprises a TLR2 transmembrane domain and a TLR2 signaling domain. In some embodiments, the first TLR polypeptide comprises a TLR2 transmembrane domain and a TLR2 signaling domain, and the second TLR polypeptide comprises a TLR10 transmembrane domain and a TLR10 signaling domain; or the first TLR polypeptide comprises a TLR10 transmembrane domain and a TLR10 signaling domain, and the second TLR polypeptide comprises a TLR2 transmembrane domain and a TLR2 signaling domain. In some embodiments, the first TLR polypeptide comprises a TLR4 transmembrane domain and a TLR4 signaling domain, and the second TLR polypeptide comprises a TLR5 transmembrane domain and a TLR5 signaling domain; or the first TLR polypeptide comprises a TLR5 transmembrane domain and a TLR5 signaling domain, and the second TLR polypeptide comprises a TLR4 transmembrane domain and a TLR4 signaling domain.

Exemplary TLR molecules are listed in Table 1 below.

TABLE 1
Exemplary Toll-like receptor sequences.

| | | | Amino acid sequence | SEQ |
|---|---|---|---|---|
| | UniProt | NCBI | Transmembrane domain double underlined; TIR | ID |
| Name | ID | GeneID | domain underlined) | NO. |
| TLR1 | Q15399 | 7096 | MTSIFHFAIIFMLILQIRIQLSEESEFLVDRSKNGLIHVPKD | 30 |
| | | | LSQKTTILNISQNYISELWTSDILSLSKLRILIISHNRIQYL | |
| | | | DISVFKFNQELEYLDLSHNKLVKISCHPTVNLKHLDLSF | |
| | | | NAFDALPICKEFGNMSQLKFLGLSTTHLEKSSVLPIAHL | |
| | | | NISKVLLVLGETYGEKEDPEGLQDFNTESLHIVFPTNKEF | |
| | | | HFILDVSVKTVANLELSNIKCVLEDNKCSYFLSILAKLQT | |
| | | | NPKLSNLTLNNIETTWNSFIRILQLVWHTTVWYFSISNV | |
| | | | KLQGQLDFRDFDYSGTSLKALSIHQVVSDVFGFPQSYIY | |
| | | | EIFSNMNIKNFTVSGTRMVHMLCPSKISPFLHLDFSNNLL | |
| | | | TDTVFENCGHLTELETLILQMNQLKELSKIAEMTTQMKS | |
| | | | LQQLDISQNSVSYDEKKGDCSWTKSLLSLNMSSNILTDT | |
| | | | IFRCLPPRIKVLDLHSNKIKSIPKQVVKLEALQELNVAFN | |
| | | | SLTDLPGCGSFSSLSVLIIDHNSVSHPSADFFQSCQKMRSI | |
| | | | KAGDNPFQCTCELGEFVKNIDQVSSEVLEGWPDSYKCD | |
| | | | YPESYRGTLLKDFHMSELSCNITLLIVTIVATMLVLAVT | |
| | | | VTSLCSYLDLPWYLRMVCQWTQTRRRARNIPLEELQRN | |
| | | | LQFHAFISYSGHDSFWVKNELLPNLEKEGMQICLHERNF | |
| | | | VPGKSIVENIITCIEKSYKSIFVLSPNFVQSEWCHYELYFA | |
| | | | HHNLFHEGSNSLILILLEPIPQYSIPSSYHKLKSLMARRTY | |
| | | | LEWPKEKSKRGLFWANLRAAINIKLTEQAKK | |
| TLR2 | O60603 | 7097 | MPHTLWMVWVLGVIISLSKEESSNQASLSCDRNGICKG | 31 |
| | | | SSGSLNSIPSGLTEAVKSLDLSNNRITYISNSDLQRCVNL | |
| | | | QALVLTSNGINTIEEDSFSSLGSLEHLDLSYNYLSNLSSS | |
| | | | WFKPLSSLTFLNLLGNPYKTLGETSLFSHLTKLQILRVG | |
| | | | NMDTFTKIQRKDFAGLTFLEELEIDASDLQSYEPKSLKSI | |
| | | | QNVSHLILHMKQHILLLEIFVDVTSSVECLELRDTDLDTF | |
| | | | HFSELSTGETNSLIKKFTFRNVKITDESLFQVMKLLNQIS | |
| | | | GLLELEFDDCTLNGVGNFRASDNDRVIDPGKVETLTIRR | |
| | | | LHIPRFYLFYDLSTLYSLTERVKRITVENSKVFLVPCLLS | |
| | | | QHLKSLEYLDLSENLMVEEYLKNSACEDAWPSLQTLIL | |
| | | | RQNHLASLEKTGETLLTLKNLTNIDISKNSFHSMPETCQ | |
| | | | WPEKMKYLNLSSTRIHSVTGCIPKTLEILDVSNNNLNLFS | |
| | | | LNLPQLKELYISRNKLMTLPDASLLPMLLVLKISRNAITT | |
| | | | FSKEQLDSFHTLKTLEAGGNNFICSCEFLSFTQEQQALA | |
| | | | KVLIDWPANYLCDSPSHVRGQQVQDVRLSVSECHRTAL | |
| | | | VSGMCCALFLLILLTGVLCHRFHGLWYMKMMWAWLQ | |
| | | | AKRKPRKAPSRNICYDAFVSYSERDAYWVENLMVQEL | |
| | | | ENFNPPFKLCLHKRDFIPGKWIIDNIIDSIEKSHKTVFVLS | |
| | | | ENFVKSEWCKYELDFSHFRLFDENNDAAILILLEPIEKKA | |
| | | | IPQRFCKLRKIMNTKTYLEWPMDEAQREGFWVNLRAAI | |
| | | | KS | |
| TLR3 | O15455 | 7098 | MRQTLPCIYFWGGLLPFGMLCASSTTKCTVSHEVADCS | 32 |
| | | | HLKLTQVPDDLPTNITVLNLTHNQLRRLPAANFTRYSQL | |
| | | | TSLDVGFNTISKLEPELCQKLPMLKVLNLQHNELSQLSD | |
| | | | KTFAFCTNLTELHLMSNSIQKIKNNPFVKQKNLITLDLSH | |
| | | | NGLSSTKLGTQVQLENLQELLLSNNKIQALKSEELDIFA | |
| | | | NSSLKKLELSSNQIKEFSPGCFHAIGRLFGLFLNNVQLGP | |
| | | | SLTEKLCLELANTSIRNLSLSNSQLSTTSNTTFLGLKWTN | |
| | | | LTMLDLSYNNLNVVGNDSFAWLPQLEYFFLEYNNIQHL | |
| | | | FSHSLHGLFNVRYLNLKRSFTKQSISLASLPKIDDFSFQW | |
| | | | LKCLEHLNMEDNDIPGIKSNMFTGLINLKYLSLSNSFTSL | |
| | | | RTLTNETFVSLAHSPLHILNLTKNKISKIESDAFSWLGHL | |
| | | | EVLDLGLNEIGQELTGQEWRGLENIFEIYLSYNKYLQLT | |
| | | | RNSFALVPSLQRLMLRRVALKNVDSSPSPFQPLRNLTIL | |
| | | | DLSNNNIANINDDMLEGLEKLEILDLQHNNLARLWKHA | |
| | | | NPGGPIYFLKGLSHLHILNLESNGFDEIPVEVFKDLFELKI | |
| | | | IDLGLNNLNTLPASVENNQVSLKSLNLQKNLITSVEKKV | |
| | | | FGPAFRNLTELDMRFNPFDCTCESIAWFVNWINETHTNI | |
| | | | PELSSHYLCNTPPHYHGFPVRLFDTSSCKDSAPFELFFMI | |
| | | | NTSILLIFIFIVLLIHFEGWRISFYWNVSVHRVLGFKEIDR | |
| | | | QTEQFEYAAYIIHAYKDKDWVWEHFSSMEKEDQSLKFC | |
| | | | LEERDFEAGVFELEAIVNSIKRSRKIIFVITHHLLKDPLCK | |
| | | | RFKVHHAVQQAIEQNLDSIILVFLEEIPDYKLNHALCLRR | |
| | | | GMFKSHCILNWPVQKERIGAFRHKLQVALGSK | |
| | | | NSVH | |
| TLR4 | O00206 | 7099 | MMSASRLAGTLIPAMAFLSCVRPESWEPCVEVVPNITY | 33 |
| | | | QCMELNFYKIPDNLPFSTKNLDLSFNPLRHLGSYSFFSFP | |
| | | | ELQVLDLSRCEIQTIEDGAYQSLSHLSTLILTGNPIQSLAL | |
| | | | GAFSGLSSLQKLVAVETNLASLENFPIGHLKTLKELNVA | |
| | | | HNLIQSFKLPEYFSNLTNLEHLDLSSNKIQSIYCTDLRVL | |
| | | | HQMPLLNLSLDLSLNPMNFIQPGAFKEIRLHKLTLRNNF | |
| | | | DSLNVMKTCIQGLAGLEVHRLVLGEFRNEGNLEKFDKS | |
| | | | ALEGLCNLTIEEFRLAYLDYYLDDIIDLFNCLTNVSSFSL | |
| | | | VSVTIERVKDFSYNFGWQHLELVNCKFGQFPTLKLKSL | |
| | | | KRLTFTSNKGGNAFSEVDLPSLEFLDLSRNGLSFKGCCS | |
| | | | QSDFGTTSLKYLDLSFNGVITMSSNFLG | |
| | | | LEQLEHLDFQHSNLKQMSEFSVFLSLRNLIYLDISHTHTR | |
| | | | VAFNGIFNGLSSLEVLKMAGNSFQENFLPDIFTELRNLTF | |
| | | | LDLSQCQLEQLSPTAFNSLSSLQVLNMSHNNFFSLDTFP | |
| | | | YKCLNSLQVLDYSLNHIMTSKKQELQHFPSSLAFLNLTQ | |
| | | | NDFACTCEHQSFLQWIKDQRQLLVEVERMECATPSDKQ | |
| | | | GMPVLSLNITCQMNKTIIGVSVLSVLVVSVVAVLVYKF | |
| | | | YFHLMLLAGCIKYGRGENIYDAFVIYSSQDEDWVRNEL | |
| | | | VKNLEEGVPPFQLCLHYRDFIPGVAIAANIIHEGFHKSRK | |
| | | | VIVVVSQHFIQSRWCIFEYEIAQTWQFLSSRAGIIFIVLQK | |
| | | | VEKTLLRQQVELYRLLSRNTYLEWEDSVLGRHIFWRRL | |
| | | | RKALLDGKSWNPEGTVGTGCNWQEATSI | |
| TLR5 | O60602 | 7100 | MGDHLDLLLGVVLMAGPVFGIPSCSFDGRIAFYRFCNLT | 34 |
| | | | QVPQVLNTTERLLLSFNYIRTVTASSFPFLEQLQLLELGS | |
| | | | QYTPLTIDKEAFRNLPNLRILDLGSSKIYFLHPDAFQGLF | |
| | | | HLFELRLYFCGLSDAVLKDGYFRNLKALTRLDLSKNQIR | |
| | | | SLYLHPSFGKLNSLKSIDFSSNQIFLVCEHELEPLQGKTLS | |
| | | | FFSLAANSLYSRVSVDWGKCMNPFRNMVLEILDVSGNG | |
| | | | WTVDITGNFSNAISKSQAFSLILAHHIMGAGFGFHNIKDP | |
| | | | DQNTFAGLARSSVRHLDLSHGFVFSLNSRVFETLKDLK | |
| | | | VLNLAYNKINKIADEAFYGLDNLQVLNLSYNLLGELYS | |
| | | | SNFYGLPKVAYIDLQKNHIAIIQDQTFKFLEKLQTLDLRD | |
| | | | NALTTIHFIPSIPDIFLSGNKLVTLPKINLTANLIHLSENRL | |
| | | | ENLDILYFLLRVPHLQILILNQNRFSSCSGDQTPSENPSLE | |
| | | | QLFLGENMLQLAWETELCWDVFEGLSHLQVLYLNHNY | |
| | | | LNSLPPGVFSHLTALRGLSLNSNRLTVLSHNDLPANLEIL | |
| | | | DISRNQLLAPNPDVFVSLSVLDITHNKFICECELSTFINW | |
| | | | LNHTNVTIAGPPADIYCVYPDSFSGVSLFSLSTEGCDEEE | |
| | | | VLKSLKFSLFIVCTVTLTLFLMTILTVTKFRGFCFICYKT | |
| | | | AQRLVFKDHPQGTEPDMYKYDAYLCFSSKDFTWVQNA | |
| | | | LLKHLDTQYSDQNRFNLCFEERDFVPGENRIANIQDAIW | |
| | | | NSRKIVCLVSRHFLRDGWCLEAFSYAQGRCLSDLNSALI | |
| | | | MVVVGSLSQYQLMKHQSIRGFVQKQQYLRWPEDFQDV | |
| | | | GWFLHKLSQQILKKEKEKKKDNNIPLQTVATIS | |
| TLR6 | Q9Y2C9 | 10333 | MTKDKEPIVKSFHFVCLMIIIVGTRIQFSDGNEFAVDKSK | 35 |
| | | | RGLIHVPKDLPLKTKVLDMSQNYIAELQVSDMSFLSELT | |
| | | | VLRLSHNRIQLLDLSVFKFNQDLEYLDLSHNQLQKISCH | |
| | | | PIVSFRHLDLSFNDFKALPICKEFGNLSQLNFLGLSAMKL | |
| | | | QKLDLLPIAHLHLSYILLDLRNYYIKENETESLQILNAKT | |
| | | | LHLVFHPTSLFAIQVNISVNTLGCLQLTNIKLNDDNCQV | |
| | | | FIKFLSELTRGSTLLNFTLNHIETTWKCLVRVFQFLWPKP | |
| | | | VEYLNIYNLTIIESIREEDFTYSKTTLKALTIEHITNQVFLF | |
| | | | SQTALYTVFSEMNIMMLTISDTPFIHMLCPHAPSTFKFLN | |
| | | | FTQNVFTDSIFEKCSTLVKLETLILQKNGLKDLFKVGLM | |
| | | | TKDMPSLEILDVSWNSLESGRHKENCTWVESIVVLNLSS | |
| | | | NMLTDSVFRCLPPRIKVLDLHSNKIKSVPKQVVKLEALQ | |
| | | | ELNVAFNSLTDLPGCGSFSSLSVLIIDHNSVSHPSADFFQ | |
| | | | SCQKMRSIKAGDNPFQCTCELREFVKNIDQVSSEVLEG | |
| | | | WPDSYKCDYPESYRGSPLKDFHMSELSCNITLLIVTIGAT | |
| | | | MLVLAVTVTSLCIYLDLPWYLRMVCQWTQTRRRARNIP | |
| | | | LEELQRNLQFHAFISYSEHDSAWVKSELVPYLEKEDIQIC | |
| | | | LHERNFVPGKSIVENIINCIEKSYKSIFVLSPNFVQSEWCH | |
| | | | YELYFAHHNLFHEGSNNLILILLEPIPQNSIPNKYHKLKA | |
| | | | LMTQRTYLQWPKEKSKRGLFWANIRAAFNMKLTLVTE | |
| | | | NNDVKS | |
| TLR7 | Q9NYK1 | 51284 | MVFPMWTLKRQILILFNIILISKLLGARWFPKTLPCDVTL | 36 |
| | | | DVPKNHVIVDCTDKHLTEIPGGIPTNTTNLTLTINHIPDIS | |
| | | | PASFHRLDHLVEIDFRCNCVPIPLGSKNNMCIKRLQIKPR | |
| | | | SFSGLTYLKSLYLDGNQLLEIPQGLPPSLQLLSLEANNIFS | |
| | | | IRKENLTELANIEILYLGQNCYYRNPCYVSYSIEKDAFLN | |
| | | | LTKLKVLSLKDNNVTAVPTVLPSTLTELYLYNNMIAKIQ | |
| | | | EDDFNNLNQLQILDLSGNCPRCYNAPFPCAPCKNNSPLQ | |
| | | | IPVNAFDALTELKVLRLHSNSLQHVPPRWFKNINKLQEL | |
| | | | DLSQNFLAKEIGDAKFLHFLPSLIQLDLSFNFELQVYRAS | |
| | | | MNLSQAFSSLKSLKILRIRGYVFKELKSFNLSPLHNLQNL | |
| | | | EVLDLGTNFIKIANLSMFKQFKRLKVIDLSVNKISPSGDS | |
| | | | SEVGFCSNARTSVESYEPQVLEQLHYFRYDKYARSCRF | |
| | | | KNKEASFMSVNESCYKYGQTLDLSKNSIFFVKSSDFQHL | |
| | | | SFLKCLNLSGNLISQTLNGSEFQPLAELRYLDFSNNRLDL | |
| | | | LHSTAFEELHKLEVLDISSNSHYFQSEGITHMLNFTKNL | |
| | | | KVLQKLMMNDNDISSSTSRTMESESLRTLEFRGNHLDV | |
| | | | LWREGDNRYLQLFKNLLKLEELDISKNSLSFLPSGVFDG | |
| | | | MPPNLKNLSLAKNGLKSFSWKKLQCLKNLETLDLSHNQ | |
| | | | LTTVPERLSNCSRSLKNLILKNNQIRSLTKYFLQDAFQLR | |
| | | | YLDLSSNKIQMIQKTSFPENVLNNLKMLLLHHNRFLCTC | |
| | | | DAVWFVWWVNHTEVTIPYLATDVTCVGPGAHKGQSVI | |
| | | | SLDLYTCELDLTNLILFSLSISVSLFLMVMMTASHLYFW | |
| | | | DVWYIYHFCKAKIKGYQRLISPDCCYDAFIVYDTKDPA | |
| | | | VTEWVLAELVAKLEDPREKHFNLCLEERDWLPGQPVLE | |
| | | | NLSQSIQLSKKTVFVMTDKYAKTENFKIAFYLSHQRLM | |
| | | | DEKVDVIILIFLEKPFQKSKFLQLRKRLCGSSVLEWPTNP | |
| | | | QAHPYFWQCLKNALATDNHVAYSQVFKETV | |
| TLR8 | Q9NR97 | 51311 | VGKYVTELDLSDNFITHITNESFQGLQNLTKINLNHNPN | 37 |
| | | | VQHQNGNPGIQSNGLNITDGAFLNLKNLRELLLEDNQLP | |
| | | | QIPSGLPESLTELSLIQNNIYNITKEGISRLINLKNLYLAW | |
| | | | NCYFNKVCEKTNIEDGVFETLTNLELLSLSFNSLSHVPPK | |
| | | | LPSSLRKLFLSNTQIKYISEEDFKGLINLTLLDLSGNCPRC | |
| | | | FNAPFPCVPCDGGASINIDRFAFQNLTQLRYLNLSSTSLR | |
| | | | KINAAWFKNMPHLKVLDLEFNYLVGEIASGAFLTMLPR | |
| | | | LEILDLSFNYIKGSYPQHINISRNFSKLLSLRALHLRGYVF | |
| | | | QELREDDFQPLMQLPNLSTINLGINFIKQIDFKLFQNFSN | |
| | | | LEIIYLSENRISPLVKDTRQSYANSSSFQRHIRKRRSTDFE | |
| | | | FDPHSNFYHFTRPLIKPQCAAYGKALDLSLNSIFFIGPNQ | |
| | | | FENLPDIACLNLSANSNAQVLSGTEFSAIPHVKYLDLTN | |
| | | | NRLDFDNASALTELSDLEVLDLSYNSHYFRIAGVTHHLE | |
| | | | FIQNFTNLKVLNLSHNNIYTLTDKYNLESKSLVELVFSG | |
| | | | NRLDILWNDDDNRYISIFKGLKNLTRLDLSLNRLKHIPN | |
| | | | EAFLNLPASLTELHINDNMLKFFNWTLLQQFPRLELLDL | |
| | | | RGNKLLFLTDSLSDFTSSLRTLLLSHNRISHLPSGFLSEVS | |
| | | | SLKHLDLSSNLLKTINKSALETKTTTKLSMLELHGNPFE | |
| | | | CTCDIGDFRRWMDEHLNVKIPRLVDVICASPGDQRGKSI | |
| | | | VSLELTTCVSDVTAVILFFFTFFITTMVMLAALAHHLFY | |
| | | | WDVWFIYNVCLAKVKGYRSLSTSQTFYDAYISYDTKDA | |
| | | | SVTDWVINELRYHLEESRDKNVLLCLEERDWDPGLAIID | |
| | | | NLMQSINQSKKTVFVLTKKYAKSWNFKTAF | |
| | | | YLALQRLMDENMDVIIFILLEPVLQHSQYLRLRQRICKSS | |
| | | | ILQWPDNPKAEGLFWQTLRNVVLTENDSRYNNMYVDSI | |
| | | | KQY | |
| TLR9 | Q9NR96 | 54106 | MGFCRSALHPLSLLVQAIMLAMTLALGTLPAFLPCELQP | 38 |
| | | | HGLVNCNWLFLKSVPHFSMAAPRGNVTSLSLSSNRIHH | |
| | | | LHDSDFAHLPSLRHLNLKWNCPPVGLSPMHFPCHMTIEP | |
| | | | STFLAVPTLEELNLSYNNIMTVPALPKSLISLSLSHTNILM | |
| | | | LDSASLAGLHALRFLFMDGNCYYKNPCRQALEVAPGA | |
| | | | LLGLGNLTHLSLKYNNLTVVPRNLPSSLEYLLLSYNRIV | |
| | | | KLAPEDLANLTALRVLDVGGNCRRCDHAPNPCMECPR | |
| | | | HFPQLHPDTFSHLSRLEGLVLKDSSLSWLNASWFRGLG | |
| | | | NLRVLDLSENFLYKCITKTKAFQGLTQLRKLNLSFNYQK | |
| | | | RVSFAHLSLAPSFGSLVALKELDMHGIFFRSLDETTLRPL | |
| | | | ARLPMLQTLRLQMNFINQAQLGIFRAFPGLRYVDLSDN | |
| | | | RISGASELTATMGEADGGEKVWLQPGDLAPAPVDTPSS | |
| | | | EDFRPNCSTLNFTLDLSRNNLVTVQPEMFAQLSHLQCLR | |
| | | | LSHNCISQAVNGSQFLPLTGLQVLDLSHNKLDLYHEHSF | |
| | | | TELPRLEALDLSYNSQPFGMQGVGHNFSFVAHLRTLRH | |
| | | | LSLAHNNIHSQVSQQLCSTSLRALDFSGNALGHMWAEG | |
| | | | DLYLHFFQGLSGLIWLDLSQNRLHTLLPQTLRNLPKSLQ | |
| | | | VLRLRDNYLAFFKWWSLHFLPKLEVLDLAGNQLKALT | |
| | | | NGSLPAGTRLRRLDVSCNSISFVAPGFFSKAKELRELNLS | |
| | | | ANALKTVDHSWFGPLASALQILDVSANPLHCACGAAF | |
| | | | MDFLLEVQAAVPGLPSRVKCGSPGQLQGLSIFAQDLRL | |
| | | | CLDEALSWDCFALSLLAVALGLGVPMLHHLCGWDLW | |
| | | | YCFHLCLAWLPWRGRQSGRDEDALPYDAFVVEDKTQS | |
| | | | AVADWVYNELRGQLEECRGRWALRLCLEERDWLPGK | |
| | | | TLFENLWASVYGSRKTLFVLAHTDRVSGLLRASFLLAQ | |
| | | | QRLLEDRKDVVVLVILSPDGRRSRYVRLRQRLCRQSVL | |
| | | | LWPHQPSGQRSFWAQLGMALTRDNHHFYNRNFCQGPT | |
| | | | AE | |
| TLR10 | Q9BXR5 | 81793 | MRLIRNIYIFCSIVMTAEGDAPELPEERELMTNCSNMSLR | 39 |
| | | | KVPADLTPATTTLDLSYNLLFQLQSSDFHSVSKLRVLILC | |
| | | | HNRIQQLDLKTFEFNKELRYLDLSNNRLKSVTWYLLAG | |
| | | | LRYLDLSFNDFDTMPICEEAGNMSHLEILGLSGAKIQKS | |
| | | | DFQKIAHLHLNTVFLGFRTLPHYEEGSLPILNTTKLHIVL | |
| | | | PMDTNFWVLLRDGIKTSKILEMTNIDGKSQFVSYEMQR | |
| | | | NLSLENAKTSVLLLNKVDLLWDDLFLILQFVWHTSVEH | |
| | | | FQIRNVTFGGKAYLDHNSFDYSNTVMRTIKLEHVHFRV | |
| | | | FYIQQDKIYLLLTKMDIENLTISNAQMPHMLFPNYPTKF | |
| | | | QYLNFANNILTDELFKRTIQLPHLKTLILNGNKLETLSLV | |
| | | | SCFANNTPLEHLDLSQNLLQHKNDENCSWPETVVNMN | |
| | | | LSYNKLSDSVFRCLPKSIQILDLNNNQIQTVPKETIHLMA | |
| | | | LRELNIAFNFLTDLPGCSHFSRLSVLNIEMNFILSPSLDFV | |
| | | | QSCQEVKTLNAGRNPFRCTCELKNFIQLETYSEVMMVG | |
| | | | WSDSYTCEYPLNLRGTRLKDVHLHELSCNTALLIVTIVV | |
| | | | IMLVLGLAVAFCCLHFDLPWYLRMLGQCTQTWHRVRK | |
| | | | TTQEQLKRNVRFHAFISYSEHDSLWVKNELIPNLEKEDG | |
| | | | SILICLYESYFDPGKSISENIVSFIEKSYKSIFVLSPNFVQN | |

EWCHYEFYFAHHNLFHENSDHIILILLEPIPFYCIPTRYHK
LKALLEKKAYLEWPKDRRKCGLFWANLRAAINVNVLA
TREMYELQTFTELNEESRGSTISLMRTDCL

In some embodiments, the TLR polypeptides induce increased cytoxicity against target cells by the modified immune cell, such as increased by at least about any one of 10%, 20%, 30%, 40%, 2 fold, 4 fold, 6 fold, or more, compared to a modified immune cell not comprising said TLR receptor domains. In some embodiments, the TLR receptor domains induce increased cytoxicity against target cells by the modified immune cell, such as increased by nor more than about any one of 6 fold, 4 fold, 2 fold, 40%, 30%, 20%, 10%, or less, compared to a modified immune cell not comprising said TLR receptor domains. In some embodiments, cytoxicity is measured by Lactate dehydrogenase (LDH) cytoxicity assays and/or co-culture assays with target tumor cells. In some embodiments, the cytoxicity and tumor cell killing capability is measured in a cell-based assay. In some embodiments, the cytoxicity and tumor cell killing capability are measured in vivo.

In some embodiments, the TLR polypeptides induce a reduced level of inflammatory cytokine secretion by the modified immune cell such as reduced by at least about any one of 10%, 20%, 30%, 40%, 2 fold, 4 fold, 6 fold, 8 fold, 10 fold, 12 fold, 14 fold, 16 fold, 18 fold, 20 fold, 30 fold, 50 fold, 100 fold, 200 fold, 500 fold, 1000 fold, or more, compared to a modified immune cell not comprising said TLR receptor domains. In some embodiments, the TLR polypeptides induce a reduced level of inflammatory cytokine secretion by the modified immune cell, such as reduced by no more than about any one of 1000 fold, 500 fold, 200 fold, 100 fold, 50 fold, 30 fold, 20 fold, 18 fold, 16 fold, 14 fold, 12 fold, 10 fold, 8 fold, 6 fold, 4 fold, 50%, 40%, 30%, 20%, 10% or less, compared to a modified immune cell not comprising said TLR polypeptides. In some embodiments, the TLR polypeptides induce a reduced level of inflammatory cytokine secretion by the modified immune cell, such as reduced by between about any of 10%-50%, 2-1000 fold, 2-50 fold, 50-100 fold, 100-1000 fold, 50-500 fold, 10-100 fold, 10-50 fold, or 50-200 fold, compared to a modified immune cell not comprising said TLR polypeptides. Exemplary inflammatory cytokines include, but are not limited to, e.g., IFN-γ, TNF-α, and GM-CSF. In some embodiments, secretion levels of inflammatory cytokines are measured by in serum immunoassays, such as enzyme-linked immunosorbent assay (ELISA), chemiluminescent immunoassays (CIA), or flow cytometry. In some embodiments, the inflammatory cytokine secretion levels are measured in a cell-based assay. In some embodiments, the inflammatory cytokine secretion levels are measured in vivo.

In some embodiments, the TLR polypeptide is a co-stimulatory molecule. In some embodiments, the TLR polypeptide co-stimulatory molecule is an inducible co-stimulatory molecule, wherein upon binding of a first target binding domain and a second target binding domain to their corresponding target, a first TLR signaling domain and a second TLR signaling domain associate with each other to form a TLR signaling moiety capable of inducing TLR signaling.

In some embodiments, the inducible co-stimulatory molecule is connected to an engineered receptor (e.g., any of the engineered receptors described herein) via a self-cleaving peptide, such as P2A. In some embodiments, the engineered receptor is a chimeric antigen receptor (CAR). In some embodiments, the TLR polypeptide comprises a transmembrane domain and a signaling domain. In some embodiments, the TLR polypeptide is a co-stimulatory molecule and comprises a TLR transmembrane domain and a TLR signaling domain. In some embodiments, the signaling domain is an intracellular domain, such as an intracellular signaling domain. In some embodiments, the TLR polypeptide comprises: (a) target binding domain, (b) a transmembrane domain; and (c) a signaling domain (e.g., an intracellular signaling domain). Optionally, the TLR polypeptide further comprises a signaling peptide.

In some embodiments, the TLR polypeptide comprises a transmembrane domain that is directly or indirectly fused to the TLR signaling domain. In some embodiments, the TLR polypeptide comprises a transmembrane domain that is directly or indirectly fused to a target binding domain (e.g., a first target binding domain and/or a second target binding domain). In some embodiments, TLR polypeptide comprises a transmembrane domain that is directly or indirectly fused to both a target binding domain and a TLR signaling domain. In some embodiments, the TLR polypeptide comprises a TLR transmembrane domain from a TLR molecule. In some embodiments, the TLR molecule is selected from the group consisting of TLR1, TLR2, TLR3, TLR4, TLR5, TLR6, TLR7, TLR8, TLR9, and TLR10. In some embodiments, the TLR transmembrane domain is derived from TLR1. In some embodiments, the TLR transmembrane domain is derived from TLR2. In some embodiments, the TLR transmembrane domain is derived from TLR4. In some embodiments, the TLR transmembrane domain is derived from TLR6.

In some embodiments, the modified immune cell comprises a first polypeptide and a second polypeptide. In some embodiments, the first polypeptide comprises a first target binding domain, a first TLR transmembrane domain, and a first TLR signaling domain. In some embodiments, the second polypeptide comprises a second target binding domain, a second TLR transmembrane domain, and a second TLR signaling domain.

In some embodiments, the first TLR transmembrane domain and the first TLR signaling domain are derived from the same TLR molecule. In some embodiments, the first TLR transmembrane domain and/or the first TLR signaling domain are derived from the group consisting of TLR1, TLR2, TLR3, TLR4, TLR5, TLR6, TLR7, TLR8, TLR9, and TLR10. In some embodiments, the first TLR transmembrane domain and/or the first TLR signaling domain are derived from the group consisting of TLR1, TLR2, TLR4, and TLR6. In some embodiments, the first TLR transmembrane domain and/or the first TLR signaling domain are derived from TLR1. In some embodiments, the first TLR transmembrane domain and/or the first TLR signaling domain are derived from TLR4.

In some embodiments, the second TLR transmembrane domain and the second TLR signaling domain are derived from the same TLR molecule. In some embodiments, the second TLR transmembrane domain and/or the second TLR signaling domain are derived from the group consisting of TLR1, TLR2, TLR3, TLR4, TLR5, TLR6, TLR7, TLR8, TLR9, and TLR10. In some embodiments, the second TLR transmembrane domain and/or the second TLR signaling domain are derived from the group consisting of TLR1, TLR2, TLR4, and TLR6. In some embodiments, the second TLR transmembrane domain and/or the first TLR signaling domain are derived from TLR2.

In some embodiments, the first TLR transmembrane domain and the second TLR transmembrane domain are the same. In some embodiments, the first TLR transmembrane domain is derived from TLR1, and the second TLR transmembrane domain is derived from TLR1. In some embodiments, the first TLR transmembrane domain is derived from TLR2, and the second TLR transmembrane domain is derived from TLR2. In some embodiments, the first TLR transmembrane domain is derived from TLR4, and the second TLR transmembrane domain is derived from TLR4. In some embodiments, the first TLR transmembrane domain is derived from TLR6, and the second TLR transmembrane domain is derived from TLR6.

In some embodiments, the first TLR signaling domain and the second TLR signaling domain are the same. In some embodiments, the first TLR signaling domain is derived from TLR1, and the second TLR signaling domain is derived from TLR1. In some embodiments, the first TLR signaling domain is derived from TLR2, and the second TLR signaling domain is derived from TLR2. In some embodiments, the first TLR signaling domain is derived from TLR4, and the second TLR signaling domain is derived from TLR4. In some embodiments, the first TLR signaling domain is derived from TLR6, and the second TLR signaling domain is derived from TLR6.

In some embodiments, the first TLR transmembrane domain and the second TLR transmembrane domain are different. In some embodiments, the first TLR transmembrane domain is derived from TLR2, and the second TLR transmembrane domain is derived from TLR1. In some embodiments, the first TLR signaling domain and the second TLR signaling domain are different. In some embodiments, the first TLR signaling domain is derived from TLR2, and the second TLR signaling domain is derived from TLR1. In some embodiments, the first TLR transmembrane domain is derived from TLR2, the first TLR signaling domain is derived from TLR2, the second TLR transmembrane domain is derived from TLR1, and the second TLR signaling domain is derived from TLR1.

In some embodiments, the TLR polypeptides described herein comprise a target binding domain. In some embodiments, the target binding domain is not derived from a receptor or a ligand. In some embodiments, the first polypeptide and the second polypeptide are not derived from TGFβ. In some embodiments, the first polypeptide and the second polypeptide are not TGFβ. In some embodiments, the first polypeptide and the second polypeptide do not comprise TGFβ extracellular domains. In some embodiments, the first polypeptide and the second polypeptide do not comprise an extracellular TGFβ-binding domain of a TGFβ (e.g., TGFβR1 and/or TGFβR2). In some embodiments, the target binding domain does not comprise the extracellular TGFβ-binding domain of TGFβR1. In some embodiments, the target binding domain does not comprise the extracellular TGFβ-binding domain of TGFβR2. In some embodiments, the target binding domain is not an extracellular TGFβ-binding domain. In some embodiments, the target binding domain is not the extracellular TGFβ-binding domain of TGFβR2. In some embodiments, the target binding domain is not the extracellular TGFβ-binding domain of TGFβR1. In some embodiments, the TLR polypeptide (e.g., the first polypeptide and/or the second polypeptide) is not a chimeric TGFβ receptor CTBR signal converter described in WO2018/094244A1, which is incorporated herein by reference in its entirety.

In some embodiments, the modified immune cells provided herein comprise a first and second TLR polypeptide, wherein the first TLR polypeptide comprises a first target binding domain and the second TLR polypeptide comprises a second target binding domain. In some embodiments, the first target binding domain and the second target binding domain are the same. In some embodiments, the first target binding domain and the second target binding domain are different.

In some embodiments, the target binding domain of the TLR polypeptide is an antibody or an antibody fragment, such as a scFv, a Fv, a Fab, a (Fab′)₂, a single domain antibody (sdAb), or a V_{H}H domain. In some embodiments, the polypeptide is a ligand or an extracellular portion of a receptor that to a target molecule. In some embodiments, the target molecule is a tumor antigen. In some embodiments, the target binding domain of the TLR polypeptide specifically binds to a single tumor antigen. In some embodiments, the tumor antigen is selected from the group consisting of CD19, BCMA, NY-ESO-1, VEGFR2, MAGE-A3, CD20, CD22, CD33, CLL1, CD38, CEA, EGFR (such as EGFRvIII), GD2, HER2, IGF1R, mesothelin, PSMA, ROR1, WT1, and other tumor antigens with clinical significance, and combinations thereof. In some embodiments, the TLR polypeptide specifically binds to a target antigen selected from the group consisting of NKG2D, GPRC5D, BCMA, NY-ESO-1, VEGFR2, MAGE-A3, AFP, CD4, CD19, CD20, CD22, CD30, CD33, CD38, CD70, CD123, CEA, EGFR (such as EGFRvIII), GD2, GPC-2, GPC3, CLDN18.2, HER2, LILRB4, IL-13Rα2, IGF1R, mesothelin, PSMA, ROR1, WT1, NKG2D, CLL1, TGFaRII, TGFbRII, CCR5, CXCR4, CCR4, HPV related antigens, and EBV related antigens (e.g., LMP1 or LMP2). In some embodiments, the target binding domain specifically binds to CD33, CLL1, CD123, CD19, CD20, CD22, BCMA, NKG2D, GPRC5D, or GPC3.

In some embodiments, the target molecule is an immune checkpoint protein. In some embodiments, the target molecule is selected from the group consisting of PD-1, CD70, CD27, SIRPα, and TIGIT.

In some embodiments, the target molecule is a natural protein expressed on immune cells. In some embodiments, the target molecule is NKG2D. In some embodiments, the target molecule is mutated NKG2D. In some embodiments, the mutated NKG2D comprises a truncated sequence and/or an amino acid substitution, mutation, addition, and/or deletion. In some embodiments, the target molecule is an extracellular antigen binding domain of NKG2D (also referred to herein as “NKG2D ECD”). In some embodiments, the target molecule is full-length sequence of NKG2D. In some embodiments, the NKG2D comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 54. In some embodiments the second polypeptide comprises SEQ ID NO: 54. In some embodiments, the NKG2D comprises the amino acids at positions 81-216 of the full-length sequence of the ECD of NKG2D (SEQ ID NO: 54). In some embodiments, the NKG2D comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 51. In some embodiments the second polypeptide comprises SEQ ID NO: 51. In some embodiments, the NKG2D comprises the amino acids at positions 89-216 of the full-length sequence of the ECD of NKG2D (SEQ ID NO: 54). In some embodiments, the NKG2D comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 52. In some embodiments the second polypeptide comprises SEQ ID NO: 52. In some embodiments, the NKG2D comprises the amino acids at positions 98-216 of the full-length sequence of the ECD of NKG2D (SEQ ID NO: 54). In some embodiments, the NKG2D comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 53. In some embodiments the second polypeptide comprises SEQ ID NO: 53.

In some embodiments, the TLR polypeptide further comprises a signal peptide that targets the TLR polypeptide to the secretory pathway of the cell (e.g., ER) and will allow for integration and anchoring of the TLR polypeptide into the lipid bilayer of the host cell. Signal peptides including signal sequences of naturally occurring proteins or synthetic, non-naturally occurring signal sequences, which are compatible for use in the TLR polypeptides described herein will be evident to one of skill in the art. In some embodiments, the signal peptide is derived from a molecule selected from the group consisting of CD8α, GM-CSF receptor α, TL-3, and IgG1 heavy chain. In some embodiments, the signal peptide is derived from CD8α.

The TLR polypeptide may comprise one or more peptide linkers disposed between different domains. For example, the target binding domain and the TLR transmembrane domain and/or the TLR transmembrane domain and the TLR signaling domain can be fused to each other via a peptide bond or via a peptide linker. The peptide linkers connecting different domains may be the same or different. Each peptide linker can be optimized individually. The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50 or more amino acids long. In some embodiments, the peptide linker is no more than about any of 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, or about 1 amino acid to about 50 amino acids.

The peptide linker may have a naturally occurring sequence, or a non-naturally occurring sequence. In some embodiments, the peptide linker is a flexible linker. Exemplary flexible linkers include glycine polymers (G)ₙ, glycine-serine polymers (including, for example, (GS)ₙ(SEQ ID NO: 27), (GSGGS)ₙ(SEQ ID NO: 28) and (GGGS)ₙ(SEQ ID NO: 29), where n is an integer of at least one), glycine-alanine polymers, alanine-serine polymers, and other flexible linkers known in the art. In some embodiments, the peptide linker has the amino acid sequence of SEQ ID NO: 25 or 26.

In some embodiments, the TLR polypeptide comprises a transmembrane domain and a signaling domain derived from the same TLR molecule. In some embodiments, the TLR polypeptide comprises a TLR1 transmembrane domain and a TLR1 signaling domain. In some embodiments, the TLR polypeptide comprises a TLR2 transmembrane domain and a TLR2 signaling domain. In some embodiments, the TLR polypeptide comprises a TLR4 transmembrane domain and a TLR4 signaling domain. In some embodiments, the TLR polypeptide comprises a TLR6 transmembrane domain and a TLR6 signaling domain. In some embodiments, the TLR polypeptide further comprises a signal peptide (e.g., leader sequence). In some embodiments, the TLR polypeptide further comprises an intracellular domain of a cytokine receptor.

In some embodiments, the TLR polypeptide comprising a transmembrane domain and a signaling domain derived from the same TLR molecule further comprises a target binding domain. In some embodiments, the target binding domain is a scFv or sdAb that specifically binds to NKG2D, CD33, CLL1, CD123, CD19, CD20, CD22, BCMA, GPRC5D, or GPC3. In some embodiments, the TLR polypeptide comprising a transmembrane domain and a signaling domain derived from the same TLR molecule further comprises a target binding domain that specifically binds to CD33. In some embodiments, the TLR polypeptide comprising a transmembrane domain and a signaling domain derived from the same TLR molecule further comprises a target binding domain that specifically binds to the V domain of CD33. In some embodiments, the TLR polypeptide comprises a TLR2 transmembrane domain, a TLR2 signaling domain derived, and a target binding domain that specifically binds to the V domain of CD33. In some embodiments, the TLR polypeptide comprising a transmembrane domain and a signaling domain derived from the same TLR molecule further comprises a target binding domain that specifically binds to the C2 domain of CD33. In some embodiments, the TLR polypeptide comprises a TLR1 transmembrane domain, a TLR1 signaling domain derived, and a target binding domain that specifically binds to the C2 domain of CD33. In some embodiments, the TLR polypeptide comprises a transmembrane domain and a signaling domain derived from the same TLR molecule further comprises a target binding domain that specifically binds to CD20. In some embodiments, the TLR polypeptide comprises a TLR4 transmembrane domain, a TLR4 signaling domain derived, and a target binding domain that specifically binds to CD20. In some embodiments, the TLR polypeptide comprising a transmembrane domain and a signaling domain derived from the same TLR molecule further comprises a target binding domain that specifically binds to CD33. In some embodiments, the TLR polypeptide comprises a TLR4 transmembrane domain, a TLR4 signaling domain derived, and a target binding domain that specifically binds to CD33. In some embodiments, the TLR polypeptide comprises a transmembrane domain and a signaling domain derived from the same TLR molecule further comprises a target binding domain that specifically binds to NKG2D. In some embodiments, the TLR polypeptide comprises a TLR4 transmembrane domain, a TLR4 signaling domain derived, and a target binding domain that specifically binds to NKG2D. In some embodiments, the TLR polypeptide comprises a transmembrane domain and a signaling domain derived from the same TLR molecule further comprises a target binding domain that specifically binds to BCMA. In some embodiments, the TLR polypeptide comprises a TLR4 transmembrane domain, a TLR4 signaling domain derived, and a target binding domain that specifically binds to BCMA. In some embodiments, the TLR polypeptide comprises a transmembrane domain and a signaling domain derived from the same TLR molecule further comprises a target binding domain that specifically binds to GPRC5D. In some embodiments, the TLR polypeptide comprises a TLR4 transmembrane domain, a TLR4 signaling domain derived, and a target binding domain that specifically binds to GPRC5D. In some embodiments, the TLR polypeptide further comprises a signal peptide (e.g., a leader sequence).

In some embodiments, the TLR polypeptide further comprises an intracellular domain of a cytokine receptor. In some embodiments, the TLR polypeptide comprises two or more intraceullar domains of cytokine receptor(s). In some embodiments, the C-terminus of the intraceullar domain of the cytokine receptor is fused to the N-terminus of the TLR signaling domain. In some embodiments, the C-terminus of the intraceullar domain of the cytokine receptor is directly fused to the N-terminus of the TLR domain. In some embodiments, the C-terminus of the intraceullar domain of the cytokine receptor is indirectly fused to the N-terminus of the TLR domain, such as via a linker (e.g., via a flexible peptide linker) or via another domain. In some embodiments, the TLR polypeptide comprises from the N-terminus to the C-terminus: i) a target binding domain, ii) an intracellular domain of a cytokine receptor, iii) TLR transmembrane domain, and iv) a TLR signaling domain.

In some embodiments, the N-terminus of the intraceullar domain of the cytokine receptor is fused to the C-terminus of the TLR signaling domain. In some embodiments, the N-terminus of the intraceullar domain of the cytokine receptor is directly fused to the C-terminus of the TLR signaling domain. In some embodiments, the N-terminus of the intraceullar domain of the cytokine receptor is indirectly fused to the C-terminus of the TLR signaling domain, such via a linker (e.g., via a flexible peptide linker) or via another domain. In some embodiments, the TLR polypeptide comprises from the N-terminus to the C-terminus: i) a target binding domain, ii) TLR transmembrane domain, iii) a TLR signaling domain, and iv) an intracellular domain of a cytokine receptor.

In some embodiments, the cytokine receptor-derived intracellular domain confers improved TLR signaling (e.g., anti-tumor effects) of the TLR polypeptide(s). In some embodiments, the cytokine receptor-derived intracellular domain is selected from group consisting of a GM-CSF receptor, an IL-18 receptor, an IL-21 receptor, an IL-15 receptor, and an IL-23 receptor. In some embodiments, the cytokine receptor-derived intracellular domain comprises an immunoreceptor tyrosine-based activation motif (ITAM).

The modified immune cells described herein comprise a first TLR polypeptide and a second TLR polypeptide. In some embodiments, the first TLR polypeptide further comprises a first intracellular domain of a first cytokine receptor. In some embodiments, the second polypeptide further comprises a second intracellular domain of a second cytokine receptor. In some embodiments, the first polypeptide further comprises a first intracellular domain of a first cytokine receptor and the second polypeptide further comprises a second intracellular domain of a second cytokine receptor. In some embodiments, the first intracellular domain of a first cytokine receptor and the second intracellular domain of a second cytokine receptor are the same. In some embodiments, the first intracellular domain of a first cytokine receptor and the second intracellular domain of a second cytokine receptor are different. In some embodiments, the first cytokine receptor is selected from the group consisting of a GM-CSF receptor, an IL-18 receptor, an IL-21 receptor, an IL-15 receptor, and an IL-23 receptor. In some embodiments, the second cytokine receptor is selected from the group consisting of a GM-CSF receptor, an IL-18 receptor, an IL-21 receptor, an IL-15 receptor, and an IL-23 receptor. In some embodiments, the first cytokine receptor and the second cytokine receptor are each selected from the group consisting of a GM-CSF receptor, an IL-18 receptor, an IL-21 receptor, an IL-15 receptor, and an IL-23 receptor. In some embodiments, the first intracellular domain of the first cytokine receptor comprise an immunoreceptor tyrosine-based activation motif (ITAM). In some embodiments, the second intracellular domain of the second cytokine receptor comprise an immunoreceptor tyrosine-based activation motif (ITAM). In some embodiments, the first intracellular domain of the first cytokine receptor and the second intracellular domain of the second cytokine receptor comprise an immunoreceptor tyrosine-based activation motif (ITAM). In some embodiments, the C-terminus of the first intracellular domain of the first cytokine receptor is fused to the N-terminus of the first TLR signaling domain. In some embodiments, the C-terminus of the second intracellular domain of the second cytokine receptor is fused to the N-terminus of the second TLR signaling domain. In some embodiments, the C-terminus of the first intracellular domain of the first cytokine receptor is fused to the N-terminus of the first TLR signaling domain; and, the C-terminus of the second intracellular domain of the second cytokine receptor is fused to the N-terminus of the second TLR signaling domain. In some embodiments, the N-terminus of the first intracellular domain of the first cytokine receptor is fused to the C-terminus of the TLR signaling domain. In some embodiments, the N-terminus of the second intracellular domain of the second cytokine receptor is fused to the C-terminus of the TLR signaling domain. In some embodiments, the N-terminus of the first intracellular domain of the first cytokine receptor is fused to the C-terminus of the TLR signaling domain; and, the N-terminus of the second intracellular domain of the second cytokine receptor is fused to the C-terminus of the TLR signaling domain.

In some embodiments, the TLR polypeptides described herein are comprised in a modified immune cell. In some embodiments, the modified immune cell comprises a CAR system (e.g., a CAR fusion construct), wherein the CAR fusion construct comprises an anti-CD19 CAR molecule and anti-CD20 TLR polypeptides. In some embodiments, each of the first polypeptide and the second polypeptide comprises from the N-terminus to the C-terminus: a signal peptide, an anti-CD20 scFv, a TLR4 transmembrane domain, and a TLR4 signaling domain (e.g., the cytoplasmic portion of TLR4). In some embodiments, the modified immune cell expresses an anti-CD19 CAR. In some embodiments, the anti-CD19 CAR comprises from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-CD19 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 2. In some embodiments, the anti-CD19 CAR comprises SEQ ID NO: 2. In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD20 scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1. In some embodiments the CAR fusion construct comprises SEQ ID NO: 1. In some embodiments, the TLR polypeptide coding sequence lacks the sequences of some or all of the putative upstream start codons. In some embodiments, the TLR polypeptide may comprise certain amino acid mutations without effect on the association between a first TLR signaling domain and a second TLR signaling domain to form a TLR signaling moiety, thereby without effect on the induction of TLR signaling.

In some embodiments, the modified immune cell comprises a CAR system (e.g., a CAR fusion construct), wherein the CAR fusion construct comprises an anti-CD19 CAR molecule and anti-NKG2D TLR polypeptides. In some embodiments, each of the first polypeptide and the second polypeptide comprises from the N-terminus to the C-terminus: a signal peptide, an anti-NKG2D ECD, a TLR4 transmembrane domain, and a TLR4 signaling domain (e.g., the cytoplasmic portion of TLR4). In some embodiments, the modified immune cell expresses an anti-CD19 CAR. In some embodiments, the anti-CD19 CAR comprises from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-CD19 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 2. In some embodiments, the anti-CD19 CAR comprises SEQ ID NO: 2. In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 59. In some embodiments the CAR fusion construct comprises SEQ ID NO: 59. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 60. In some embodiments the CAR fusion construct comprises SEQ ID NO: 60. In some embodiments, the TLR polypeptide coding sequence lacks the sequences of some or all of the putative upstream start codons. In some embodiments, the TLR polypeptide may comprise certain amino acid mutations without effect on the association between a first TLR signaling domain and a second TLR signaling domain to form a TLR signaling moiety, thereby without effect on the induction of TLR signaling.

In some embodiments, the modified immune cell comprises a CAR system (e.g., a CAR fusion construct), wherein the CAR fusion construct comprises an anti-GPC3 CAR molecule and anti-NKG2D TLR polypeptides. In some embodiments, each of the first polypeptide and the second polypeptide comprises from the N-terminus to the C-terminus: a signal peptide, an anti-NKG2D ECD, a TLR4 transmembrane domain, and a TLR4 signaling domain (e.g., the cytoplasmic portion of TLR4). In some embodiments, the modified immune cell expresses an anti-GPC3 CAR. In some embodiments, the anti-GPC3 CAR comprises from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-GPC3 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 56. In some embodiments, the anti-GPC3 CAR comprises SEQ ID NO: 56. In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 57. In some embodiments the CAR fusion construct comprises SEQ ID NO: 57. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 58. In some embodiments the CAR fusion construct comprises SEQ ID NO: 58. In some embodiments, the TLR polypeptide coding sequence lacks the sequences of some or all of the putative upstream start codons. In some embodiments, the TLR polypeptide may comprise certain amino acid mutations without effect on the association between a first TLR signaling domain and a second TLR signaling domain to form a TLR signaling moiety, thereby without effect on the induction of TLR signaling.

In some embodiments, the TLR polypeptides described herein are comprised in a modified immune cell. In some embodiments, the modified immune cell comprises a CAR system (e.g., a CAR fusion construct), wherein the CAR fusion construct comprises an anti-CLL1 CAR molecule and anti-CD33 TLR polypeptides. In some embodiments, each of the first polypeptide and the second polypeptide comprises from the N-terminus to the C-terminus: a signal peptide, an anti-CD33 sdAb, a TLR4 transmembrane domain, and a TLR4 signaling domain (e.g., the cytoplasmic portion of TLR4). In some embodiments, the modified immune cell expresses an anti-CLL1 CAR. In some embodiments, the anti-CLL1 CAR comprises from the N-terminus to the C-terminus: a signal peptide, an anti-CLL1 sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the CAR fusion construct comprises, from the N-terminus to the C-terminus: a signal peptide, an anti-CLL1 sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD33 sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 71. In some embodiments the CAR fusion construct comprises SEQ ID NO: 71. In some embodiments, the TLR polypeptide coding sequence lacks the sequences of some or all of the putative upstream start codons. In some embodiments, the TLR polypeptide may comprise certain amino acid mutations without effect on the association between a first TLR signaling domain and a second TLR signaling domain to form a TLR signaling moiety, thereby without effect on the induction of TLR signaling.

In some embodiments, the modified immune cell comprises a CAR system (e.g., a CAR fusion construct), wherein the CAR fusion construct comprises an anti-BCMA CAR molecule (e.g., a single anti-BCMA CAR or a tandem anti-BCMA CAR) and anti-BCMA TLR polypeptides. In some embodiments, each of the first polypeptide and the second polypeptide comprises from the N-terminus to the C-terminus: a signal peptide, a single anti-BCMA sdAb, a TLR4 transmembrane domain, and a TLR4 signaling domain (e.g., the cytoplasmic portion of TLR4). In some embodiments, the anti-BCMA CAR comprises from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-BCMA CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 67. In some embodiments, the anti-BCMA CAR comprises SEQ ID NO: 67. In some embodiments, the anti-BCMA CAR comprises from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-BCMA CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 66. In some embodiments, the anti-BCMA CAR comprises SEQ ID NO: 66. In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 62. In some embodiments the CAR fusion construct comprises SEQ ID NO: 62. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 63. In some embodiments the CAR fusion construct comprises SEQ ID NO: 63. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 64. In some embodiments the CAR fusion construct comprises SEQ ID NO: 64. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a tandem anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 61. In some embodiments the CAR fusion construct comprises SEQ ID NO: 61. In some embodiments, the TLR polypeptide coding sequence lacks the sequences of some or all of the putative upstream start codons. In some embodiments, the TLR polypeptide may comprise certain amino acid mutations without effect on the association between a first TLR signaling domain and a second TLR signaling domain to form a TLR signaling moiety, thereby without effect on the induction of TLR signaling. In some embodiments, the modified immune cell comprises a CAR system (e.g., a CAR fusion construct), wherein the CAR fusion construct comprises an anti-BCMA CAR molecule (e.g., a single anti-BCMA CAR or a tandem anti-BCMA CAR) and anti-GPRC5D TLR polypeptides. In some embodiments, each of the first polypeptide and the second polypeptide comprises from the N-terminus to the C-terminus: a signal peptide, an anti-GPRC5D scFv, a TLR4 transmembrane domain, and a TLR4 signaling domain (e.g., the cytoplasmic portion of TLR4). In some embodiments, the anti-BCMA CAR comprises from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-BCMA CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 66. In some embodiments, the anti-BCMA CAR comprises SEQ ID NO: 66. In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-GPRC5D TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 65. In some embodiments the CAR fusion construct comprises SEQ ID NO: 65. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 72. In some embodiments the CAR fusion construct comprises SEQ ID NO: 72. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 73. In some embodiments the CAR fusion construct comprises SEQ ID NO: 73. In some embodiments, the TLR polypeptide coding sequence lacks the sequences of some or all of the putative upstream start codons. In some embodiments, the TLR polypeptide may comprise certain amino acid mutations without effect on the association between a first TLR signaling domain and a second TLR signaling domain to form a TLR signaling moiety, thereby without effect on the induction of TLR signaling.

In some embodiments, the modified immune cell comprises a CAR system (e.g., a CAR fusion construct), wherein the CAR fusion construct comprises an anti-CLL1 CAR and anti-CD33 (e.g., anti-CD33 V domain and/or anti-CD33 C2 domain) TLR polypeptides. In some embodiments, the first polypeptide comprises from the N-terminus to the C-terminus: a signal peptide, an anti-CD33 V domain sdAb, a TLR2 transmembrane domain, and a TLR2 signaling domain (e.g., the cytoplasmic portion of TLR2), and the second polypeptides comprises from the N-terminus to the C-terminus: a signal peptide, an anti-CD33 C2 domain sdAb, a TLR1 transmembrane domain, and a TLR1 signaling domain (e.g., the cytoplasmic portion of TLR1). In some embodiments, the modified immune cell expresses an anti-CLL1 CAR. In some embodiments, the anti-CLL1 CAR comprises from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain. In some embodiments, the anti-CLL1 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 4. In some embodiments the anti-CLL1 CAR comprises SEQ ID NO: 4. In some embodiments, the modified immune cell expresses an anti-CLL1/CD33 dual dual CAR. In some embodiments, the anti-CLL1/CD33 dual CAR comprises from the N-terminus to the C-terminus: a signal peptide, an anti-CLL1 sdAb, an anti-CD33 V domain sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-CLL1/CD33 dual CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 43. In some embodiments the anti-CLL1/CD33 dual CAR comprises SEQ ID NO: 43. In some embodiments, the first polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 41. In some embodiments the first polypeptide comprises SEQ ID NO: 41. In some embodiments, the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 42. In some embodiments the second polypeptide comprises SEQ ID NO: 42. In some embodiments, the modified immune cell comprises a CAR system (e.g., a CAR fusion construct), wherein the CAR fusion construct comprises an anti-CLL1 CAR and anti-CD33 (e.g., anti-CD33 V domain and/or anti-CD33 C2 domain) TLR polypeptides. In some embodiments, the CAR fusion construct comprises, from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain, a P2A cleavage site, a signal peptide, an anti-CD33 V domain sdAb, a TLR2 transmembrane domain, a TLR2 signaling domain (e.g., the cytoplasmic portion of TLR2), a P2A cleavage site, a signal peptide, an anti-CD33 C2 domain sdAb, a TLR1 transmembrane domain, and a TLR1 signaling domain (e.g., the cytoplasmic portion of TLR1). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 3. In some embodiments, the TLR polypeptide coding sequence lacks the sequences of some or all of the putative upstream start codons. In some embodiments, the TLR polypeptide may comprise certain amino acid mutations without effect on the association between a first TLR signaling domain and a second TLR signaling domain to form a TLR signaling moiety, thereby without effect on the induction of TLR signaling.

Also within the scope of the present disclosure are variants of any of the TLR domains (e.g., a TLR transmembrane domain and/or a TLR signaling domain) described herein, such that the TLR domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such TLR domains comprising one or more amino acid variations may be referred to as variants. Mutation of amino acid residues of the TLR signaling domain may result in an increase in signaling transduction and enhanced stimulation of immune responses relative to co-stimulatory signaling domains that do not comprise the mutation. Mutation of amino acid residues of the TLR signaling domain may result in a decrease in signaling transduction and reduced stimulation of immune responses relative to co-stimulatory signaling domains that do not comprise the mutation.

In some embodiments, the TLR polypeptide comprises an amino acid sequence variant of the TLR domains (e.g., TLR transmembrane domain and/or TLR signaling domain) described herein. In some embodiments, the TLR polypeptide comprises an amino acid sequence variant of the TLR molecules (e.g., TLR transmembrane domain and/or TLR signaling domain of the TLR molecules) described herein. For example, it may be desirable to modulate the biological properties of the TLR polypeptide. Amino acid sequence variants of a TLR molecule thereof, such as the transmembrane domain and/or the signaling domain of a TLR molecule thereof, may be prepared by introducing appropriate modifications into the nucleotide sequence encoding the TLR molecule, or by peptide synthesis. Such modifications include, for example, deletions from, and/or insertions into and/or substitutions of residues within the amino acid sequences of the TLR molecule. Any combination of deletion, insertion, and substitution can be made to arrive at the final construct, provided that the final construct possesses the desired characteristics, e.g., TLR-binding and/or pro-inflammatory activities.

In some embodiments, the TLR molecule comprises one or more (e.g., at least 1, 2, 3, 4, 5, 10, 15, 20 amino acids or more) conservative substitutions compared to the sequence of any one of the TLR molecule described herein. In some embodiments, the TLR molecule comprises at least about 80% sequence identity, such as at least about any one of 850%, 87%, 90%, 91%, 92%, 93%, 94%, 95%, 96%, 97%, 98%, 99% or more sequence identity to the sequence of any one of the TLR molecules described herein. Like the TLR polypeptides described herein, the TLR polypeptide variants comprising modifying TLR molecules have similar anti-tumor activities and low toxicity.

Conservative substitutions are shown in Table A below.

TABLE A
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.
   Non-conservative substitutions will entail exchanging a member of one of these classes for another class.

One of skill in the art will recognize that any suitable method can be used for generating mutations in a gene of interest, including mutagenesis, polymerase chain reaction, homologous recombination, or any other genetic engineering technique known to a person of skill in the art. A mutation may involve a single nucleotide (such as a point mutation, which involves the removal, addition or substitution of a single nucleotide base within a DNA sequence) or it may involve the insertion or deletion of large numbers of nucleotides. Mutations can arise spontaneously as a result of events such as errors in the fidelity of DNA replication, or induced following exposure to chemical or physical mutagens. A mutation can also be site-directed through the use of particular targeting methods that are well known to persons of skill in the art.

A useful method for identification of residues or regions of a polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the polypeptide agent with its target (e.g., a first TLR signaling domain with a second TLR signaling domain) is affected. Further substitutions may be introduced at the amino acid locations demonstrating functional sensitivity to the initial substitutions. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues.

In some embodiments, a peptide tag (typically a short peptide sequence able to be recognized by available antisera or compounds) may be included for following expression and trafficking of the TLR polypeptide. A vast variety of tag peptides can be used in the TLR polypeptide described herein, without limitation, PK tag, FLAG octapeptide, MYC tag, HIS tag (usually a stretch of 4 to 10 histidine residues) and e-tag (U.S. Pat. No. 6,686,152). The tag peptide(s) may be independently positioned at the N-terminus of the protein, at its C-terminus, internally, or at any of these positions when several tags are employed. Tag peptides can be detected by immunodetection assays using anti-tag antibodies.

### Engineered Receptor

Any of the modified immune cells described above may further express an engineered receptor. Exemplary engineered receptor include, but are not limited to, CAR, engineered TCR, and TAC receptors. In some embodiments, the engineered receptor comprises an extracellular domain that specifically binds to an antigen (e.g., a tumor antigen), a transmembrane domain, and an intracellular signaling domain. In some embodiments, the intracellular signaling domain comprises a primary intracellular signaling domain and/or a co-stimulatory domain. In some embodiments, the intracellular signaling domain comprises an intracellular signaling domain of a TCR co-receptor. In some embodiments, the engineered receptor is encoded by a third nucleic acid operably linked to a promoter (such as a constitutive promoter or an inducible promoter). In some embodiments, the engineered receptor is introduced to the modified immune cell by inserting proteins into the cell membrane while passing cells through a microfluidic system, such as CELL SQUEEZE® (see, for example, U.S. Patent Application Publication No. 20140287509). The engineered receptor may enhance the function of the modified immune cell, such as by targeting the modified immune cell, by transducing signals, and/or by enhancing cytotoxicity of the modified immune cell. In some embodiments, the modified immune cell does not express an engineered receptor, such as CAR, TCR, or TAC receptor.

In some embodiments, the engineered receptor comprises one or more specific binding domains that target at least one tumor antigen, and one or more intracellular effector domains, such as one or more primary intracellular signaling domains and/or co-stimulatory domains.

In some embodiments, the engineered receptor is a chimeric antigen receptor (CAR). Many chimeric antigen receptors are known in the art and may be suitable for the modified immune cell of the present application. CARs can also be constructed with a specificity for any cell surface marker by utilizing antigen binding fragments or antibody variable domains of, for example, antibody molecules. Any method for producing a CAR may be used herein. See, for example, U.S. Pat. Nos. 6,410,319, 7,446,191, 7,514,537, 9,765,342B2, WO 2002/077029, WO2015/142675, US2010/065818, US 2010/025177, US 2007/059298, WO2017025038A1, and Berger C. et al., J. Clinical Investigation 118: 1 294-308 (2008), which are hereby incorporated by reference. In some embodiments, the modified immune cell is a CAR-T cell.

CARs of the present application comprise an extracellular domain comprising at least one targeting domain that specifically binds at least one tumor antigen, a transmembrane domain, and an intracellular signaling domain. In some embodiments, the intracellular signaling domain generates a signal that promotes an immune effector function of the CAR-containing cell, e.g., a CAR-T cell. “Immune effector function or immune effector response” refers to function or response, e.g., of an immune effector cell, that enhances or promotes an immune attack of a target cell. For example, an immune effector function or response may refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). In some embodiments, the CAR has an intracellular signaling domain with an attenuated immune effector function. In some embodiments, the CAR has an intracellular signaling domain having no more than about any of 90%, 80%, 70%, 60%, 50%, 40%, 30%, 20%, 10% or less of an immune effector function (such as cytolytic function against target cells) compared to a CAR having a full-length and wildtype CD3ζ and optionally one or more co-stimulatory domains. In some embodiments, the intracellular signaling domain generates a signal that promotes proliferation and/or survival of the CAR containing cell. In some embodiments, the CAR comprises one or more intracellular signaling domains selected from the signaling domains of CD28, CD137, CD3, CD27, CD40, ICOS, GITR, and OX40. The signaling domain of a naturally occurring molecule can comprise the entire intracellular (i.e., cytoplasmic) portion, or the entire native intracellular signaling domain, of the molecule, or a fragment or derivative thereof.

In some embodiments, the intracellular signaling domain of a CAR comprises a primary intracellular signaling domain. “Primary intracellular signaling domain” refers to cytoplasmic signaling sequence that acts in a stimulatory manner to induce immune effector functions. In some embodiments, the primary intracellular signaling domain contains a signaling motif known as Immunoreceptor Tyrosine-based Activation Motif, or ITAM. In some embodiments, the primary intracellular signaling domain comprises a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP 12. In some embodiments, the primary intracellular signaling domain comprises a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP 12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). In some embodiments, the CAR comprises a nonfunctional or attenuated CD3 zeta (i.e., CD3ζ or CD3z) signaling domain. In some embodiments, the intracellular signaling domain does not comprise a primary intracellular signaling domain. An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wildtype primary intracellular signaling domain.

In some embodiments, the intracellular signaling domain of a CAR comprises one or more (such as any of 1, 2, 3, or more) co-stimulatory domains. “Co-stimulatory domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD1la/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD1 Ib, ITGAX, CD1 Ic, ITGB1, CD29, ITGB2, CD18, LFA-1, ITGB7, NKG2D, NKG2C, TGFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, the CAR comprises a single co-stimulatory domain. In some embodiments, the CAR comprises two or more co-stimulatory domains. In some embodiments, the intracellular signaling domain comprises a functional primary intracellular signaling domain and one or more co-stimulatory domains. In some embodiments, the CAR does not comprise a functional primary intracellular signaling domain (such as CD3ζ). In some embodiments, the CAR comprises an intracellular signaling domain consisting of or consisting essentially of one or more co-stimulatory domains. In some embodiments, the CAR comprises an intracellular signaling domain consisting of or consisting essentially of a nonfunctional or attenuated primary intracellular signaling domain (such as a mutant CD3ζ) and one or more co-stimulatory domains. Upon binding of the targeting domain to tumor antigen, the co-stimulatory domains of the CAR may transduce signals for enhanced proliferation, survival and differentiation of the engineered immune cells having the CAR (such as T cells), and inhibit activation induced cell death. In some embodiments, the one or more co-stimulatory signaling domains are derived from one or more molecules selected from the group consisting of CD27, CD28, 4-1BB (i.e., CD137), OX40, CD30, CD40, CD3, lymphocyte function-associated antigen-1 (LFA-1), CD2, CD7, LIGHT, NKG2C, B7-H3 and ligands that specially bind to CD83.

In some embodiments, the intracellular signaling domain of the CAR comprises a co-stimulatory signaling domain derived from CD28. In some embodiments, the intracellular signaling domain comprises a cytoplasmic signaling domain of CD3ζ and a co-stimulatory signaling domain of CD28. In some embodiments, the intracellular signaling domain in the chimeric receptor of the present application comprises a co-stimulatory signaling domain derived from 4-1BB (i.e., CD137). In some embodiments, the intracellular signaling domain comprises a cytoplasmic signaling domain of CD3ζ and a co-stimulatory signaling domain of 4-1BB.

In some embodiments, the intracellular signaling domain of the CAR comprises a co-stimulatory signaling domain of CD28 and a co-stimulatory signaling domain of 4-1BB. In some embodiments, the intracellular signaling domain comprises a cytoplasmic signaling domain of CD3ζ, a co-stimulatory signaling domain of CD28, and a co-stimulatory signaling domain of 4-1BB. In some embodiments, the intracellular signaling domain comprises a polypeptide comprising from the N-terminus to the C-terminus: a co-stimulatory signaling domain of CD28, a co-stimulatory signaling domain of 4-1BB, and a cytoplasmic signaling domain of CD3ζ.

In some embodiments, the targeting domain of the CAR is an antibody or an antibody fragment, such as a scFv, a Fv, a Fab, a (Fab′)₂, a single domain antibody (sdAb), or a V_{H}H domain. In some embodiments, the targeting domain of the CAR is a ligand or an extracellular portion of a receptor that specifically binds to a tumor antigen. In some embodiments, the one or more targeting domains of the CAR specifically bind to a single tumor antigen. In some embodiments, the CAR is a bispecific or multispecific CAR with targeting domains that bind two or more tumor antigens. In some embodiments, the tumor antigen is selected from the group consisting of CD19, NKG2D, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance, and combinations thereof. In some embodiments, the CAR specifically binds to a target antigen selected from the group consisting of BCMA, NY-ESO-1, VEGFR2, MAGE-A3, AFP, CD4, CD19, CD20, CD22, CD30, CD33, CD38, CD70, CD123, CEA, EGFR (such as EGFRvIII), GD2, GPC-2, GPC3, CLDN18.2, HER2, LILRB4, IL-13Rα2, IGF1R, mesothelin, PSMA, ROR1, WT1, NKG2D, CLL1, TGFaRII, TGFbRII, CCR5, CXCR4, CCR4, HPV related antigens, and EBV related antigens (e.g., LMP1 or LMP2).

In some embodiments, the CAR is an anti-CD19 CAR. A wide variety of antigen binding domain sequences can be used as the targeting domains of the CAR. See, e.g., WO2012/079000, which is incorporated herein in its entirety. In some embodiments, the anti-CD19 CAR comprises from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-CD19 scFv comprises the amino acid sequence of SEQ ID NO: 6. In some embodiments, the anti-CD19 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 2. In some embodiments, the anti-CD19 CAR comprises SEQ ID NO: 2.

In some embodiments, the anti-CD19 CAR is part of a CAR system (e.g., a CAR fusion construct), wherein the CAR fusion construct comprises the anti-CD19 CAR molecule and anti-CD20 TLR polypeptides. In some embodiments, each of a first polypeptide and a second polypeptide comprises from the N-terminus to the C-terminus: a signal peptide, an anti-CD20 scFv, a TLR4 transmembrane domain, and a TLR4 signaling domain (e.g., the cytoplasmic portion of TLR4). In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD20 scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1. In some embodiments the CAR fusion construct comprises SEQ ID NO: 1.

In some embodiments, the anti-CD19 CAR is part of a CAR system (e.g., a CAR fusion construct), wherein the CAR fusion construct comprises the anti-CD19 CAR molecule and anti-NKG2D TLR polypeptides. In some embodiments, each of a first polypeptide and a second polypeptide comprises from the N-terminus to the C-terminus: a signal peptide, an anti-NKG2D ECD, a TLR4 transmembrane domain, and a TLR4 signaling domain (e.g., the cytoplasmic portion of TLR4). In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 59. In some embodiments the CAR fusion construct comprises SEQ ID NO: 59. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 60. In some embodiments the CAR fusion construct comprises SEQ ID NO: 60.

In some embodiments, the CAR is an anti-CLL1 CAR. A wide variety of antigen binding domain sequences can be used as the targeting domains of the CAR. See, e.g., WO2012/079000, which is incorporated herein in its entirety. In some embodiments, the anti-CLL1 CAR comprises from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain. In some embodiments, the anti-CLL1 sdAb comprises the amino acid sequence of SEQ ID NO: 14. In some embodiments, the anti-CLL1 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 4. In some embodiments the anti-CLL1 CAR comprises SEQ ID NO: 4.

In some embodiments, the CAR is an anti-CLL1/CD33 dual CAR. A wide variety of antigen binding domain sequences can be used as the targeting domains of the CAR. See, e.g., WO2012/079000, which is incorporated herein in its entirety. In some embodiments, the anti-CLL1/CD33 dual CAR comprises from the N-terminus to the C-terminus: a signal peptide, an anti-CLL1 sdAb, an anti-CD33 V domain sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-CLL1 sdAb comprises the amino acid sequence of SEQ ID NO: 14. In some embodiments, the anti-CD33 V domain sdAb comprises the amino acid sequence of SEQ ID NO: 21. In some embodiments, the anti-CLL1/CD33 dual CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 43. In some embodiments the anti-CLL1/CD33 dual CAR comprises SEQ ID NO: 43.

In some embodiments, the anti-CLL1 CAR is part of a CAR system (e.g., a CAR fusion construct), wherein the CAR fusion construct comprises an anti-CLL1 CAR and anti-CD33 TLR polypeptides. In some embodiments, each of a first polypeptide and a second polypeptide comprises from the N-terminus to the C-terminus: a signal peptide, an anti-CD33 sdAb, a TLR4 transmembrane domain, and a TLR4 signaling domain (e.g., the cytoplasmic portion of TLR4). In some embodiments, each of the first polypeptide and the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 40. In some embodiments, each of the first polypeptide and the second polypeptide comprises SEQ ID NO: 40. In some embodiments, the CAR fusion construct comprises, from the N-terminus to the C-terminus: a signal peptide, an anti-CLL1 sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD33 sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 71. In some embodiments the CAR fusion construct comprises SEQ ID NO: 71.

In some embodiments, the anti-CLL1 CAR is part of a CAR system (e.g., a CAR fusion construct), wherein the CAR fusion construct comprises an anti-CLL1 CAR and anti-CD33 (e.g., anti-CD33 V domain and/or anti-CD33 C2 domain) TLR polypeptides. In some embodiments, a first polypeptide comprises from the N-terminus to the C-terminus: a signal peptide, an anti-CD33 V domain sdAb, a TLR2 transmembrane domain, and a TLR2 signaling domain (e.g., the cytoplasmic portion of TLR2), and a second polypeptides comprises from the N-terminus to the C-terminus: a signal peptide, an anti-CD33 C2 domain sdAb, a TLR1 transmembrane domain, and a TLR1 signaling domain (e.g., the cytoplasmic portion of TLR1). In some embodiments, the first polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 41. In some embodiments the first polypeptide comprises SEQ ID NO: 41. In some embodiments, the second polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 42. In some embodiments the second polypeptide comprises SEQ ID NO: 42. In some embodiments, the CAR fusion construct comprises, from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain, a P2A cleavage site, a signal peptide, an anti-CD33 V domain sdAb, a TLR2 transmembrane domain, a TLR2 signaling domain (e.g., the cytoplasmic portion of TLR2), a P2A cleavage site, a signal peptide, an anti-CD33 C2 domain sdAb, a TLR1 transmembrane domain, and a TLR1 signaling domain (e.g., the cytoplasmic portion of TLR1). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 3.

In some embodiments, the CAR is an anti-GPC3 CAR. A wide variety of antigen binding domain sequences can be used as the targeting domains of the CAR. See, e.g., WO2012/079000, which is incorporated herein in its entirety. In some embodiments, the anti-GPC3 CAR comprises from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the anti-GPC3 scFv comprises the amino acid sequence of SEQ ID NO: 55. In some embodiments, the anti-GPC3 CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 56. In some embodiments the anti-GPC3 CAR comprises SEQ ID NO: 56.

In some embodiments, the anti-GPC3 CAR is part of a CAR system (e.g., a CAR fusion construct), wherein the CAR fusion construct comprises an anti-GPC3 CAR and anti-NKG2D TLR polypeptides. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 57. In some embodiments the CAR fusion construct comprises SEQ ID NO: 57. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 58. In some embodiments the CAR fusion construct comprises SEQ ID NO: 58.

In some embodiments, the CAR is an anti-BCMA CAR (e.g., a single anti-BCMA CAR or a tandem anti-BCMA CAR). In some embodiments, the anti-BCMA CAR is a single anti-BCMA CAR. In some embodiments, the anti-BCMA CAR is a tandem anti-BCMA CAR. A wide variety of antigen binding domain sequences can be used as the targeting domains of the CAR. See, e.g., WO2012/079000, which is incorporated herein in its entirety. In some embodiments, the single anti-BCMA CAR comprises from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the single anti-BCMA sdAb comprises the amino acid sequence of SEQ ID NO: 68. In some embodiments, the single anti-BCMA CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 67. In some embodiments the single anti-BCMA CAR comprises SEQ ID NO: 67. In some embodiments, the tandem anti-BCMA CAR comprises from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain. In some embodiments, the tandem anti-BCMA sdAb comprises the amino acid sequence of SEQ ID NO: 44. In some embodiments, the tandem anti-BCMA CAR comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 66. In some embodiments the tandem anti-BCMA CAR comprises SEQ ID NO: 66.

In some embodiments, the anti-BCMA CAR is part of a CAR system (e.g., a CAR fusion construct), wherein the CAR fusion construct comprises an anti-BCMA CAR (e.g., a single anti-BCMA CAR or a tandem anti-BCMA CAR) and anti-BCMA TLR polypeptides. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 62. In some embodiments the CAR fusion construct comprises SEQ ID NO: 62. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 63. In some embodiments the CAR fusion construct comprises SEQ ID NO: 63. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 64. In some embodiments the CAR fusion construct comprises SEQ ID NO: 64. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a tandem anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 61. In some embodiments the CAR fusion construct comprises SEQ ID NO: 61.

In some embodiment embodiments, the anti-BCMA CAR is part of a CAR system (e.g., a CAR fusion construct), wherein the CAR fusion construct comprises an anti-BCMA CAR (e.g., a single anti-BCMA CAR or a tandem anti-BCMA CAR) and anti-GPRC5D TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 65. In some embodiments the CAR fusion construct comprises SEQ ID NO: 65. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 72. In some embodiments the CAR fusion construct comprises SEQ ID NO: 72. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 73. In some embodiments the CAR fusion construct comprises SEQ ID NO: 73.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRFl), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD1 Ib, ITGAX, CD1 Ic, ITGB1, CD29, ITGB2, CD18, LFA-1, ITGB7, TGFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of the CAR is a CD4, CD3, CD8α, or CD28 transmembrane domain. In some embodiments, the transmembrane domain of the CAR comprises a transmembrane domain of CD8α.

In some embodiments, the extracellular domain is connected to the transmembrane domain by a hinge region. In one embodiment, the hinge region comprises the hinge region of CD8α.

In some embodiments, the CAR comprises a signal peptide, such as a CD8αSP.

In some embodiments, the engineered receptor is a modified T-cell receptor. In some embodiments, the engineered TCR is specific for a tumor antigen. In some embodiments, the tumor antigen is selected from the group consisting of CD19, CLL1, GPC3, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). In some embodiments, the modified immune cell is a TCR-T cell.

The TCR receptor complex is an octomeric complex formed by variable TCR receptor α and β chains (γ and δ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane domain of each subunit form a polar network of interactions that hold the complex together. TCR complex has the function of activating signaling cascades in T cells.

In some embodiments, the engineered receptor is an engineered TCR comprising one or more T-cell receptor (TCR) fusion proteins (TFPs). Exemplary TFPs have been described, for example, in US20170166622A1, which is incorporated herein by reference. In some embodiments, the TFP comprises an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the TFP comprising a TCR subunit comprising at least a portion of a TCR extracellular domain, and a TCR intracellular domain comprising a stimulatory domain from an intracellular signaling domain of CD3 epsilon; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 gamma; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 delta; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR alpha; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR beta; and an antigen binding domain wherein the TCR subunit and the antigen binding domain are operatively linked, and wherein the TFP incorporates into a TCR when expressed in a T cell.

In some embodiments, the engineered receptor is a T-cell antigen coupler (TAC) receptor. Exemplary TAC receptors have been described, for example, in US20160368964A1, which is incorporated herein by reference. In some embodiments, the TAC comprises a targeting domain, a TCR-binding domain that specifically binds a protein associated with the TCR complex, and a T-cell receptor signaling domain. In some embodiments, the targeting domain is an antibody fragment, such as scFv or V_{H}H, which specifically binds to a tumor antigen. In some embodiments, the targeting domain is a designed Ankyrin repeat (DARPin) polypeptide. In some embodiments, the tumor antigen is selected from the group consisting of CD19, GPC3, CLL1, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance. In some embodiments, the protein associated with the TCR complex is CD3, such as CD3ε. In some embodiments, the TCR-binding domain is a single chain antibody, such as scFv, or a V_{H}H. In some embodiments, the TCR-binding domain is derived from UCHT1. In some embodiments, the TAC receptor comprises a cytosolic domain and a transmembrane domain. In some embodiments, the T-cell receptor signaling domain comprises a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD154. In some embodiments, the TAC receptor comprises a transmembrane domain and a cytosolic domain derived from CD4. In some embodiments, the TAC receptor comprises a transmembrane domain and a cytosolic domain derived from CD8 (such as CD8α).

T cell co-receptors are expressed as membrane protein on T cells. They can provide stabilization of the TCR: peptide: MHC complex and facilitate signal transduction. The two subtypes of T cell co-receptor, CD4 and CD8, display strong specificity for particular MHC classes. The CD4 co-receptor can only stabilize TCR: MHC II complexes while the CD8 co-receptor can only stabilize the TCR: MHC I complex. The differential expression of CD4 and CD8 on different T cell types results in distinct T cell functional subpopulations. CD8+ T cells are cytotoxic T cells.

CD4 is a glycoprotein expressed on the surface of immune cells such as T helper cells, monocytes, macrophages, and dendritic cells. CD4 has four immunoglobulin domains (Di to D4) exposed on the extracellular cell surface. CD4 contains a special sequence of amino acids on its short cytoplasmic/intracellular tail, which allow CD4 tail to recruit and interact with the tyrosine kinase Lck. When the TCR complex and CD4 each bind to distinct regions of the MHC II molecule, the close proximity between the TCR complex and CD4 allows Lck bound to the cytoplasmic tail of CD4 to tyrosine-phosphorylate the Immunoreceptor Tyrosine Activation Motifs (ITAM) on the cytoplasmic domains of CD3, thus amplifying TCR generated signal.

CD8 is a glycoprotein of either a homodimer composed of two a chains (less common), or a heterodimer composed of one α and one β chain (more common), each comprising an immunoglobulin variable (IgV)-like extracellular domain connected to the membrane by a thin stalk, and an intracellular tail. CD8 is predominantly expressed on the surface of cytotoxic T cells, but can also be found on natural killer cells, cortical thymocytes, and dendritic cells. The CD8 cytoplasmic tail interacts with Lck, which phosphorylates the cytoplasmic CD3 and ζ-chains of the TCR complex once TCR binds its specific antigen. Tyrosine-phosphorylation on the cytoplasmic CD3 and ζ-chains initiates a cascade of phosphorylation, eventually leading to gene transcription.

In some embodiments, the modified immune cell expresses more than one engineered receptors, such as any combination of CAR, TCR, TAC receptor.

In some embodiments, the engineered receptor (such as CAR, TCR, or TAC) expressed by the modified immune cell targets one or more tumor antigens. Tumor antigens are proteins that are produced by tumor cells that can elicit an immune response, particularly T-cell mediated immune responses. The selection of the targeted antigen of the disclosure will depend on the particular type of cancer to be treated. Exemplary tumor antigens include, for example, a glioma-associated antigen, carcinoembryonic antigen (CEA), 3-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, CLL1, BCMA, GPC3, CD19, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### Nucleic Acids

The modified immune cells described herein comprises one or more heterologous nucleic acids sequence(s) encoding any one of the TLR polypeptides (e.g., a first polypeptide and/or a second polypeptide) and/or engineered receptors described herein.

In some embodiments, there is provided an isolated nucleic acid comprising a nucleic acid sequence encoding any one of the polypeptides (e.g., TLR polypeptides) described herein. In some embodiments, there is provided an isolated nucleic acid comprising a nucleic acid sequence encoding any one of the engineered receptors described herein. In some embodiments, the nucleic acid is a DNA. In some embodiments, the nucleic acid is a RNA. In some embodiments, the nucleic acid is linear. In some embodiments, the nucleic acid is circular.

The nucleic acid sequence encoding a first polypeptide, a second polypeptide, and/or the nucleic acid encoding the engineered receptor may be operably linked to one or more regulatory sequences. Exemplary regulatory sequences that control the transcription and/or translation of a coding sequence are known in the art and may include, but not limited to, a promoter, additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), translation (e.g. an initiator Met, tripartite leader sequences, IRES ribosome binding sites, signal peptides, etc.), and insertion site for introducing an insert into the viral vector. In some embodiments, the regulatory sequence is a promoter, a transcriptional enhancer and/or a sequence that allows for proper expression of the TLR polypeptide and/or the engineered receptor.

The term “regulatory sequence” or “control sequence” refers to a DNA sequence that affects the expression of a coding sequence to which it is operably linked. The nature of such regulatory sequences differs depending upon the host organism. In prokaryotes, regulatory sequences generally include promoters, ribosomal binding sites, and terminators. In eukaryotes, regulatory sequences include promoters, terminators and, in some instances, enhancers, transactivators or transcription factors.

The term “operably linked” refers to a juxtaposition wherein the components so described are in a relationship permitting them to function in their intended manner. A regulatory sequence “operably linked” to a coding sequence is ligated in such a way that expression of the coding sequence is achieved under conditions compatible with the regulatory sequences.

As used herein, a “promoter” or a “promoter region” refers to a segment of DNA or RNA that controls transcription of the DNA or RNA to which it is operatively linked. The promoter region includes specific sequences that are involved in RNA polymerase recognition, binding and transcription initiation. In addition, the promoter includes sequences that modulate recognition, binding and transcription initiation activity of RNA polymerase (i.e., binding of one or more transcription factors). These sequences can be cis acting or can be responsive to trans acting factors. Promoters, depending upon the nature of the regulation, can be constitutive or regulated. Regulated promoters can be inducible or environmentally responsive (e.g. respond to cues such as pH, anaerobic conditions, osmoticum, temperature, light, or cell density). Many such promoter sequences are known in the art. See, for example, U.S. Pat. Nos. 4,980,285; 5,631,150; 5,707,928; 5,759,828; 5,888,783; 5,919,670, and, Sambrook, et al., Molecular Cloning: A Laboratory Manual, 2nd Ed., Cold Spring Harbor Press (1989).

In some embodiments, the nucleic acid sequence encoding the first polypeptide is operably linked to a first promoter. In some embodiments, the nucleic acid sequence encoding the second polypeptide is operably linked to a second promoter. In some embodiments, the first polypeptide is the same as the second polypeptide, and the first nucleic acid encodes both the first polypeptide and the second polypeptide. In some embodiments, the nucleic acid sequence encoding the first polypeptide and the nucleic acid sequence encoding the second polypeptide are operably linked to the same promoter. In some embodiments, the nucleic acid sequence encoding the first polypeptide and the nucleic acid sequence encoding the second polypeptide are operably linked to separate promoters.

In some embodiments, the modified immune cell comprises a third nucleic acid encoding the engineered receptor. In some embodiments, the first nucleic acid and the third nucleic acid are operably linked to the same promoter. In some embodiments, the first nucleic acid and the third nucleic acid are operably linked to separate promoters. In some embodiments, the second nucleic acid and the third nucleic acid are operably linked to the same promoter. In some embodiments, the second nucleic acid and the third nucleic acid are operably linked to separate promoters. In some embodiments, the first nucleic acid, the second nucleic acid, and the third nucleic acid are operably linked to the same promoter. In some embodiments, the first nucleic acid, the second nucleic acid, and the third nucleic acid are operably linked to separate promoters.

In some embodiments, the promoter is an endogenous promoter. For example, a nucleic acid encoding the first polypeptide, the second polypeptide, and/or the engineered receptor may be knocked-in to the genome of the modified immune cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the modified immune cell. In some embodiments, wherein the modified immune cell is a T cell, the promoter is a T cell activation-dependent promoter (such as an IL-2 promoter, an NFAT promoter, or an NFκB promoter). In some embodiments, the promoter is a heterologous promoter.

Varieties of promoters have been explored for gene expression in mammalian cells, and any of the promoters known in the art may be used in the present application. Promoters may be roughly categorized as constitutive promoters or regulated promoters, such as inducible promoters. In some embodiments, the heterologous nucleic acid sequence encoding the first polypeptide, the second polypeptide, and/or the engineered receptor is operably linked to a constitutive promoter. In some embodiments, the heterologous nucleic acid sequence encoding the first polypeptide, the second polypeptide, and/or the engineered receptor is operably linked to an inducible promoter. In some embodiments, a first constitutive promoter is operably linked to the nucleic acid sequence encoding the first polypeptide, a second constitutive promoter is operably linked to the nucleic acid sequence encoding the second polypeptide, and an inducible promoter is operably linked to the nucleic acid sequence encoding the engineered receptor. In some embodiments, a first constitutive promoter is operably linked to the nucleic acid sequence encoding the first polypeptide, a second constitutive promoter is operably linked to the nucleic acid sequence encoding the second polypeptide, and third constitutive promoter is operably linked to the nucleic acid sequence encoding the engineered receptor. In some embodiments, a first constitutive promoter is operably linked to the nucleic acid sequence encoding the first polypeptide, a second constitutive promoter is operably linked to the nucleic acid sequence encoding the second polypeptide, and an inducible promoter is operably linked to the nucleic acid sequence encoding the engineered receptor. In some embodiments, a first constitutive promoter is operably linked to the nucleic acid sequence encoding the first polypeptide, a second constitutive promoter is operably linked to the nucleic acid sequence encoding the engineered receptor, and an inducible promoter is operably linked to the nucleic acid sequence encoding the second polypeptide. In some embodiments, a first constitutive promoter is operably linked to the nucleic acid sequence encoding the second polypeptide, a second constitutive promoter is operably linked to the nucleic acid sequence encoding the engineered receptor, and an inducible promoter is operably linked to the nucleic acid sequence encoding the first polypeptide. In some embodiments, a first constitutive promoter is operably linked to the nucleic acid sequence encoding the first polypeptide, a first inducible promoter is operably linked to the nucleic acid sequence encoding the engineered receptor, and a second inducible promoter is operably linked to the nucleic acid sequence encoding the second polypeptide. In some embodiments, a first constitutive promoter is operably linked to the nucleic acid sequence encoding the second polypeptide, a first inducible promoter is operably linked to the nucleic acid sequence encoding the engineered receptor, and a second inducible promoter is operably linked to the nucleic acid sequence encoding the first polypeptide. In some embodiments, a first constitutive promoter is operably linked to the nucleic acid sequence encoding the engineered receptor, a first inducible promoter is operably linked to the nucleic acid sequence encoding the second polypeptide, and a second inducible promoter is operably linked to the nucleic acid sequence encoding the first polypeptide.

In some embodiments, a first inducible promoter is operably linked to the nucleic acid sequence encoding the first polypeptide, a second inducible promoter is operably linked to the nucleic acid sequence encoding the second polypeptide, and a third inducible promoter is operably linked to the nucleic acid sequence encoding the engineered receptor. In some embodiments, a first inducible promoter is operably linked to the nucleic acid sequence encoding the first polypeptide, a second inducible promoter is operably linked to the nucleic acid sequence encoding the second polypeptide, and a constitutive promoter is operably linked to the nucleic acid sequence encoding the engineered receptor. In some embodiments, a first inducible promoter is operably linked to the nucleic acid sequence encoding the first polypeptide, a second inducible promoter is operably linked to the nucleic acid sequence encoding the engineered receptor, and a constitutive promoter is operably linked to the nucleic acid sequence encoding the second polypeptide. In some embodiments, a first inducible promoter is operably linked to the nucleic acid sequence encoding the second polypeptide, a second inducible promoter is operably linked to the nucleic acid sequence encoding the engineered receptor, and a constitutive promoter is operably linked to the nucleic acid sequence encoding the first polypeptide. In some embodiments, a first inducible promoter is operably linked to the nucleic acid sequence encoding the first polypeptide, a first constitutive promoter is operably linked to the nucleic acid sequence encoding the engineered receptor, and a second constitutive promoter is operably linked to the nucleic acid sequence encoding the second polypeptide. In some embodiments, a first inducible promoter is operably linked to the nucleic acid sequence encoding the second polypeptide, a first constitutive promoter is operably linked to the nucleic acid sequence encoding the engineered receptor, and a second constitutive promoter is operably linked to the nucleic acid sequence encoding the first polypeptide. In some embodiments, a first inducible promoter is operably linked to the nucleic acid sequence encoding the engineered receptor, a first constitutive promoter is operably linked to the nucleic acid sequence encoding the second polypeptide, and a second constitutive promoter is operably linked to the nucleic acid sequence encoding the first polypeptide.

In some embodiments, the first inducible promoter is inducible by a first inducing condition, the second inducible promoter is inducible by a second inducing condition, and the third inducible promoter is inducible by a third inducing condition. In some embodiments, the first inducing condition is the same as the second inducing condition. In some embodiments, the first inducing condition is the same as the third inducing condition. In some embodiments, the second inducing condition is the same as the third inducing condition. In some embodiments, the first inducing condition, the second inducing condition, and the third inducing condition are all the same. In some embodiments, the first inducible promoter and the second inducible promoter are induced simultaneously. In some embodiments, the first inducible promoter and the third inducible promoter are induced simultaneously. In some embodiments, the second inducible promoter and the third inducible promoter are induced simultaneously. In some embodiments, the first inducible promoter, the second inducible promoter, and the third inducible promoter are all induced simultaneously. In some embodiments, the first inducible promoter, the second inducible promoter, and the third inducible promoter are induced sequentially, for example, the first inducible promoter is induced prior to the second inducible promoter and the second inducible promoter is induced prior to the third inducible promoter, the first inducible promoter is induced after the second inducible promoter and the second inducible promoter is induced prior to the third inducible promoter, or the first inducible promoter is induced after the second inducible promoter and the second inducible promoter is induced after to the third inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. In some embodiments, the promoter is a hEF1α promoter.

In some embodiments, the promoter is an inducible promoter. Inducible promoters belong to the category of regulated promoters. The inducible promoter can be induced by one or more conditions, such as a physical condition, microenvironment of the modified immune cell, or the physiological state of the modified immune cell, an inducer (i.e., an inducing agent), or a combination thereof. In some embodiments, the inducing condition does not induce the expression of endogenous genes in the modified immune cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the modified immune cell.

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most established system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. In some embodiments, the polypeptide is expressed by the modified immune cell. In some embodiments, the polypeptide is encoded by a nucleic acid in the heterologous nucleic acid. Many polypeptide inducers are also known in the art, and they may be suitable for use in the present application. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present application. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is a light-inducible promoter, and the inducing condition is light. Light inducible promoters for regulating gene expression in mammalian cells are also well-known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly divided into two categories based on their regulations of (1) DNA binding or (2) recruitment of a transcriptional activation domain to a DNA bound protein. For instance, synthetic mammalian blue light controlled transcription system based on melanopsin which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present application.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and AP1. The immediate early genes comprise radiation responsive sequences in their promoter regions. Consensus sequences CC(A/T)₆GG have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. And tumor growth was delayed significantly in the IL12 transgene plus heat treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present application.

In some embodiments, the promoter is inducible by a redox state. Exemplary promoters that are inducible by redox state include inducible promoter and hypoxia inducible promoters. For instance, Post D E et al developed hypoxia-inducible factor (HIF) responsive promoter which specifically and strongly induce transgene expression in HIF-active tumor cells (Gene Ther. 8: 1801-1807 (2001); Cancer Res. 67: 6872-6881 (2007)).

In some embodiments, the promoter is inducible by the physiological state, such as an endogenous activation signal, of the modified immune cell. In some embodiments, wherein the modified immune cell is a T cell, the promoter is a T cell activation-dependent promoter, which is inducible by the endogenous activation signal of the modified T cell. In some embodiments, the modified T cell is activated by an inducer, such as phorbol myristate acetate (PMA), ionomycin, or phytohaemagglutinin. In some embodiments, the modified T cell is activated by recognition of a tumor antigen on the tumor cells via the engineered receptor (such as CAR, TCR or TAC). In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

The heterologous nucleic acid sequences(s) described herein can be present in a heterologous gene expression cassette, which comprises one or more protein-coding sequences and optionally one or more promoters. In some embodiments, the heterologous gene expression cassette comprises a single protein-coding sequence. In some embodiments, the heterologous gene expression cassette comprises two or more protein-coding sequences driven by a single promoter (i.e., polycistronic). In some embodiments, the heterologous gene expression cassette further comprises one or more regulatory sequences (such as 5′UTR, 3′UTR, enhancer sequence, IRES, transcription termination sequence), recombination sites, one or more selection markers (such as antibiotic resistance gene, reporter gene, etc.), signal sequence, or combinations thereof.

In some embodiments, there is provided a vector comprising any one of the nucleic acids encoding the first polypeptides and/or the engineered receptors described herein. In some embodiments, there is provided a vector comprising a first nucleic acid sequence encoding any one of the first polypeptides described herein and a second nucleic acid sequence encoding any one of the engineered receptors described herein. In some embodiments, the first nucleic acid sequence encoding the first polypeptide is fused to the second nucleic acid sequence encoding the engineered receptor via a third nucleic acid sequence encoding a self-cleavable linker, such as P2A, T2A, E2A, or F2A peptide. In some embodiments, the P2A sequence is GSGATNFSLLKQAGDVEENPGP (SEQ ID NO: 24). In some embodiments, there is provided a composition comprising a first vector comprising a first nucleic acid sequence encoding any one of the first polypeptides described herein, and a second vector comprising a second nucleic acid sequence encoding any one of the engineered receptors described herein. In some embodiments, there is provided a vector comprising a first nucleic acid sequence encoding a CAR (e.g., a CD19 CAR or CLL1 CAR) and a second nucleic acid sequence encoding a first polypeptide, wherein the first nucleic acid sequence is fused to the second nucleic acid sequence via a third nucleic acid sequence encoding a self-cleavable linker, such as P2A. In some embodiments, the vector comprises a nucleic acid sequence encoding the amino acid sequence of SEQ ID NO: 1.

In some embodiments, there is provided a vector comprising any one of the nucleic acids encoding the first polypeptides, the second polypeptides, and/or the engineered receptors described herein. In some embodiments, there is provided a vector comprising a first nucleic acid sequence encoding any one of the first polypeptides described herein, a second nucleic acid sequence encoding any one of the second polypeptides described herein, and a third nucleic acid sequence encoding any one of the engineered receptors described herein. In some embodiments, the first nucleic acid sequence encoding the first polypeptide is fused to the second nucleic acid sequence encoding the engineered receptor via a fourth nucleic acid sequence encoding a self-cleavable linker, such as P2A, T2A, E2A, or F2A peptide. In some embodiments, the first nucleic acid sequence encoding the first polypeptide is additionally fused to the third nucleic acid sequence encoding the second polypeptide via a fifth nucleic acid sequence encoding a self-cleavable linker, such as P2A, T2A, E2A, or F2A peptide. In some embodiments, the P2A sequence is GSGATNFSLLKQAGDVEENPGP (SEQ ID NO: 24). In some embodiments, there is provided a composition comprising a first vector comprising a first nucleic acid sequence encoding any one of the first polypeptides described herein, a second vector comprising a second nucleic acid sequence encoding any one of the second polypeptides described herein, and a third vector comprising a third nucleic acid sequence encoding any one of the engineered receptors described herein. In some embodiments, there is provided a vector comprising a first nucleic acid sequence encoding a CAR (e.g., a CD19 CAR or a CLL1 CAR), a second nucleic acid sequence encoding a first polypeptide, and a third nucleic acid sequence encoding a second polypeptide, wherein the first nucleic acid sequence is fused to the second nucleic acid sequence via a fourth nucleic acid sequence encoding a self-cleavable linker, such as P2A, and wherein the second nucleic acid sequence is fused to the third nucleic acid sequence via a fifth nucleic acid sequence encoding a self-cleavable linker, such as P2A. In some embodiments, the vector comprises a nucleic acid sequence encoding the amino acid sequence of SEQ ID NO: 3.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In general, a suitable vector contains an origin of replication functional in at least one organism, a promoter sequence, convenient restriction endonuclease sites, and one or more selectable markers. The term “vector” should also be construed to include non-plasmid and non-viral compounds which facilitate transfer of nucleic acid into cells, such as, for example, polylysine compounds, liposomes, and the like.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the modified immune cell in vitro or ex vivo. A number of retroviral systems are known in the art. In some embodiments, adenovirus vectors are used. In some embodiments, lentivirus vectors are used. In some embodiments, self-inactivating lentiviral vectors are used. For example, self-inactivating lentiviral vectors can be packaged with protocols known in the art. The resulting lentiviral vectors can be used to transduce a mammalian cell (such as human T cells) using methods known in the art.

In some embodiments, the vector is a non-viral vector, such as a plasmid, or an episomal expression vector.

In some embodiments, the vector is an expression vector. “Expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells they comprise normally promoters ensuring initiation of transcription and optionally poly-A signals ensuring termination of transcription and stabilization of the transcript. Examples of regulatory elements permitting expression in eukaryotic host cells are AOX1 or GAL1 promoter in yeast or the CMV-, SV40-, RSV-promoter (Rous sarcoma virus), CMV-enhancer, SV40-enhancer or a globin intron in mammalian and other animal cells. Furthermore, depending on the expression system used signal peptides (e.g., leader sequences) capable of directing the polypeptide to a cellular compartment or secreting it into the medium may be added to the coding sequence of the recited nucleic acid sequence and are well known in the art. The signal peptides(s) is (are) assembled in appropriate phase with translation, initiation and termination sequences, and preferably, a signal peptide capable of directing secretion of translated protein, or a portion thereof, into the periplasmic space or extracellular medium. Optionally, the nucleic acid sequence can encode a fusion protein including an N-terminal identification peptide imparting desired characteristics, e.g., stabilization or simplified purification of expressed recombinant product. Suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al., Cancer Immunol Immunother (2001) 50(3), 141-150) or pSPORT1 (GIBCO BRL).

### Methods of Preparation

The present application also provides methods of preparing any one of the modified immune cells described herein.

In some embodiments, there is provided a method of producing a modified immune cell, comprising: introducing into a precursor immune cell a first nucleic acid encoding the first polypeptide and optionally a second nucleic acid encoding the second polypeptide. In some embodiments, the precursor immune cell is selected from the group consisting of a cytotoxic T cell, a helper T cell, a natural killer (NK) cell, an NK-T cell, an iNK-T cell, an NK-T like cell, an αβT cell and a γδT cell. In some embodiments, the precursor immune cell is a cytotoxic T cell. In some embodiments, the precursor immune cell is a γδT cell. In some embodiments, the precursor immune cell is a tumor-infiltrating T cell or DC-activated T cell. In some embodiments, the precursor immune cell comprises any one of the engineered receptors described herein. In some embodiments, the method further comprises introducing into the precursor immune cell a third nucleic acid encoding any one of the engineered receptors described herein.

In some embodiments, the engineered receptor is a chimeric antigen receptor (CAR). In some embodiments, the engineered receptor is a modified T-cell receptor (TCR). In some embodiments, the engineered receptor is a T-cell antigen coupler (TAC) receptor. In some embodiments, the first nucleic acid sequence, the optional second nucleic acid sequence, and the third nucleic acid sequence are operably linked to the same promoter. In some embodiments, the first nucleic acid sequence, the optional second nucleic acid sequence, and the third nucleic acid sequence are operably linked to separate promoters. In some embodiments, the first nucleic acid and the optional second nucleic acid, and/or the third nucleic acid sequence are on the same vector. In some embodiments, the first nucleic acid and the optional second nucleic acid, and/or the third nucleic acid sequence are on separate vectors. In some embodiments, the vector is a viral vector. In some embodiments, the viral vector is selected from the group consisting of an adenoviral vector, an adeno-associated virus vector, a retroviral vector, a lentiviral vector, a herpes simplex viral vector, and derivatives thereof. In some embodiments, the vector is a non-viral vector. In some embodiments, the vector is an episomal expression vector. In some embodiments, the method further comprises isolating or enriching immune cells comprising the first nucleic acid sequence and/or the second nucleic acid sequence. In some embodiments, the method further comprises formulating the modified immune cells with at least one pharmaceutically acceptable carrier.

In some embodiments, there is provided an isolated host cell comprising any one of the nucleic acids or vectors described herein. The host cells may be useful in expression or cloning of the first polypeptides, the second polypeptides, and/or the engineered receptors, nucleic acids or vectors encoding the first polypeptides, the second polypeptides, and/or the engineered receptors. Suitable host cells can include, without limitation, prokaryotic cells, fungal cells, yeast cells, or higher eukaryotic cells such as mammalian cells. In some embodiments, the host cells comprise a first vector encoding a first polypeptide, a second vector encoding a second polypeptide, and a third vector encoding a third polypeptide (e.g., an engineered receptor). In some embodiments, the host cells comprise a single vector comprising isolated nucleic acids encoding a first polypeptide, a second polypeptide, and a third polypeptide.

The precursor immune cells can be prepared using a variety of methods known in the art. For example, primary immune cells, such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, immune cells (such as T cells) can be obtained from a unit of blood collected from an individual using any number of techniques known in the art, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS), or a wash solution lacking divalent cations, such as calcium and magnesium. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, primary T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA, and CD45RO cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells.

In some embodiments, a T cell population may further be enriched by negative selection using a combination of antibodies directed to surface markers unique to the negatively selected cells. For example, one method involves cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD1lb, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar methods of selection.

Methods of introducing vectors or nucleic acids into a host cell (such as a precursor immune cell) are known in the art. The vectors or nucleic acids can be transferred into a host cell by physical, chemical, or biological methods.

Physical methods for introducing the vector(s) or nucleic acid(s) into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the vector(s) or nucleic acid(s) into a host cell include the use of DNA and RNA vectors. Viral vectors have become the most widely used method for inserting genes into mammalian, e.g., human cells.

Chemical means for introducing the vector(s) or nucleic acid(s) into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the transduced or transfected precursor immune cell is propagated ex vivo after introduction of the heterologous nucleic acid(s). In some embodiments, the transduced or transfected precursor immune cell is cultured to propagate for at least about any of 1 day, 2 days, 3 days, 4 days, 5 days, 6 days, 7 days, 10 days, 12 days, or 14 days. In some embodiments, the transduced or transfected precursor immune cell is cultured for no more than about any of 1 day, 2 days, 3 days, 4 days, 5 days, 6 days, 7 days, 10 days, 12 days, or 14 days. In some embodiments, the transduced or transfected precursor immune cell is further evaluated or screened to select the modified immune cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)).

Other methods to confirm the presence of the heterologous nucleic acid(s) in the precursor immune cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

### III. Methods of Treatment

One aspect of the present application relates to methods of treating a disease or condition (e.g., cancer) in an individual, comprising administering to the individual an effective amount of any one of the modified immune cells described herein. The present application contemplates modified immune cells that can be administered either alone or in any combination with another therapy, and in at least some aspects, together with a pharmaceutically acceptable carrier or excipient. In some embodiments, prior to administration, the modified immune cells may be combined with suitable pharmaceutical carriers and excipients that are well known in the art.

In some embodiments, there is provided a method of treating cancer (e.g., solid cancer) in an individual (e.g., human), comprising administering to the individual an effective amount of a pharmaceutical composition comprising a modified immune cell (e.g., a CAR-T cell) and a pharmaceutically acceptable carrier, wherein the modified immune cell comprises: a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein upon binding of the first target binding domain and second target binding domain to their corresponding target, the first TLR signaling domain and the second TLR signaling domain associate with each other to form a TLR signaling moiety capable of inducing TLR signaling, and wherein the first target binding domain and the second target binding domain each binds to a subunit of a multimeric target molecule. In some embodiments, the first target binding domain and the second binding domain bind to the same target molecule. In some embodiments, the first target binding domain and the second binding domain each binds to the same target site on the target molecule. In some embodiments, the modified immune cell further comprises an engineered receptor, such as a chimeric antigen receptor (CAR), an engineered TCR, or a T-cell antigen coupler (TAC) receptor. In some embodiments, the modified immune cell comprises a CAR system (e.g., a CAR fusion construct), wherein the CAR fusion construct comprises a CAR and TLR polypeptides (e.g., two or more TLR polypeptides fused to the CAR). In some embodiments, the modified immune cell comprises a CAR system (e.g., a CAR fusion construct), wherein the CAR fusion construct comprises an anti-CD19 CAR and anti-CD20 TLR polypeptides. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD20 scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1. In some embodiments the CAR fusion construct comprises SEQ ID NO: 1. In some embodiments, the modified immune cell comprises a CAR system (e.g., a CAR fusion construct), wherein the CAR fusion construct comprises an anti-CD19 CAR and anti-NKG2D TLR polypeptides. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 59. In some embodiments the CAR fusion construct comprises SEQ ID NO: 59. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 60. In some embodiments the CAR fusion construct comprises SEQ ID NO: 60. In some embodiments, the modified immune cell comprises a CAR system (e.g., a CAR fusion construct), wherein the CAR fusion construct comprises an anti-GPC3 CAR and anti-NKG2D TLR polypeptides. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 57. In some embodiments the CAR fusion construct comprises SEQ ID NO: 57. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 58. In some embodiments the CAR fusion construct comprises SEQ ID NO: 58. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-BCMA TLR polypeptides. In some embodiments, the CAR fusion construct comprises a single anti-BCMA CAR and anti-BCMA TLR polypeptides. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 62. In some embodiments the CAR fusion construct comprises SEQ ID NO: 62. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 63. In some embodiments the CAR fusion construct comprises SEQ ID NO: 63. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 64. In some embodiments the CAR fusion construct comprises SEQ ID NO: 64. In some embodiments, the CAR fusion construct comprises a tandem anti-BCMA CAR and anti-BCMA TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a tandem anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 61. In some embodiments the CAR fusion construct comprises SEQ ID NO: 61. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-GPRC5D TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 65. In some embodiments the CAR fusion construct comprises SEQ ID NO: 65. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 72. In some embodiments the CAR fusion construct comprises SEQ ID NO: 72. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 73. In some embodiments the CAR fusion construct comprises SEQ ID NO: 73. In some embodiments, the modified immune cell comprises a CAR system (e.g., a CAR fusion construct), wherein the CAR fusion construct comprises an anti-CLL1 CAR and anti-CD33 (e.g., anti-CD33 V domain and/or anti-CD33 C2 domain) TLR polypeptides. In some embodiments, the CAR fusion construct comprises, from the N-terminus to the C-terminus: a signal peptide, an anti-CLL1 sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD33 sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 71. In some embodiments the CAR fusion construct comprises SEQ ID NO: 71. In some embodiments, the CAR fusion construct comprises, from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain, a P2A cleavage site, a signal peptide, an anti-CD33 V domain sdAb, a TLR2 transmembrane domain, a TLR2 signaling domain (e.g., the cytoplasmic portion of TLR2), a P2A cleavage site, a signal peptide, an anti-CD33 C2 domain sdAb, a TLR1 transmembrane domain, and a TLR1 signaling domain (e.g., the cytoplasmic portion of TLR1). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 3. In some embodiments the CAR fusion construct comprises SEQ ID NO: 3. In some embodiments, the first polypeptide further comprises an intracellular domain of a first cytokine receptor, and/or the second polypeptide further comprises an intracellular domain of a second cytokine receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a method of treating cancer (e.g., solid cancer) in an individual (e.g., human), comprising administering to the individual an effective amount of a pharmaceutical composition comprising a modified immune cell (e.g., a CAR-T cell) and a pharmaceutically acceptable carrier, wherein the modified immune cell comprises: a) a first polypeptide comprising: i) a first target binding domain, ii) a first TLR transmembrane domain, and iii) a first TLR signaling domain; and b) a second polypeptide comprising: i) a second target binding domain, ii) a second TLR transmembrane domain, and iii) a second TLR signaling domain, wherein the first target binding domain and the second binding domain bind to the same target molecule, and wherein the first target binding domain and the second target binding domain each binds to a different non-overlapping target sites on a single target molecule, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the modified immune cell further comprises an engineered receptor, such as a chimeric antigen receptor (CAR), an engineered TCR, or a T-cell antigen coupler (TAC) receptor. In some embodiments, the modified immune cell comprises a CAR system (e.g., a CAR fusion construct), wherein the CAR fusion construct comprises a CAR and TLR polypeptides (e.g., two or more TLR polypeptides fused to the CAR). In some embodiments, the modified immune cell comprises a CAR system (e.g., a CAR fusion construct), wherein the CAR fusion construct comprises an anti-CD19 CAR and anti-CD20 TLR polypeptides. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD20 scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1. In some embodiments the CAR fusion construct comprises SEQ ID NO: 1. In some embodiments, the modified immune cell comprises a CAR system (e.g., a CAR fusion construct), wherein the CAR fusion construct comprises an anti-CD19 CAR and anti-NKG2D TLR polypeptides. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 59. In some embodiments the CAR fusion construct comprises SEQ ID NO: 59. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 60. In some embodiments the CAR fusion construct comprises SEQ ID NO: 60. In some embodiments, the modified immune cell comprises a CAR system (e.g., a CAR fusion construct), wherein the CAR fusion construct comprises an anti-GPC3 CAR and anti-NKG2D TLR polypeptides. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 57. In some embodiments the CAR fusion construct comprises SEQ ID NO: 57. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 58. In some embodiments the CAR fusion construct comprises SEQ ID NO: 58. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-BCMA TLR polypeptides. In some embodiments, the CAR fusion construct comprises a single anti-BCMA CAR and anti-BCMA TLR polypeptides. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 62. In some embodiments the CAR fusion construct comprises SEQ ID NO: 62. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 63. In some embodiments the CAR fusion construct comprises SEQ ID NO: 63. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 64. In some embodiments the CAR fusion construct comprises SEQ ID NO: 64. In some embodiments, the CAR fusion construct comprises a tandem anti-BCMA CAR and anti-BCMA TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a tandem anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 61. In some embodiments the CAR fusion construct comprises SEQ ID NO: 61. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-GPRC5D TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a anti-GPRC5D scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 65. In some embodiments the CAR fusion construct comprises SEQ ID NO: 65. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 72. In some embodiments the CAR fusion construct comprises SEQ ID NO: 72. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 73. In some embodiments the CAR fusion construct comprises SEQ ID NO: 73. In some embodiments, the modified immune cell comprises a CAR system (e.g., a CAR fusion construct), wherein the CAR fusion construct comprises an anti-CLL1 CAR and anti-CD33 (e.g., anti-CD33 V domain and/or anti-CD33 C2 domain) TLR polypeptides. In some embodiments, the CAR fusion construct comprises, from the N-terminus to the C-terminus: a signal peptide, an anti-CLL1 sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD33 sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 71. In some embodiments the CAR fusion construct comprises SEQ ID NO: 71. In some embodiments, the CAR fusion construct comprises, from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain, a P2A cleavage site, a signal peptide, an anti-CD33 V domain sdAb, a TLR2 transmembrane domain, a TLR2 signaling domain (e.g., the cytoplasmic portion of TLR2), a P2A cleavage site, a signal peptide, an anti-CD33 C2 domain sdAb, a TLR1 transmembrane domain, and a TLR1 signaling domain (e.g., the cytoplasmic portion of TLR1). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 3. In some embodiments, the first polypeptide further comprises an intracellular domain of a first cytokine receptor, and/or the second polypeptide further comprises an intracellular domain of a second cytokine receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, there is provided a method of treating cancer (e.g., solid cancer) in an individual (e.g., human), comprising administering to the individual an effective amount of a pharmaceutical composition comprising a modified immune cell (e.g., a CAR-T cell) and a pharmaceutically acceptable carrier, wherein the modified immune cell comprises: a) a first polypeptide comprising: i) a first target binding domain, ii) a first TLR transmembrane domain, and iii) a first TLR signaling domain; b) a second polypeptide comprising: i) a second target binding domain, ii) a second TLR transmembrane domain, and iii) a second TLR signaling domain; and c) an engineered receptor, wherein the engineered receptor comprises an extracellular domain specifically recognizing the same target molecule as the first polypeptide and/or the second polypeptide, wherein upon binding of the first target binding domain and second target binding domain to their corresponding target, the first TLR signaling domain and the second TLR signaling domain associate with each other to form a TLR signaling moiety capable of inducing TLR signaling. In some embodiments, the engineered receptor comprises an extracellular domain specifically recognizing a non-overlapping target site on the same target molecule as the first polypeptide and/or the second polypeptide. In some embodiments, the engineered receptor is a chimeric antigen receptor (CAR), an engineered TCR, or a T-cell antigen coupler (TAC) receptor. In some embodiments, the modified immune cell comprises a CAR system (e.g., a CAR fusion construct), wherein the CAR fusion construct comprises a CAR and TLR polypeptides (e.g., two or more TLR polypeptides fused to the CAR). In some embodiments, the modified immune cell comprises a CAR system (e.g., a CAR fusion construct), wherein the CAR fusion construct comprises an anti-CD19 CAR and anti-CD20 TLR polypeptides. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD20 scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1. In some embodiments the CAR fusion construct comprises SEQ ID NO: 1. In some embodiments, the modified immune cell comprises a CAR system (e.g., a CAR fusion construct), wherein the CAR fusion construct comprises an anti-CD19 CAR and anti-NKG2D TLR polypeptides. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 59. In some embodiments the CAR fusion construct comprises SEQ ID NO: 59. In some embodiments, the CAR fusion construct comprises, from the N-terminus to the C-terminus: a signal peptide, an anti-CD19 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 60. In some embodiments the CAR fusion construct comprises SEQ ID NO: 60. In some embodiments, the modified immune cell comprises a CAR system (e.g., a CAR fusion construct), wherein the CAR fusion construct comprises an anti-GPC3 CAR and anti-NKG2D TLR polypeptides. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 57. In some embodiments the CAR fusion construct comprises SEQ ID NO: 57. In some embodiments, the CAR fusion construct comprises, from the N-terminus to the C-terminus: a signal peptide, an anti-GPC3 scFv, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-NKG2D ECD, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 58. In some embodiments the CAR fusion construct comprises SEQ ID NO: 58. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-BCMA TLR polypeptides. In some embodiments, the CAR fusion construct comprises a single anti-BCMA CAR and anti-BCMA TLR polypeptides. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 62. In some embodiments the CAR fusion construct comprises SEQ ID NO: 62. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 63. In some embodiments the CAR fusion construct comprises SEQ ID NO: 63. In some embodiments, the CAR fusion construct comprises, from the N-terminus to the C-terminus: a signal peptide, a single anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a single anti-BCMA sdAb, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 64. In some embodiments the CAR fusion construct comprises SEQ ID NO: 64. In some embodiments, the CAR fusion construct comprises a tandem anti-BCMA CAR and anti-BCMA TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, a tandem anti-BCMA sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 61. In some embodiments the CAR fusion construct comprises SEQ ID NO: 61. In some embodiments, the modified immune cell comprises a CAR system (e.g., a CAR fusion construct), wherein the CAR fusion construct comprises an anti-BCMA CAR (e.g., a single anti-BCMA CAR or a tandem anti-BCMA CAR) and anti-GPRC5D TLR polypeptides. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 65. In some embodiments the CAR fusion construct comprises SEQ ID NO: 65. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD8α hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 72. In some embodiments the CAR fusion construct comprises SEQ ID NO: 72. In some embodiments, the CAR fusion construct comprises, from the N-terminus to the C-terminus: a signal peptide, a tandem anti-BCMA sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-GPRC5D scFv, a CD28a hinge domain, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 73. In some embodiments the CAR fusion construct comprises SEQ ID NO: 73. In some embodiments, the modified immune cell comprises a CAR system (e.g., a CAR fusion construct), wherein the CAR fusion construct comprises an anti-CLL1 CAR and anti-CD33 (e.g., anti-CD33 V domain and/or anti-CD33 C2 domain) TLR polypeptides. In some embodiments, the CAR fusion construct comprises, from the N-terminus to the C-terminus: a signal peptide, an anti-CLL1 sdAb, a CD8α hinge domain, a CD8α transmembrane (TM) domain, the cytoplasmic portion of the 4-1BB (CD137) co-stimulatory signaling domain, and a CD3ζ primary intracellular signaling domain, a P2A cleavage site, a signal peptide, an anti-CD33 sdAb, a TLR4 transmembrane (TM) region, and the cytoplasmic portion of TLR4 (e.g., TLR4 primary intracellular signaling domain). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 71. In some embodiments the CAR fusion construct comprises SEQ ID NO: 71. In some embodiments, the CAR fusion construct comprises, from the N-terminus to the C-terminus: a signal peptide, an anti-CLL1 sdAb, a CD28a hinge domain, a CD28a transmembrane (TM) domain, the cytoplasmic portion of the CD28 co-stimulatory signaling domain, and a CD3ζ primary intracellular signaling domain, a P2A cleavage site, a signal peptide, an anti-CD33 V domain sdAb, a TLR2 transmembrane domain, a TLR2 signaling domain (e.g., the cytoplasmic portion of TLR2), a P2A cleavage site, a signal peptide, an anti-CD33 C2 domain sdAb, a TLR1 transmembrane domain, and a TLR1 signaling domain (e.g., the cytoplasmic portion of TLR1). In some embodiments, the CAR fusion construct comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 3. In some embodiments, the CAR fusion construct comprises SEQ ID NO: 3. In some embodiments, the first polypeptide further comprises an intracellular domain of a first cytokine receptor, and/or the second polypeptide further comprises an intracellular domain of a second cytokine receptor. In some embodiments, the modified immune cell is selected from the group consisting of a cytotoxic T cell, a helper T cell, a natural killer (NK) cell, an NK-cell, an iNK-T cell, an NK-T like cell, an αβT cell and a γδT cell.

In some embodiments, the method of treating cancer has one or more of the following biological activities: (1) killing cancer cells; (2) inhibiting proliferation of cancer cells; (3) inducing redistribution of peripheral T cells; (4) inducing immune response in a tumor; (5) reducing tumor size; (6) alleviating one or more symptoms in an individual having cancer; (7) inhibiting tumor metastasis; (8) prolonging survival; (9) prolonging time to cancer progression; (10) preventing, inhibiting, or reducing the likelihood of the recurrence of a cancer; (11) improving quality of life of the individual; (12) facilitating T cell infiltration in tumors, and (13) reducing incidence or burden of preexisting tumor metastasis (such as metastasis to the lymph node). In some embodiments, the method achieves a tumor cell death rate of at least about any of 40%, 50%, 60%, 70%, 80%, 90%, 95%, or more. In some embodiments, the method reduces at least about 10% (including for example at least about any of 20%, 30%, 40%, 60%, 70%, 80%, 90%, or 100%) of the tumor size. In some embodiments, the method inhibits at least about 10% (including for example at least about any of 20%, 30%, 40%, 60%, 70%, 80%, 90%, or 100%) of the metastasis. In some embodiments, the method prolongs the survival of the individual by at least any of 1, 2, 3, 4, 5, 6, 7, 8, 9, 10, 11, 12, 18, 24, or more months. In some embodiments, the method prolongs the time to cancer progression by at least any of 1, 2, 3, 4, 5, 6, 7, 8, 9, 10, 11, 12, 18, 24, or more months.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, hormone therapy, radiation, gene therapy, immunotherapy (such as T cell therapy), bone marrow transplantation, stem cell transplantation, targeted therapy, cryotherapy, ultrasound therapy, photodynamic therapy, radio-frequency ablation or the like, in an adjuvant setting or a neoadjuvant setting (i.e., the method may be carried out before the primary/definitive therapy). In some embodiments, the method is used to treat an individual who has previously been treated. In some embodiments, the cancer has been refractory to prior therapy. In some embodiments, the method is used to treat an individual who has not previously been treated.

In some embodiments, the individual has a low tumor burden. Tumor burden for solid tumor can be measured according to the Response Evaluation Criteria in Solid Tumors (RECIST) 1.1 guideline. See, Eisenhauer E A et al., European Journal of Cancer 45 (2009) 228-247. For example, tumor burden can be assessed for measurable tumors at baseline of treatment based on: (1) tumor lesions (e.g., by CT scan, caliper measurement by clinical exam, and/or chest X-ray) and (2) malignant lymph nodes. For example, tumor burden for solid cancer can be quantified as the sum of the diameters of 5 target lesions, with a maximum of 2 per organ. Tumor burden for liquid cancer can be measured as the sum of product diameters of up to 6 index lesions according to Cheson 2007 criteria assessed by a radiologist. See, Cheson B D et al., J. Clin. Oncol., 2007; 25(5): 579-586. In some embodiments, an individual with a low tumor burden has a tumor burden of no more than about any one of 4×10³, 3×10³, 2×10³, 1×10³, 5×10², 2×10², 1×10² or less mm².

In some embodiments, the individual does not experience Grade 3 or Grade 4 adverse side effects after receiving the treatment. Grading of adverse events are according to Common Terminology Criteria for Adverse Events v3.0 (CTCAE). In some embodiments, the individual does not experience cytokine storm after receiving the treatment.

The effective amount of the modified immune cells administered in the methods described herein will depend upon a number of factors, such as the particular type and stage of cancer being treated, the route of administrations, the activity of the first polypeptide, the second polypeptide, and/or the engineered receptors, and the like. Appropriate dosage regimen can be determined by a physician based on clinical factors, including the patient's size, body surface area, age, the particular compound to be administered, sex, time and route of administration, general health, and other drugs being administered concurrently. In some embodiments, that effective amount of the pharmaceutical composition is below the level that induces a toxicological effect (i.e., an effect above a clinically acceptable level of toxicity) or is at a level where a potential side effect can be controlled or tolerated when the pharmaceutical composition is administered to the individual. In some embodiments, the effective amount of the pharmaceutical composition comprises about 10⁵ to about 10¹⁰ modified immune cells.

In some embodiments, the pharmaceutical composition is administered for a single time (e.g. bolus injection). In some embodiments, the pharmaceutical composition is administered for multiple times (such as any of 2, 3, 4, 5, 6, or more times). If multiple administrations, they may be performed by the same or different routes and may take place at the same site or at alternative sites. The pharmaceutical composition may be administered at a suitable frequency, such as from daily to once per year. The optimal dosage and treatment regime for a particular patient can readily be determined by one skilled in the art of medicine by monitoring the patient for signs of disease and adjusting the treatment accordingly.

In some embodiments, the individual to be treated is a mammal. Examples of mammals include, but are not limited to, humans, monkeys, rats, mice, hamsters, guinea pigs, dogs, cats, rabbits, pigs, sheep, goats, horses, cattle and the like. In some embodiments, the individual is a human.

### Pharmaceutical Compositions

Further provided by the present application are pharmaceutical compositions comprising any one of the modified immune cells described herein, and optionally a pharmaceutically acceptable carrier.

The pharmaceutical composition of the present applicant may comprise any number of the modified immune cells. In some embodiments, the pharmaceutical composition comprises a single copy of the modified immune cell. In some embodiments, the pharmaceutical composition comprises at least about any of 1, 10, 100, 1000, 10⁴, 10⁵, 10⁶, 10⁷, 10⁸ or more copies of the modified immune cells. In some embodiments, the pharmaceutical composition comprises a single type of modified immune cell. In some embodiments, the pharmaceutical composition comprises at least two types of modified immune cells, wherein the different types of modified immune cells differ by their cell sources, cell types, expressed chimeric receptors, and/or promoters, etc.

“Carriers” as used herein include pharmaceutically acceptable carriers, excipients, or stabilizers which are nontoxic to the cells or individual being exposed thereto at the dosages and concentrations employed. Often the physiologically acceptable carrier is an aqueous pH buffered solution. Examples of suitable pharmaceutical carriers are well known in the art and include phosphate buffered saline solutions, water, emulsions, such as oil/water emulsions, various types of wetting agents, sterile solutions, etc. Acceptable carriers, excipients, or stabilizers are nontoxic to recipients at the dosages and concentrations employed.

Pharmaceutical compositions comprising such carriers can be formulated by well-known conventional methods. The solvent or diluent is preferably isotonic, hypotonic or weakly hypertonic and has a relatively low ionic strength. Representative examples include sterile water, physiological saline (e.g. sodium chloride), Ringer's solution, glucose, trehalose or saccharose solutions, Hank's solution, and other aqueous physiologically balanced salt solutions (see, for example, the most current edition of Remington: The Science and Practice of Pharmacy, A. Gennaro, Lippincott, Williams&Wilkins).

The pharmaceutical compositions described herein may be administered via any suitable routes. In some embodiments, the pharmaceutical composition is administered parenterally, transdermally (into the dermis), intraluminally, intra-arterially (into an artery), intramuscularly (into muscle), intrathecally or intravenously. In some embodiments, the pharmaceutical composition is administered subcutaneously (under the skin). In some embodiments, the pharmaceutical composition is administered intravenously. In some embodiments, the pharmaceutical composition is administered to the individual via infusion or injection. In some embodiments, the pharmaceutical composition is administered directly to the target site, e.g., by biolistic delivery to an internal or external target site or by catheter to a site in an artery. In some embodiments, the pharmaceutical composition is administered locally, e.g., intratumorally. Administrations may use conventional syringes and needles or any compound or device available in the art capable of facilitating or improving delivery of the active agent(s) in the subject.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. Various virus formulation are available in the art either in frozen, liquid form or lyophilized form (e.g. WO98/02522, WO01/66137, WO03/053463, WO2007/056847 and WO2008/114021, etc.). Solid (e.g. dry powdered or lyophilized) compositions can be obtained by a process involving vacuum drying and freeze-drying (see e.g. WO2014/053571). It is envisaged that the pharmaceutical composition of the disclosure might comprise, in addition to the modified immune cells described herein, further biologically active agents, depending on the intended use of the pharmaceutical composition.

In some embodiments, the pharmaceutical composition is suitably buffered for human use. Suitable buffers include without limitation phosphate buffer (e.g. PBS), bicarbonate buffer and/or Tris buffer capable of maintaining a physiological or slightly basic pH (e.g. from approximately pH 7 to approximately pH 9). In some embodiments, the pharmaceutical composition can also be made to be isotonic with blood by the addition of a suitable tonicity modifier, such as glycerol.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the modified immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the modified immune cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the modified immune cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### IV. Kits and Articles of Manufacture

Also provided are kits, unit dosages, and articles of manufacture comprising any one of the modified immune cells, or the compositions (e.g. pharmaceutical composition) described herein. In some embodiments, a kit is provided which contains any one of the pharmaceutical compositions described herein and preferably provides instructions for its use. In some embodiments, the kit, in addition to the modified immune cell, further comprises a second cancer therapy, such as chemotherapy, hormone therapy, and/or immunotherapy. The kit(s) may be tailored to a particular cancer for an individual and comprise respective second cancer therapies for the individual.

The kits may contain one or more additional components, such as containers, reagents, culturing media, inducers, cytokines, buffers, antibodies, and the like to allow propagation or induction of the modified immune cell. The kits may also contain a device for local administration (such as intratumoral injection) of the pharmaceutical composition to a tumor site.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like. Some components of the kits may be packaged either in aqueous media or in lyophilized form.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the disclosure and should therefore not be considered to limit the disclosure in any way. The following examples and detailed description are offered by way of illustration and not by way of limitation.

### Example 1: Preparation of CAR-T Cells Expressing Exogenously Introduced CD19 and CD20 Toll-Like Receptor (TLR) Domains

This example shows the construction of exemplary armored CAR-T cells expressing exogenously introduced toll-like receptor (TLR) domains. In particular, this example shows the construction of CD19 and/or CD20 expressing CAR-T cells.

### 1.1. Construction of Chimeric Antigen Receptors (CARs)

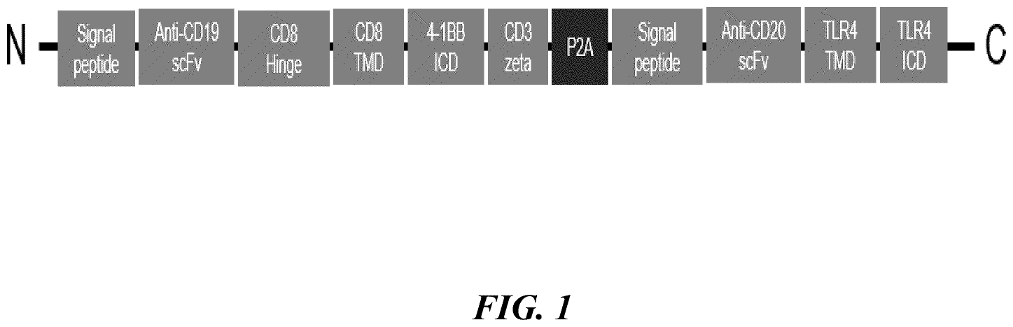

To construct a CD19-co-CD20 CAR (SEQ ID NO: 1), a CAR backbone sequence encoding a CAR backbone polypeptide comprising from the N-terminus to the C-terminus: a signal peptide (SEQ ID NO: 5), anti-CD19 scFv (SEQ ID NO: 6), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), was codon optimized and chemically synthesized. The sequence of an inducible costimulatory molecule comprising from the N-terminus to the C-terminus: a signal peptide (SEQ ID NO: 5), an anti-CD20 scFv (SEQ ID NO: 11), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24). This construct is shown in FIG. 1. The CAR backbone and costimulatory molecule were cloned into a lentiviral vector (e.g., a second generation lentiviral vector), downstream and operably linked to a constitutive hEF1α promoter for in vitro transcription. Transient retroviral supernatants were produced refer as exemplified in Blood (2006) 108 (12): 3890-3897.

A second CD19 CAR backbone comprising 4-1BB as costimulatory domain (“CD19 BM CAR”), comprising from the N-terminus to the C-terminus: a signal peptide (SEQ ID NO: 5), anti-CD19 scFv (SEQ ID NO: 6), a CD8α hinge domain (SEQ ID NO: 7), a CD8α transmembrane (TM) domain (SEQ ID NO: 8), a 4-1BB co-stimulatory signaling domain (SEQ ID NO: 9), and a CD3ζ primary intracellular signaling domain (SEQ ID NO: 10), was prepared as described above and used for comparative analysis.

### 1.2. Construction of CAR-T Cells

### Viral Preparation

Lentivirus packing plasmid mixture including pCMV-ΔR-8.47 and pMD2.G was purchased from Addgene, and admixed with the appropriate CAR-encoding plasmid at a pre-optimized ratio with polyethylenimine. HEK293 cells were transfected with the mixture of lentivirus and CAR-constructs, and were cultured overnight. Following overnight culture the supernatant was collected. The supernatant was centrifuged to further remove cellular debris, and filtered through a 0.45 μm PES filter. The virus particles were pelleted, and rinsed with pre-chilled DPBS. The virus was aliquoted and stored at −80° C. immediately, and the virus titer was determined by measuring supT1 cell line transduction efficiency by flow cytometric assay.

### Virus Transduction of T Cells

Leukocytes were collected from healthy donors by apheresis. Peripheral blood mononuclear cells (PBMCs) were isolated using FICOLL-PAQUE™ PLUS Media, and human T cells were purified from PMBCs using a Pan T cell isolation kit (Miltenyi). The purified T cells were subsequently pre-activated for 48 hours with a human T cell activation/expansion kit (Miltenyi). Following pre-activation, anti-CD3/CD28 MACSiBead™ particles were added at a bead-to-cell ratio of 1:2 to expand the T cells.

The pre-activated T cells were transduced with lentivirus in the presence of 8 μg/mL polybrene. The cells were cultured in 6-well tissue culture plates with 4×10⁶ T cells/well, for approximately 48 hours at 37° C. The transduced cells were centrifuged, the supernatant was removed, and the pelleted cells were resuspended at 0.5×10⁶ cells/mL in fresh media supplemented with 300 IU/ml IL-2 for culture. The cell concentration was adjusted to 0.5×10⁶ cells/mL every 2 to 3 days.

CAR expression was detected on T cells by admixing protein L and rabbit-anti-sdAb with the CAR cultured cells, to detect the cell surface scFvs and sdAbs, respectively.

### Example 2: In Vitro Cytotoxicity Evaluations of CD19-Co-CD20 CAR-T Cells

This example shows the in vitro anti-tumor activity of CD19-co-CD20 CAR-T cells.

### 2.1 Lactate Dehydrogenase (LDH) Cytoxicity

For quick evaluation of anti-tumor activities of engineered T cells (e.g., CD19 BM CAR-T and CD19-co-CD20 CAR-T cells) in vitro, a lactate dehydrogenase (LDH) assay for cytotoxicity was performed. On day 5 or day 9 post-transduction, CD19-co-CD20 CAR transduced T cells (SEQ ID NO: 1) were harvested and co-incubated with target cells (CD19 and CD20-expressing BLL tumor Raji cells) at E/T ratio (Effector: CAR-T/Target) ratio of 1:1, 1:0.3, for 20 hours, respectively. CD19 BM CAR-T cells (SEQ ID NO: 2) were used as a positive control, untransduced T cells (“UnT”) from the same batch were used as a negative control. The assay was performed following the manufacturer's manual (Roche).

The in vitro cytotoxicity was calculated by the equation below ([LDH]_{E+T}: the LDH released from E/T co-incubation, [LDH]_{E}: the LDH released from Effector only, [LDH]ₘₐₓ: the LDH released from target cells treated with Triton X-100, [LDH]ₘᵢₙ: the LDH released from UnTreated target cells):

$$Cytotoxicity \% = \frac{{[ LDH ]}_{E + T} - {[ LDH ]}_{E} - {[ LDH ]}_{min}}{{[ LDH ]}_{max} - {[ LDH ]}_{min}} \times 100 \%$$

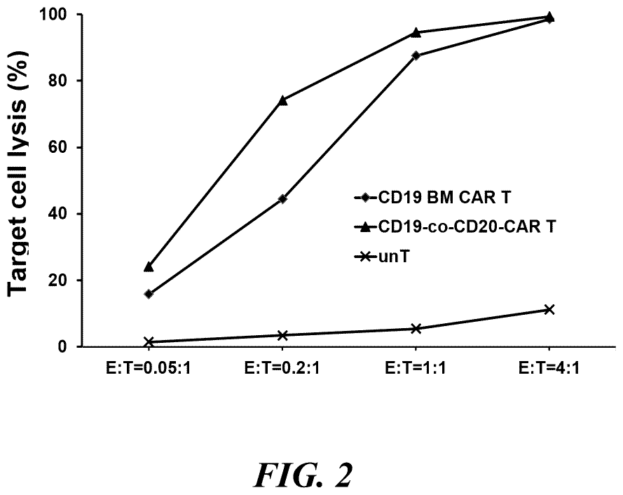

All tested CAR constructs (e.g., CD19-co-CD20 CAR-T, CD19 BM CAR-T, and UnT cells) effectively transduced human T cells with CAR expression rates between 25% and 60%. Cell growth and viability of transduced cells were not affected relative to untransduced T cells in the same batch of experiments. As shown in FIG. 2, CD19 BM CAR-T and CD19-co-CD20 CAR-T cells induced target cell lysis in a dose dependent manner in vitro. Moreover, CD19-co-CD20 CAR-T cells exhibited stronger cytotoxicity against target cells than CD19 BM CAR-T at low E/T ratios.

### 2.2 Cytokine Secretion by Homogeneous Time Resolved Fluorescence (HTRF)

Another measure of effector T-cell activation and proliferation is the production of effector cytokines such as IFNγ and TNFα. Supernatants from the in vitro cytotoxicity assay were collected to assess CAR-induced cytokine release, and Homogeneous Time Resolved Fluorescence (HTRF) assays for IFN-γ (Cisbio) were performed according to the manufacturer's manual.

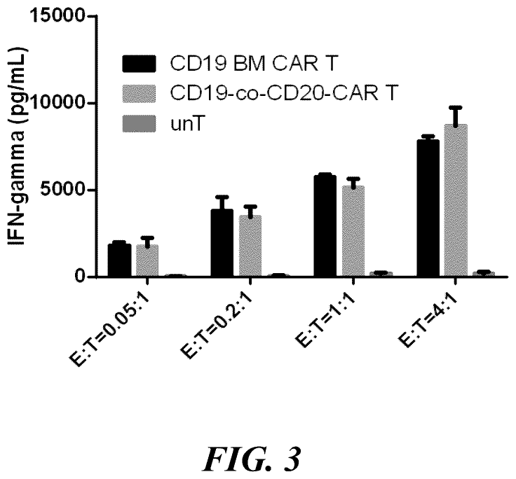

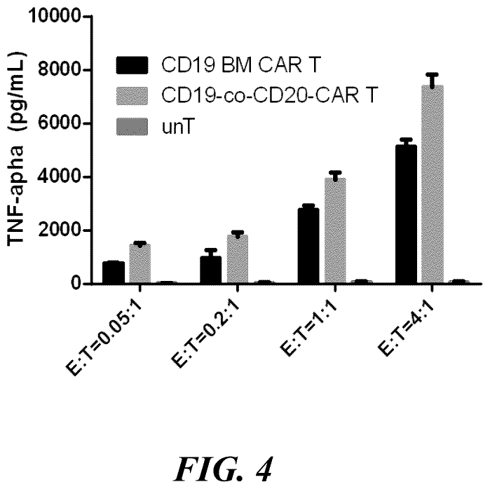

CAR-T cells (e.g., CD19 BM CAR-T, CD19-co-CD20 CAR-T, and untransduced “unT” CAR-T cells) were co-cultured with Raji target cells. The culture supernatants were collected after 24 h to assess IFNγ and TNFα release as a measure of T cell activation. As shown in FIGS. 3 and 4, CD19-co-CD20 CAR-T cells co-cultured with target cells secreted significant amounts of TNFα and IFNγ. Moreover, the level of TNFα secretion was higher than that of positive control CD19 BM CAR-T cells.

### 2.3 Long-Term Co-Culture Assay

To evaluate the long-term killing efficacy of CD19-co-CD20 CAR-T cells, long-term co-culture assays were performed to mimic the dynamic killing process in vivo. Transduced (e.g., CD19 BM CAR-T or CD19-co-CD20 CAR-T) or non-transduced (e.g., unT) T cells (1×10⁵/well) were co-cultured with Raji target cells (4×10⁵ well) at an E:T ratio of 1:4 in 24-well plates, in the absence of exogenous cytokines (e.g., IL-2). Part of the cells were harvested and stained for CD3 after 2 or 3 days of co-culture. CAR-T cells were identified by CD3 and CAR signal. For serial co-culture assays, the remaining T cells were then re-challenged with fresh Raji target cells at the same E:T ratio. Co-cultures were carried on until the tumor cells outgrew the well volume. The T cell proliferation rate at each time point was calculated by dividing the number of T cells at the time point by the initial number of T cells.

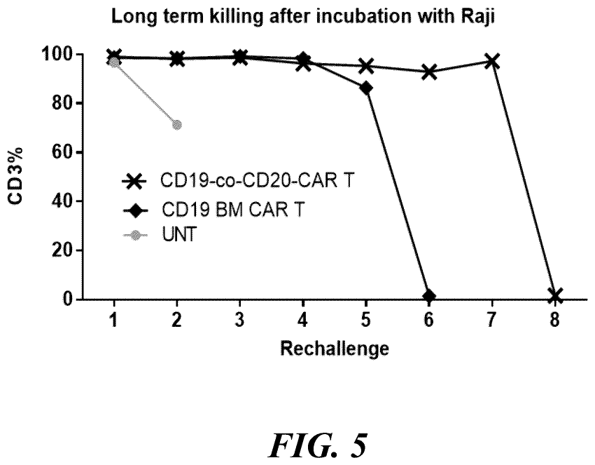

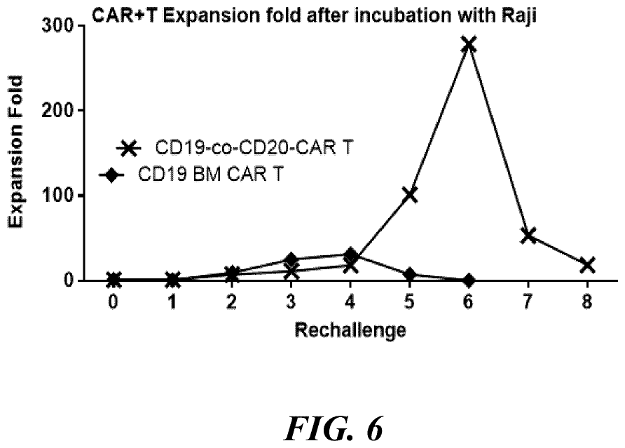

The killing efficacy of various CAR-T cells in the repeated tumor stimulation assay is shown in FIG. 5. The CD19 BM CAR-T cells were exhausted after 5 rounds of tumor stimulation (e.g., could not kill additional target tumor cells, as measured by CD3%), whereas CD19-co-CD20 CAR-T cells persisted killing target tumor cells to 7 rounds of tumor stimulation. In addition, CD19-co-CD20 CAR-T cells proliferated and exhibited better long-term expansion than CD19 BM CAR-T cells in vitro (FIG. 6). These results demonstrate that TLR4 signaling activation renders CAR-T cells resistant to exhaustion and enhances their ability to kill target tumor cells in vitro, thus addressing major barriers to progress with this class of therapeutic agents.

### Example 3: Preparation of CAR-T Cells Expressing Exogenously Introduced CLL1 and CD33 Toll-Like Receptor (TLR) Domains

This example shows the construction of exemplary armored CAR-T cells expressing exogenously introduced toll-like receptor (TLR) domains. In particular, this example shows the construction of CLL1 and/or CD33 expressing CAR-T cells.

### 3.1. Construction of Chimeric Antigen Receptors (CARs)

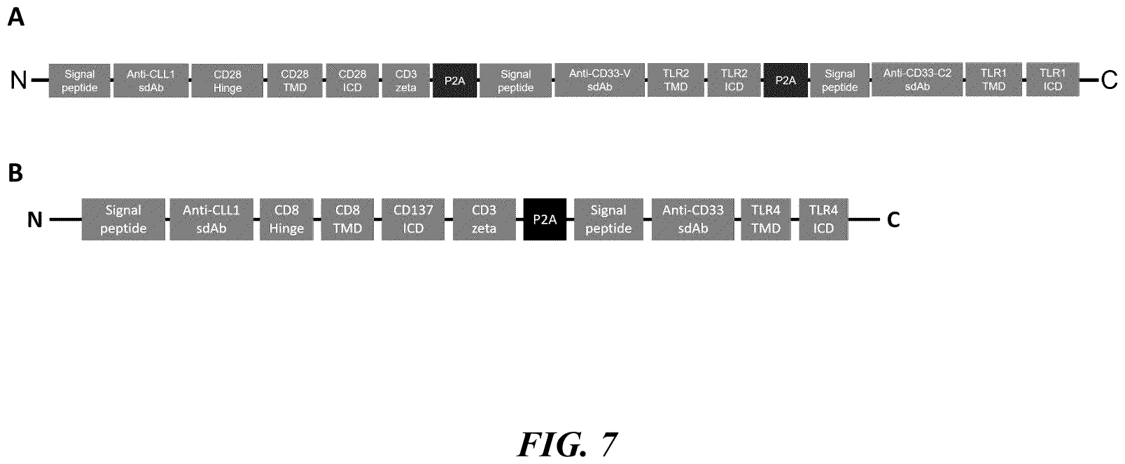

This example contains two CAR constructs. The first CAR construct named CLL1-co-CD33 CAR (SEQ ID NO: 3), a CAR backbone sequence encoding a CAR backbone polypeptide comprising from the N-terminus to the C-terminus: a signal peptide (SEQ ID NO: 5), anti-CLL1 sdAb (SEQ ID NO: 14), a CD28a hinge domain (SEQ ID NO: 15), a CD28 transmembrane (TM) domain (SEQ ID NO: 16), the cytoplasmic portion of the CD28 molecule (SEQ ID NO: 17), and a CD3ζ primary intracellular signaling domain (SEQ ID NO: 10), was codon optimized and chemically synthesized. The sequence of a first inducible costimulatory molecule comprising from the N-terminus to the C-terminus: a signal peptide (SEQ ID NO: 5), an anti-CD33 V domain sdAb (SEQ ID NO: 21), a TLR2 transmembrane (TM) region (SEQ ID NO: 22), and the cytoplasmic portion of TLR2 (SEQ ID NO: 23), was also codon optimized and chemically synthesized, and linked to the CAR backbone sequence via a P2A cleavage site (SEQ ID NO: 24). The sequence of a second inducible costimulatory molecule comprising from the N-terminus to the C-terminus: a signal peptide (SEQ ID NO: 5), an anti-CD33 C2 domain sdAb (SEQ ID NO: 18), a TLR1 transmembrane (TM) region (SEQ ID NO: 19), and the cytoplasmic portion of TLR1 (SEQ ID NO: 20), was also codon optimized and chemically synthesized, and linked to the first inducible costimulatory molecule via a P2A cleavage site (SEQ ID NO: 24). This construct is shown in FIG. 7.

The second CAR construct named CLL1-co-CD33-2 CAR (SEQ ID NO: 71), a CAR backbone sequence encoding a CAR backbone polypeptide comprising from the N-terminus to the C-terminus: a signal peptide (SEQ ID NO: 5), anti-CLL1 sdAb (SEQ ID NO: 14), a CD8α hinge domain (SEQ ID NO: 7), a CD8α transmembrane (TM) domain (SEQ ID NO: 8), the cytoplasmic portion of the 4-1BB(CD137) molecule (SEQ ID NO: 9), and a CD3ζ primary intracellular signaling domain (SEQ ID NO: 10), was codon optimized and chemically synthesized. The sequence of inducible costimulatory molecule comprising from the N-terminus to the C-terminus: a signal peptide (SEQ ID NO: 5), an anti-CD33 sdAb (SEQ ID NO: 21), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24). This construct is shown in FIG. 7.

The CAR backbone and costimulatory molecule were cloned into a lentiviral vector (e.g., a second generation lentiviral vector), downstream and operably linked to a constitutive hEF1α promoter for in vitro transcription. Transient retroviral supernatants were produced refer as exemplified in Blood (2006) 108 (12): 3890-3897.

A construct encoding anti-CLL1 benchmark CAR (CLL1 BM CAR) was also prepared as described above for comparative analysis (SEQ ID NO: 4).

A construct encoding anti-CLL1/CD33 dual CAR (CLL1/CD33 dual CAR) was also prepared as described above for comparative analysis (SEQ ID NO: 43).

### 3.2. Viral Preparation and Transduction of T Cells

Lentiviruses were prepared as described in Example 1. T cell lymphocytes were collected and transduced with the lentiviruses according to the methods described in Example 1.

### Example 4: In Vitro Cytotoxicity Assay of CLL1-Co-CD33 CAR-T Cells

This example shows the in vitro anti-tumor activity of CLL1-co-CD33 CAR-T and CLL1-co-CD33-2 CAR-T cells.

### 4.1 Lactate Dehydrogenase (LDH) Cytoxicity

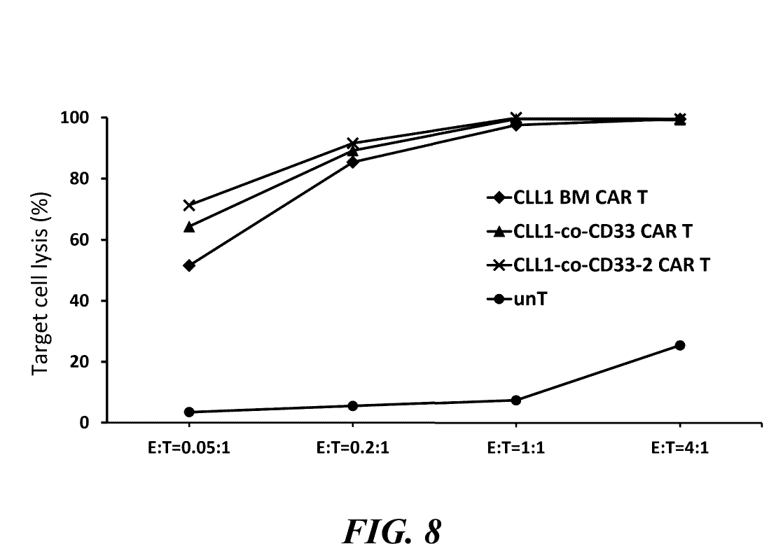

LDH assays were performed to measure cytotoxicity following 24 h of culture, as described in Example 2.1. CLL1 and CD33-expressing AML cell line U937 was used as the target cells. As shown in FIG. 8, CLL1 BM CAR-T cells, CLL1-co-CD33 CAR-T and CLL1-co-CD33-2 CAR-T cells showed comparable in vitro cytotoxicity against U937 target cells.

### 4.2 Cytokine Secretion by Homogeneous Time Resolved Fluorescence (HTRF)

Supernatants from the in vitro cytotoxicity assay were collected to assess CAR-induced cytokine release, and Homogeneous Time Resolved Fluorescence (HTRF) assays for IFN-γ (Cisbio) were performed according to the manufacturer's manual and Example 2.2.

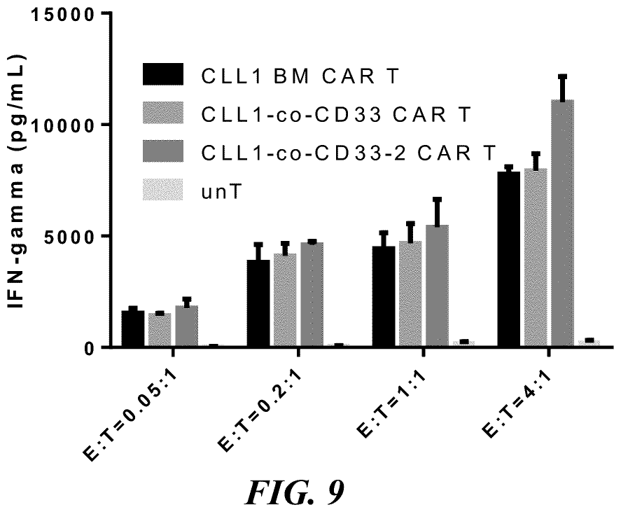

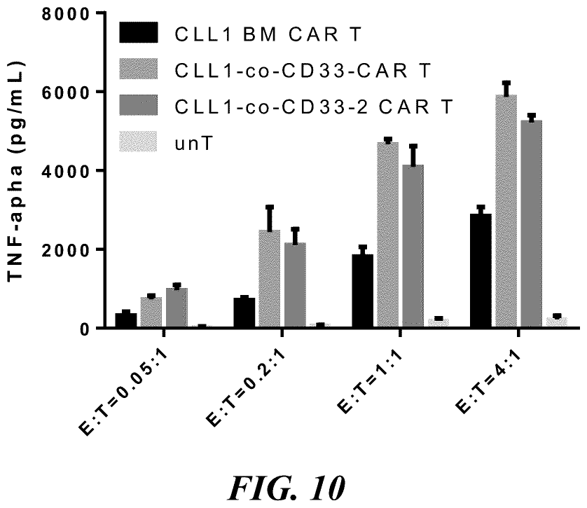

CAR-T cells (e.g., CLL1 BM CAR-T cells, CLL1-co-CD33 CAR-T and CLL1-co-CD33-2 CAR-T and untransduced “unT” CAR-T cells) were co-cultured with U937 target cells. The culture supernatants were collected after 24 h to assess IFNγ and TNFα release as a measure of T cell activation. As shown in FIGS. 9 and 10, CLL1-co-CD33 CAR-T and CLL1-co-CD33-2 CAR-T cells co-cultured with target cells secreted significant amounts of TNFα and IFNγ. Moreover, the level of both TNFα and IFNγ secretion was higher than that of positive control CLL1 BM CAR-T cells.

### 4.3 Long-Term Co-Culture Assay

To evaluate the long-term killing efficacy of CLL1-co-CD33 CAR-T cells, long-term co-culture assays were performed to mimic the dynamic killing process in vivo. Transduced (e.g., CLL1 BM CAR-T, CLL1-co-CD33 CAR-T or CLL1-co-CD33-2 CAR-T) or non-transduced (e.g., unT) T cells (1×10⁵/well) were co-cultured with U937 target cells (4×10⁵ well) at an E:T ratio of 1:4 in 24-well plates, in the absence of exogenous cytokines (e.g., IL-2). Part of the cells were harvested and stained for CD3 after 2 or 3 days of co-culture. CAR-T cells were identified by CD3 and CAR signal. For serial co-culture assays, the remaining T cells were then re-challenged with fresh U937 target cells at the same E:T ratio. Co-cultures were carried on until the tumor cells outgrew the well volume. The T cell proliferation rate at each time point was calculated by dividing the number of T cells at the time point by the initial number of T cells.

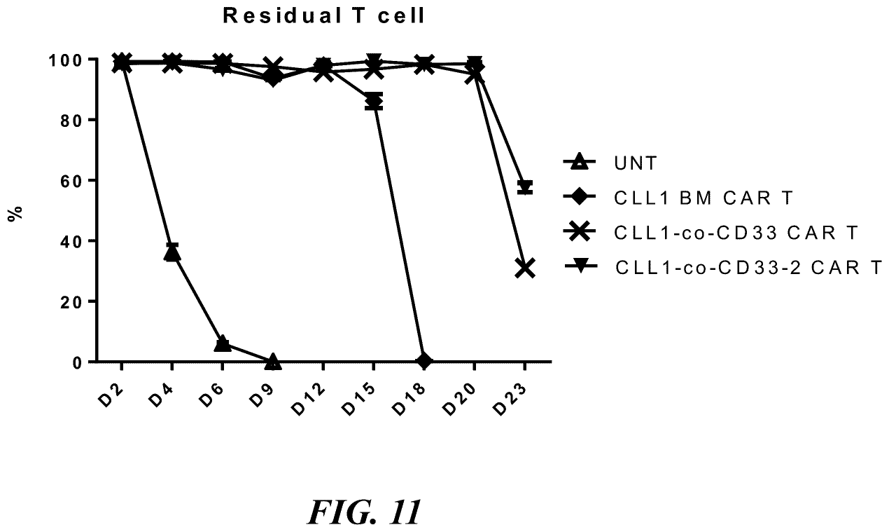

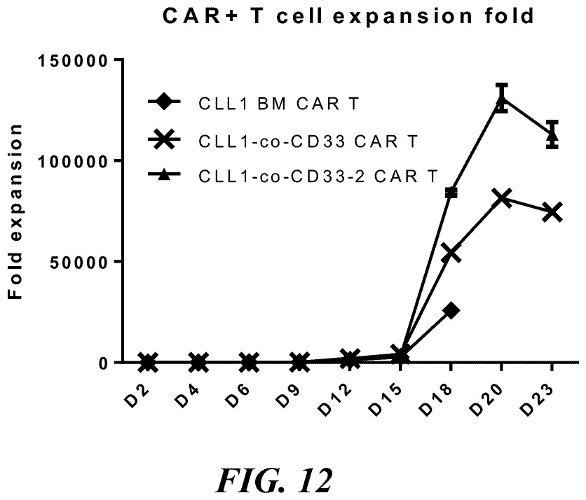

The killing efficacy of various CAR-T cells in the repeated tumor stimulation assay is shown in FIG. 11. The CLL1 BM CAR-T cells were unable to kill target tumor cells after day 15 of re-challenge, whereas CLL1-co-CD33 CAR-T and CLL1-co-CD33-2 CAR-T cells persisted killing target tumor cells to day 20 of tumor stimulation. In addition, CLL1-co-CD33 CAR-T and CLL1-co-CD33-2 CAR-T cells proliferated and exhibited better long-term expansion than CLL1 BM CAR-T cells in vitro (FIG. 12). These results demonstrate that the chimeric TLR can provide a potent costimulatory signal that enhances T cell survival and augments T cell proliferation in the context of CAR signaling.

### Example 5: Evaluation of CLL1-Co-CD33 CAR-T In Vivo Mouse Model

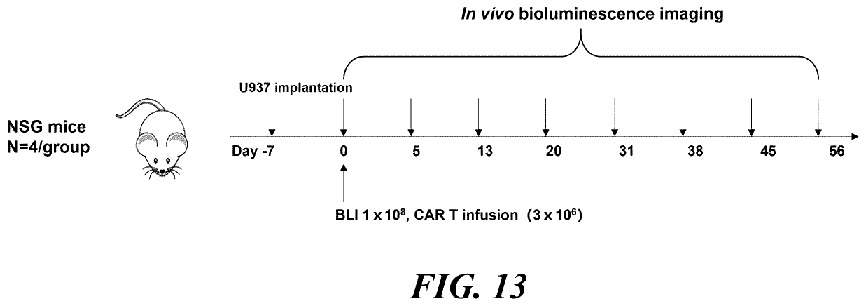

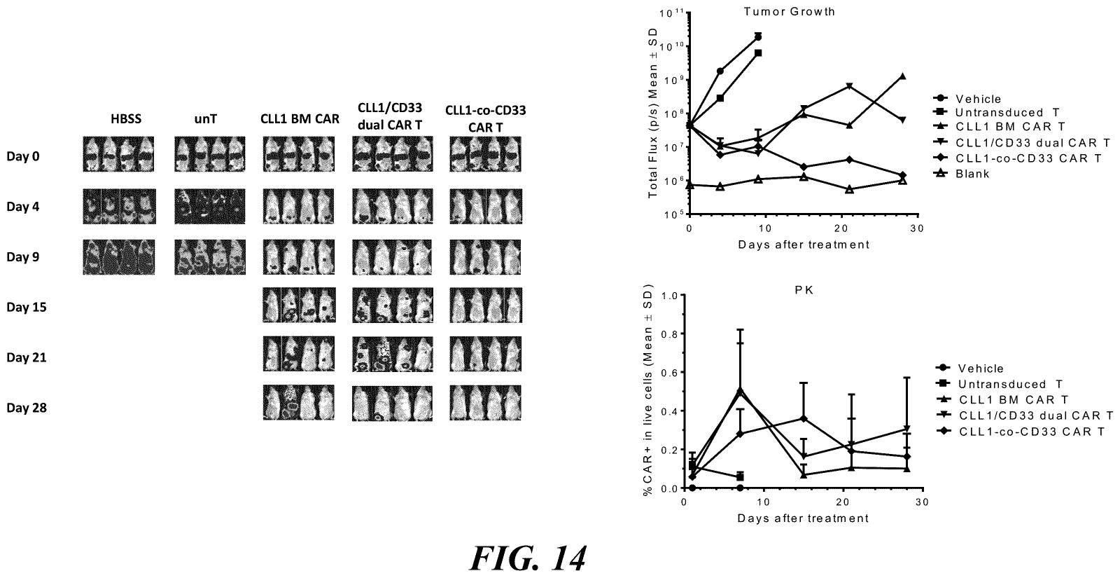

Anti-tumor activities of armored TLR CAR-T cells were assessed in vivo in a U937-Luc xenograft mouse model according to the schedule shown in FIG. 13. 2×10⁶ U937-Luc cells with the firefly luciferase reporter gene expression were implanted subcutaneously on day 0 in NOD/SCID IL-2RγCnull (NSG) mice. Bioluminescent imaging (BLI) was conducted weekly or biweekly post tumor inoculation to monitor model development. The animals were randomized based on the BLI photon numbers and animal body weights. After randomization, a single dose of CAR-T cells or UnT cells were infused intravenously. Weekly BLI imaging was performed to record tumor growth. Bioluminescence imaging (BLI) showed that a reduced tumor burden was present in mice infused with TLR armored CAR T cells compared with CLL1 BM CAR T and CLL1/CD33 dual CAR T cells (FIG. 14). Consistently, the percentages of CAR T cells in the peripheral blood mononuclear cells (PBMC) were significantly higher in mice treated with TLR armored CAR T cells compared with CLL1 BM CAR and CLL1/CD33 dual CAR T cells (FIG. 14). Thus, CLL1-co-CD33 CAR-T were more potent than CLL1 BM or CLL1/CD33 dual CAR T cells at killing U937-GL cells in vivo.

### Example 6: Evaluation of CLL1-Co-CD33 CAR-γδ T

Gamma delta T (γδT) lymphocytes develop mainly in the thymus and generate their 76 T cell receptor through V(D)J recombination, and are primed for rapid function, including cytotoxicity toward cancer cells. Another potential advantage of γδ T cells is that, unlike αβ T cells, they are not restricted to MHC, and thus utilizing engineered allogeneic donor derived γδ T cells expressing CAR transgene could theoretically be used as an off-the-shelf universal product, though this application would be limited to very immunocompromised patients. Therefore, we also assessed the efficacy of TLR armored CAR (e.g., CLL1-co-CD33 and CLL1-co-CD33-2) modified γδ T.

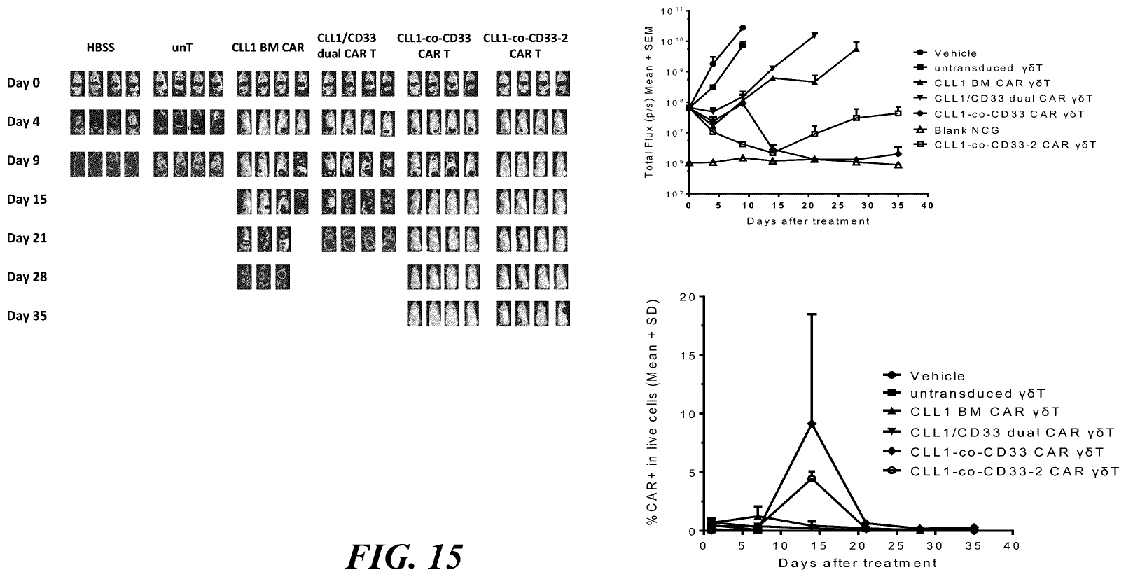

The in vivo efficacy of CLL1-co-CD33 CAR γδ T cells were evaluated in a U937-Luc xenograft mouse model as described in Example 5. As shown in FIG. 15, mice treated with CLL1-co-CD33 CAR γδ T showed persistent U937 tumor clearance in all mice. However, both CLL1 BM CAR and CLL1/CD33 dual CAR γδ T group experienced tumor relapse. Consistently, the percentages of CAR γδ T cells in the peripheral blood mononuclear cells (PBMC) were significantly higher in mice treated with CLL1-co-CD33 or CLL1-co-CD33-2 CAR γδ T cells compared with CLL1 BM CAR and CLL1/CD33 dual CAR γδ T cells.

### Example 7: Preparation of CAR γδ T Cells Expressing Exogenously Introduced GPC3 and NKG2D CD8 TLR4 Chimeric Receptors

This example shows the construction of exemplary armored CAR γδ T cells expressing exogenously introduced NKG2D CD8 TLR4 chimeric receptors. In particular, this example shows the construction of GPC3 expressing CAR γδ T cells.

### 7.1. Construction of Chimeric Antigen Receptors (CARs)

To construct an anti-GPC3 armored NKG2D TLR4 chimeric receptors (SEQ ID NO: 57), a CAR backbone sequence encoding a CAR backbone polypeptide comprising from the N-terminus to the C-terminus: a signal peptide (SEQ ID NO: 5), anti-GPC3 scFv (SEQ ID NO: 55), a CD8α hinge domain (SEQ ID NO: 7), a CD8 transmembrane (TM) domain (SEQ ID NO: 8), the cytoplasmic portion of the 4-1BB molecule (SEQ ID NO: 9), and a CD3ζ primary intracellular signaling domain (SEQ ID NO: 10), was codon optimized and chemically synthesized. The sequence of the NKG2D TLR4 chimeric receptors comprising from the N-terminus to the C-terminus: a signal peptide (SEQ ID NO: 5), an NKG2D ECD (SEQ ID NO: 51), a TLR4 transmembrane (TM) region (SEQ ID NO: 12), and the cytoplasmic portion of TLR4 (SEQ ID NO: 13), was codon optimized and chemically synthesized, and linked to the CAR backbone sequence via a P2A cleavage site (SEQ ID NO: 24).

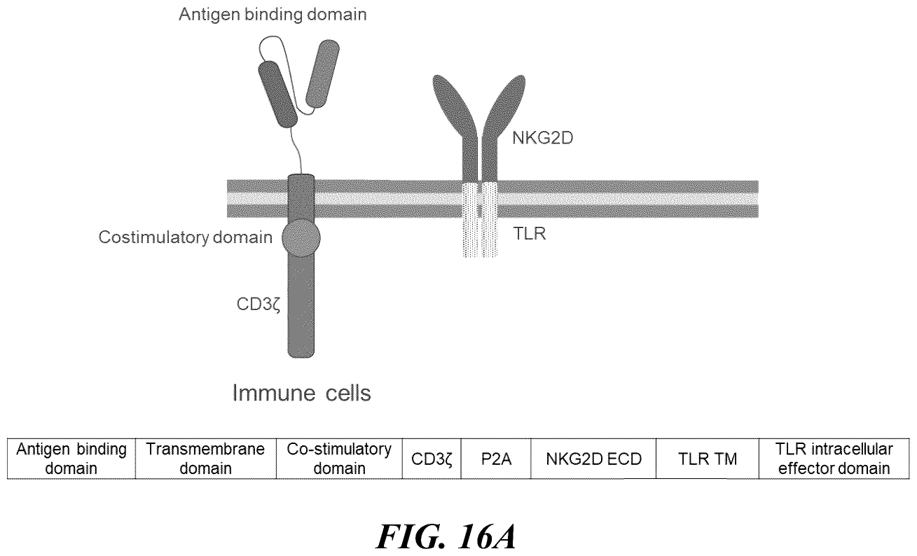

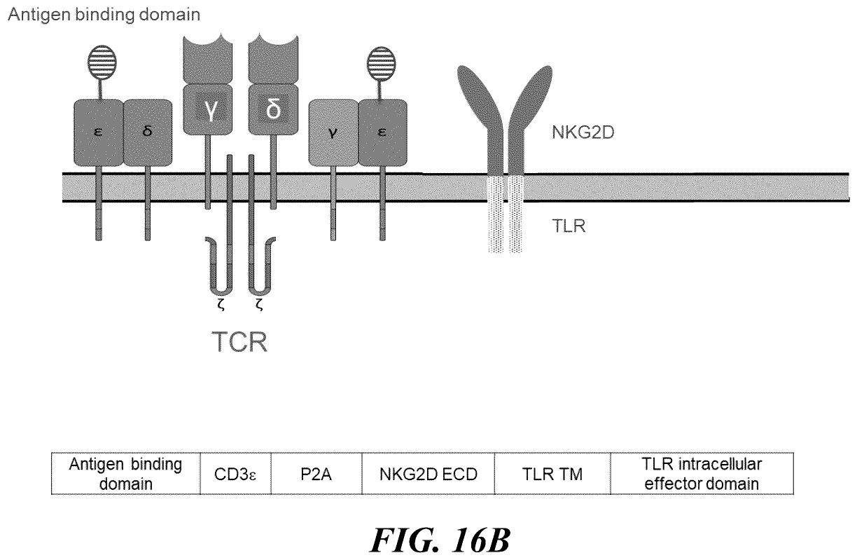

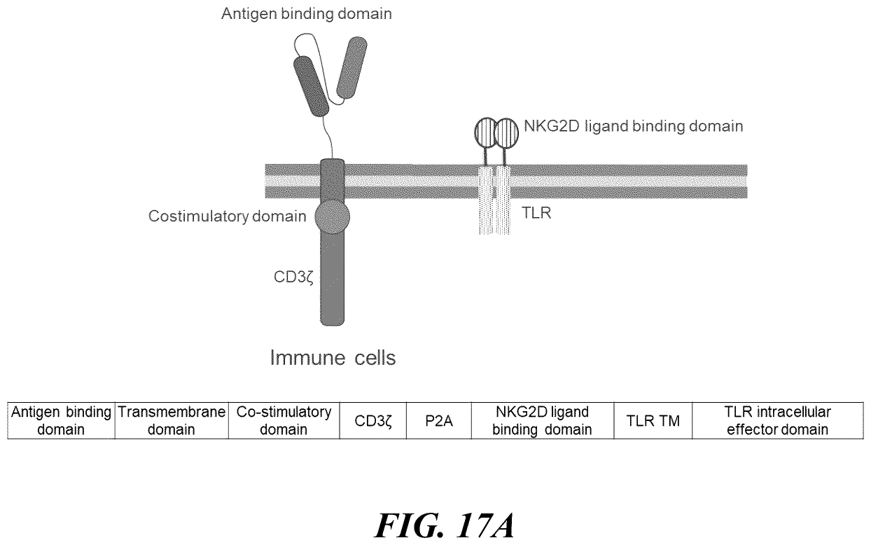

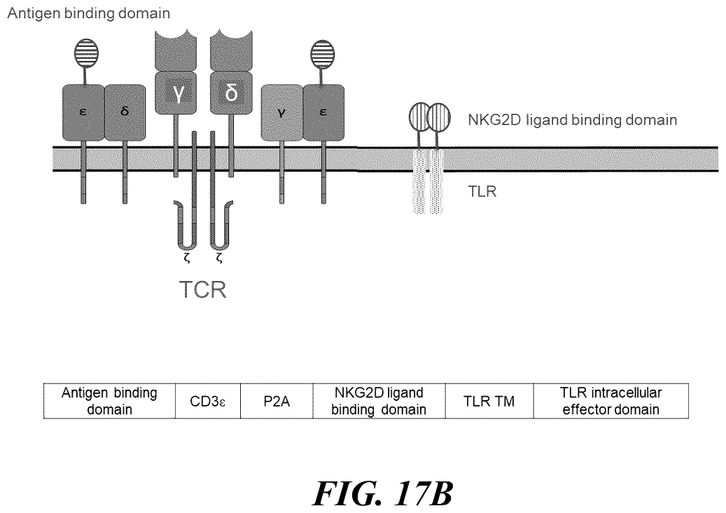

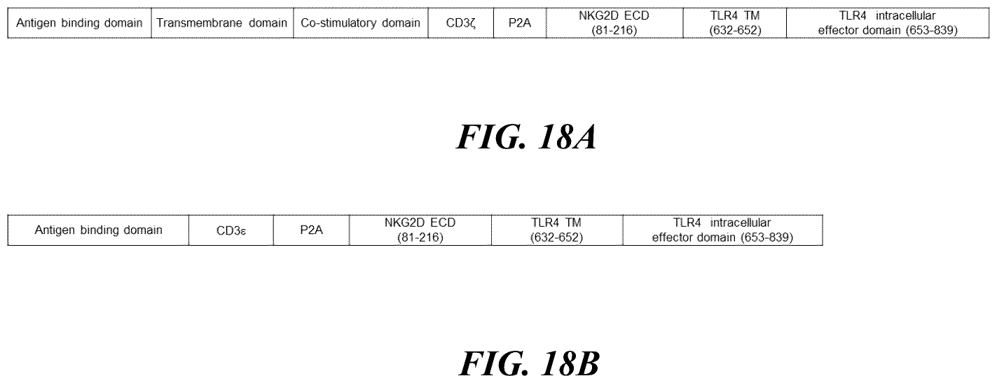

To construct an anti-GPC3 armored NKG2D CD8 TLR4 chimeric receptors (SEQ ID NO: 58), a CAR backbone sequence encoding a CAR backbone polypeptide comprising from the N-terminus to the C-terminus: a signal peptide (SEQ ID NO: 5), anti-GPC3 scFv (SEQ ID NO: 55), a CD8α hinge domain (SEQ ID NO: 7), a CD8 transmembrane (TM) domain (SEQ ID NO: 8), the cytoplasmic portion of the 4-1BB molecule (SEQ ID NO: 9), and a CD3ζ primary intracellular signaling domain (SEQ ID NO: 10), was codon optimized and chemically synthesized. The sequence of the NKG2D CD8 TLR4 chimeric receptors comprising from the N-terminus to the C-terminus: a signal peptide (SEQ ID NO: 5), an NKG2D ECD (SEQ ID NO: 51), a CD8α hinge (SEQ ID NO: 7), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24). This construct is shown in FIG. 16A.

The CAR backbone and NKG2D TLR4/NKG2D CD8 TLR4 chimeric receptors were cloned into a lentiviral vector (e.g., a second generation lentiviral vector), downstream and operably linked to a constitutive hEF1α promoter for in vitro transcription. Transient retroviral supernatants were produced refer as exemplified in Blood (2006) 108 (12): 3890-3897.

A construct encoding anti-GPC3 CAR (GPC3 CAR) was also prepared as described above for comparative analysis (SEQ ID NO: 56).

### 7.2. Viral Preparation

Lentiviruses were prepared as described in Example 1.

### 7.3. Transduction of γδ T Cells

γδ T cells were prepared by addition of 5 μM Zoledronate and 1000 IU/mL IL-2 to PBMCs and cultured for 9 days with periodical change of media supplemented with 1000 IU/mL IL-2. Alternatively, γδ T cells were isolated from PBMC or umbilical cord blood (UCB) and then stimulated by anti-γδ TCR antibody and anti-CD3 (OKT3) followed by co-incubation of K562-based artificial antigen-presenting cells (aAPCs) at an 1:2 ratio for at least 10 days. PBMCs were isolated by density centrifugation (lymphoprep) from leukapheresis material and cryopreserved. PBMCs were resuscitated and activated with zoledronic acid (5 μM) in cell culture media AIM-V supplemented with IL-2 (1000 IU/ml) and 5% human AB serum and kept in a humidified chamber (37° C., 5% CO₂). 48 hours post-activation, cells were transduced with lentiviral vectors encoding the system of Example 1 at an MOI of 5 with 5 μg/ml polybrene. Such transduction procedure was repeated the next day followed by replenishment of fresh media containing IL-2 (1000 IU/ml) the second day after the transduction. Cells were cultured in AIM-V supplemented with IL-2 (1000 IU/ml) in a humidified chamber with periodical change of media as determined by the pH of the culture media for further expansion. Cells were harvested 10 days post-transduction and the total number, purity and transduction efficiency were determined. Cells were further enriched with a negative TCRγ/δ⁺ T cell isolation kit (Miltenyi Biotec) before future applications or cryopreserved.

### Example 8: In Vitro Cytotoxicity Assay of Anti-GPC3 CAR γδ T Cells Armored with NKG2D CD8 TLR4 Chimeric Receptors

This example shows the in vitro anti-tumor activity of anti-GPC3 CAR γδ T cells armored with NKG2D CD8 TLR4 chimeric receptors.

### 8.1 Long-Term Co-Culture Assay

To evaluate the long-term killing efficacy of CAR γδ T cells, we performed long-term co-culture assays, which mimic the dynamic killing process in vivo. Transduced or non-transduced T cells (1×10⁵/well) were co-cultured with tumor cell lines (Huh7 cells, 1×10⁵/well) at an E:T ratio of 1:1 in 24-well plates, in the absence of exogenous cytokines (IL-2). Part of the cells were harvested and stained for CD3 after 2 or 3 days' co-culture. For serial co-culture assays, the remaining T cells were then re-challenged with fresh Huh7 cells at the same E:T ratio. Co-cultures were carried on until tumor cells outgrew. The T cell proliferation rate at each time point was calculated by dividing the number of T cells at the time point by the number of T cells at a previous time point.

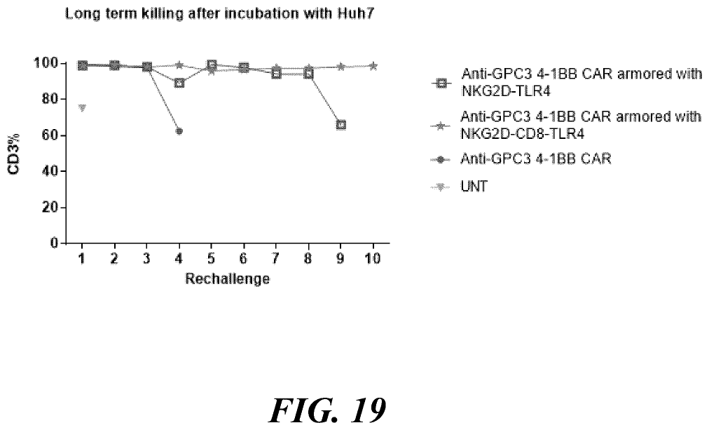

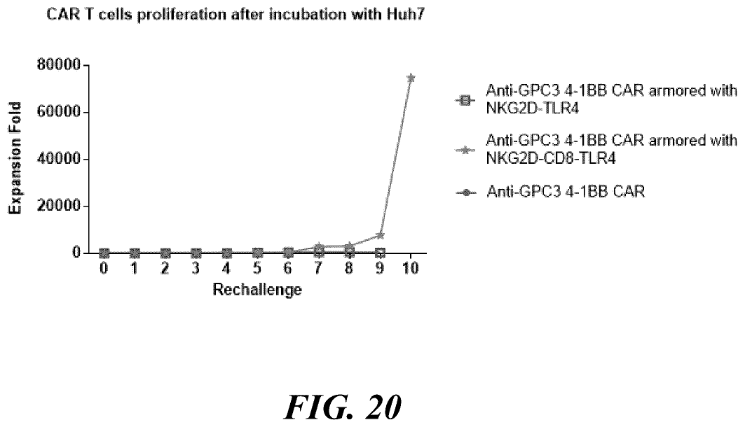

Representative result of long-term co-culture assay by FACS detection is shown in FIG. 19. Calculated T cell proliferation from the same experiment is shown in FIG. 20. The data indicated that NKG2D CD8 TLR4 chimeric receptors armored anti-GPC3 CAR γδ T cells have better cytotoxicity and proliferation than anti-GPC3 CAR γδ T cells in vitro.

### 8.2 IFN-γ Secretion Detected by HTRF

Another measure of effector T-cell activation and proliferation is the production of effector cytokines such as IFN-γ. Supernatants from the round1 to round 3 in vitro cytotoxicity assay were collected to assess CAR-induced cytokine release. HTRF assays for IFN-γ (Cisbio, Cat #62HIFNGPEH) was performed according to the manufacturer's manual.

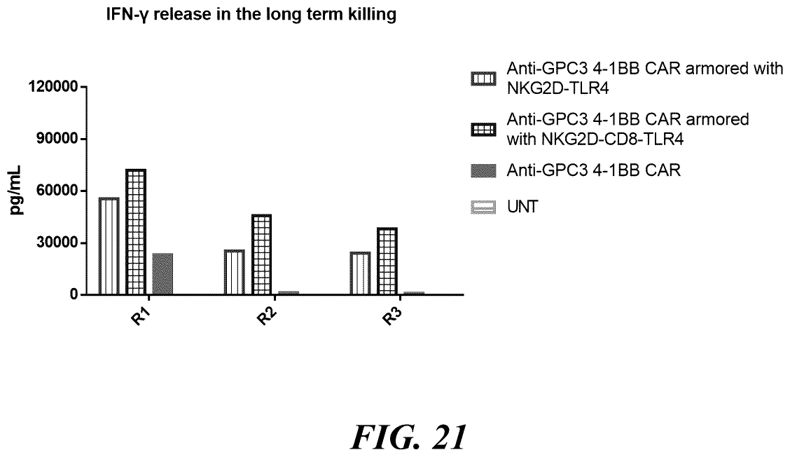

The corresponding cytokine release result is shown in FIG. 21. Both anti-GPC3 γδ CAR T cells exhibited potent killing activity against Huh7 cells, and released IFN-γ in response to Huh7 cells. CAR T cells armored with NKG2D CD8 TLR4 chimeric receptor showed higher level of IFN-γ secretion than naked CAR T cells.

### Example 9: Preparation of CAR Up T Cells Expressing Exogenously Introduced CD19 and NKG2D CD8 TLR4 Chimeric Receptors

This example shows the construction of exemplary armored CAR αβ T cells expressing exogenously introduced NKG2D CD8 TLR4 chimeric receptors. In particular, this example shows the construction of CD19 expressing CAR αβ T cells.

### 9.1. Construction of Chimeric Antigen Receptors (CARs)

To construct an anti-CD19 armored NKG2D TLR4 chimeric receptors (SEQ ID NO: 59), a CAR backbone sequence encoding a CAR backbone polypeptide comprising from the N-terminus to the C-terminus: a signal peptide (SEQ ID NO: 5), anti-CD19 scFv (SEQ ID NO: 6), a CD8α hinge domain (SEQ ID NO: 7), a CD8 transmembrane (TM) domain (SEQ ID NO: 8), the cytoplasmic portion of the 4-1BB molecule (SEQ ID NO: 9), and a CD3ζ primary intracellular signaling domain (SEQ ID NO: 10), was codon optimized and chemically synthesized. The sequence of the NKG2D TLR4 chimeric receptors comprising from the N-terminus to the C-terminus: a signal peptide (SEQ ID NO: 5), an NKG2D ECD (SEQ ID NO: 51), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24).

To construct an anti-CD19 armored NKG2D CD8 TLR4 chimeric receptors (SEQ ID NO: 60), a CAR backbone sequence encoding a CAR backbone polypeptide comprising from the N-terminus to the C-terminus: a signal peptide (SEQ ID NO: 5), anti-CD19 scFv (SEQ ID NO: 6), a CD8α hinge domain (SEQ ID NO: 7), a CD8 transmembrane (TM) domain (SEQ ID NO: 8), the cytoplasmic portion of the 4-1BB molecule (SEQ ID NO: 9), and a CD3ζ primary intracellular signaling domain (SEQ ID NO: 10), was codon optimized and chemically synthesized. The sequence of the NKG2D CD8 TLR4 chimeric receptors comprising from the N-terminus to the C-terminus: a signal peptide (SEQ ID NO: 5), an NKG2D ECD (SEQ ID NO: 51), a CD8α hinge (SEQ ID NO: 7), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24). This construct is shown in FIG. 16A.

The CAR backbone and NKG2D TLR4/NKG2D CD8 TLR4 chimeric receptors were cloned into a lentiviral vector (e.g., a second generation lentiviral vector), downstream and operably linked to a constitutive hEF1α promoter for in vitro transcription. Transient retroviral supernatants were produced refer as exemplified in Blood (2006) 108 (12): 3890-3897.

A construct encoding anti-CD19 CAR (CD19 BM CAR) was also prepared as described above for comparative analysis (SEQ ID NO: 2).

### 9.2. Viral Preparation and Transduction of T Cells

Lentiviruses were prepared as described in Example 1. T cell lymphocytes were collected and transduced with the lentiviruses according to the methods described in Example 1.

### Example 10: In Vitro Cytotoxicity Assay of Anti-CD19 CAR Up T Cells Armored with NKG2D CD8 TLR4 Chimeric Receptors

This example shows the in vitro anti-tumor activity of anti-CD19 CAR αβ T cells armored with NKG2D CD8 TLR4 chimeric receptors.

### Long-Term Co-Culture Assay

To evaluate the long-term killing efficacy of CAR αβ T cells, we performed long-term co-culture assays. Transduced or non-transduced T cells (1×10⁵/well) were co-cultured with tumor cell lines (Raji cells, 1×10⁵/well) at an E:T ratio of 1:1 in 24-well plates, in the absence of exogenous cytokines (IL-2). Part of the cells were harvested and stained for CD3 after 2 or 3 days' co-culture.

For serial co-culture assays, the remaining T cells were then re-challenged with fresh Raji cells at the same E:T ratio. Co-cultures were carried on until tumor cells outgrew. The T cell proliferation rate at each time point was calculated by dividing the number of T cells at the time point by the number of T cells at a previous time point.

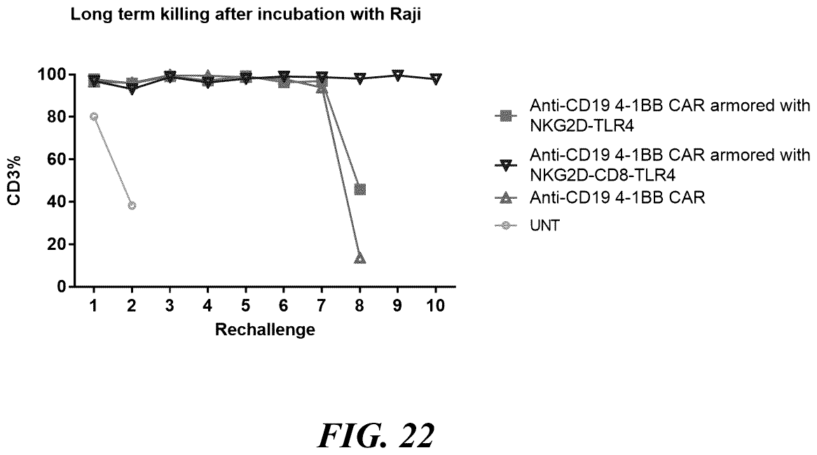

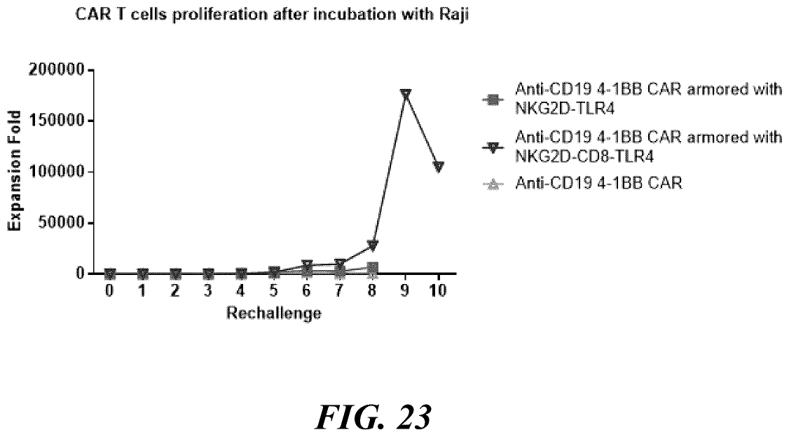

Representative result of long-term co-culture assay by FACS detection was shown in FIG. 22. Calculated T cell proliferation from the same experiment were shown in FIG. 23. The data indicated that NKG2D CD8 TLR4 chimeric receptor armor improved anti-CD19 CAR αβ T cells cytotoxicity and proliferation.

### Example 11: In Vivo Efficacy Evaluation of Anti-GPC3 CAR γδ T Cells Armored with NKG2D CD8 TLR4 Chimeric Receptors

This example shows the in vivo anti-tumor activity of anti-GPC3 CAR γδ T cells armored with NKG2D CD8 TLR4 chimeric receptors.

Anti-tumor activity of an exemplary anti-GPC3 CAR-γδ T cells was assessed in vivo in a Huh7 xenograft model. Briefly, 3 million (3×10⁶) Huh7 cells were implanted subcutaneously on day 0 in NOD/SCID IL-2RγC null (NSG) mice. Ten days after tumor inoculation, mice were treated with intravenous injection of 1×10⁶ armored CAR-γδ T or mock T cells or phosphate-buffered saline (PBS). Tumor dimensions were measured with calipers twice a week, and tumor volumes were calculated using the formula V=1/2 (length×width²). Mice were euthanized when the mean tumor burden in the control mice reached 2,000 mm³.

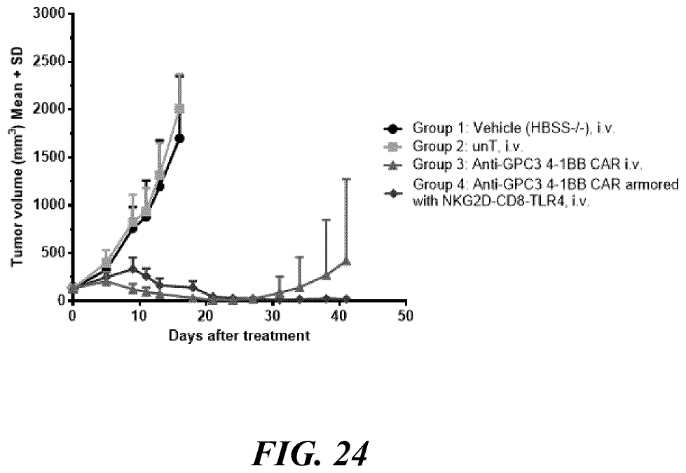

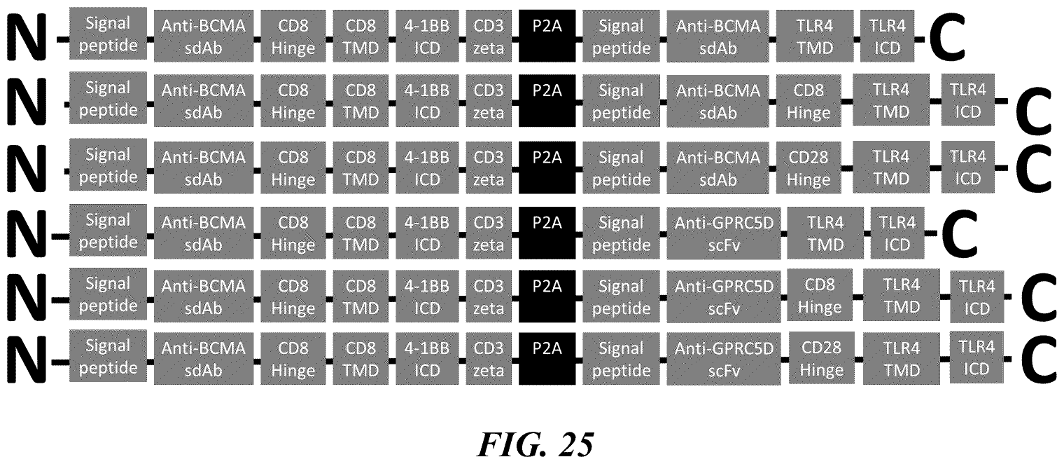

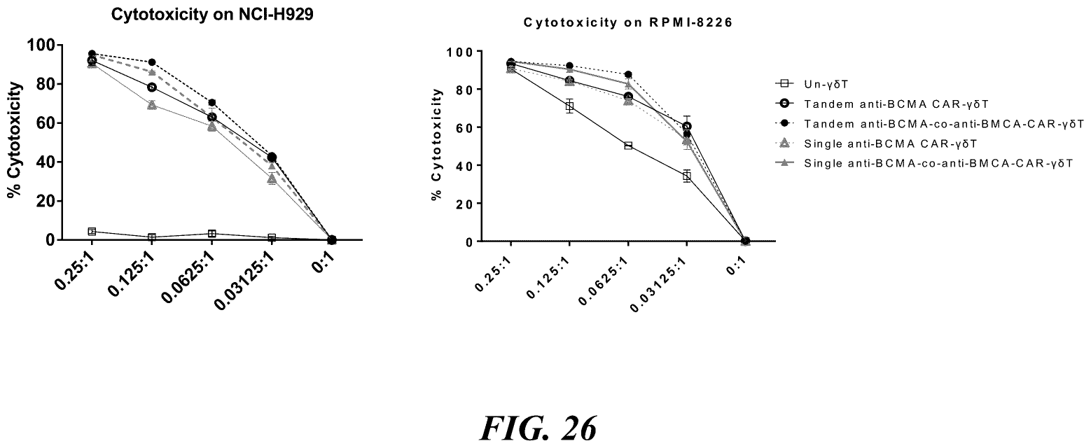

The results of anti-tumor effect of anti-GPC3 CAR γδT cells or anti-GPC3 CAR armored with NKG2D CD8 TLR4 chimeric receptors γδT cells in Huh7 xenograft model were shown in FIG. 24. Unarmored CAR-γδ T cells, alongside NKG2D CD8 TLR4 chimeric receptors armored CAR-γδ T cells inhibited tumor growth. Specifically, unarmored CAR-γδ T cells-treated mice reached tumor free but slowly repulsed, while NKG2D CD8 TLR4 chimeric receptors armored CAR-γδ T cells-treated mice reached tumor-free and remained healthy and tumor-free till the end of experimental observations.

### Example 12: Preparation of CAR-T Cells Expressing Exogenously Introduced Toll-Like Receptor 4 (TLR4) Intracellular Domains with Tandem, Single Anti-BCMA sdAb or Anti-GPRC5D scFv

This example shows the construction of exemplary armored CAR-T cells expressing exogenously introduced toll-like receptor (TLR) domains. In particular, this example shows the construction of anti-BCMA CAR-T cells with tandem, single sdAbs or anti-GPRC5D scFv.

### 12.1. Construction of Chimeric Antigen Receptors (CARs)

To construct a tandem anti-BCMA-co-anti-BCMA CAR (SEQ ID NO: 61), a CAR backbone sequence encoding a CAR backbone polypeptide comprising from the N-terminus to the C-terminus: a signal peptide (SEQ ID NO: 5), tandem anti-BCMA sdAb (SEQ ID NO: 44), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), was codon optimized and chemically synthesized. The sequence of an inducible costimulatory molecule comprising from the N-terminus to the C-terminus: a signal peptide (SEQ ID NO: 5), tandem anti-BCMA sdAb (SEQ ID NO: 44), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24).

To construct a single anti-BCMA-co-anti-BCMA CAR (SEQ ID NO: 62), a CAR backbone sequence encoding a CAR backbone polypeptide comprising from the N-terminus to the C-terminus: a signal peptide (SEQ ID NO: 5), a anti-BCMA sdAb (SEQ ID NO: 68), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), was codon optimized and chemically synthesized. The sequence of an inducible costimulatory molecule comprising from the N-terminus to the C-terminus: a signal peptide (SEQ ID NO: 5), anti-BCMA sdAb (SEQ ID NO: 69), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24).

To construct a single anti-BCMA-co-anti-BCMA-CD8 CAR (SEQ ID NO: 63), a CAR backbone sequence encoding a CAR backbone polypeptide comprising from the N-terminus to the C-terminus: a signal peptide (SEQ ID NO: 5), a anti-BCMA sdAb (SEQ ID NO: 68), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), was codon optimized and chemically synthesized. The sequence of an inducible costimulatory molecule comprising from the N-terminus to the C-terminus: a signal peptide (SEQ ID NO: 5), anti-BCMA sdAb (SEQ ID NO: 69), a CD8α hinge domain (SEQ ID NO: 7), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24).

To construct a single anti-BCMA-co-anti-BCMA-CD28 CAR (SEQ ID NO: 64), a CAR backbone sequence encoding a CAR backbone polypeptide comprising from the N-terminus to the C-terminus: a signal peptide (SEQ ID NO: 5), a anti-BCMA sdAb (SEQ ID NO: 68), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), was codon optimized and chemically synthesized. The sequence of an inducible costimulatory molecule comprising from the N-terminus to the C-terminus: a signal peptide (SEQ ID NO: 5), anti-BCMA sdAb (SEQ ID NO: 69), a CD28 hinge domain (SEQ ID NO: 15), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24).

To construct a tandem anti-BCMA-co-anti-GPRC5D CAR (SEQ ID NO: 65), a CAR backbone sequence encoding a CAR backbone polypeptide comprising from the N-terminus to the C-terminus: a signal peptide (SEQ ID NO: 5), tandem anti-BCMA sdAb (SEQ ID NO: 44), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), was codon optimized and chemically synthesized. The sequence of an inducible costimulatory molecule comprising from the N-terminus to the C-terminus: a signal peptide (SEQ ID NO: 5), anti-GPRC5D scFv (SEQ ID NO: 70), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24).

To construct a tandem anti-BCMA-co-anti-GPRC5D-CD8 CAR (SEQ ID NO: 72), a CAR backbone sequence encoding a CAR backbone polypeptide comprising from the N-terminus to the C-terminus: a signal peptide (SEQ ID NO: 5), tandem anti-BCMA sdAb (SEQ ID NO: 44), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), was codon optimized and chemically synthesized. The sequence of an inducible costimulatory molecule comprising from the N-terminus to the C-terminus: a signal peptide (SEQ ID NO: 5), anti-GPRC5D scFv (SEQ ID NO: 70), a CD8α hinge domain (SEQ ID NO: 7), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24).

To construct a tandem anti-BCMA-co-anti-GPRC5D-CD28 CAR (SEQ ID NO: 73), a CAR backbone sequence encoding a CAR backbone polypeptide comprising from the N-terminus to the C-terminus: a signal peptide (SEQ ID NO: 5), tandem anti-BCMA sdAb (SEQ ID NO: 44), a CD8α hinge domain (SEQ ID NO: 7), a CD8α transmembrane (TM) domain (SEQ ID NO: 8), the cytoplasmic portion of the 4-1BB (CD137) co-stimulatory signaling domain (SEQ ID NO: 9), and a CD3ζ primary intracellular signaling domain (SEQ ID NO: 10), was codon optimized and chemically synthesized. The sequence of an inducible costimulatory molecule comprising from the N-terminus to the C-terminus: a signal peptide (SEQ ID NO: 5), anti-GPRC5D scFv (SEQ ID NO: 70), a CD28 hinge domain (SEQ ID NO: 15), a TLR4 transmembrane (TM) region (SEQ ID NO: 12), and the cytoplasmic portion of TLR4 (SEQ ID NO: 13), was also codon optimized and chemically synthesized, and linked to the CAR backbone sequence via a P2A cleavage site (SEQ ID NO: 24).

The CAR backbone and costimulatory molecule were cloned into a lentiviral vector (e.g., a second generation lentiviral vector), downstream and operably linked to a constitutive hEF1α promoter for in vitro transcription. Transient retroviral supernatants were produced refer as exemplified in Blood (2006) 108 (12): 3890-3897.

Separately, a tandem anti-BCMA CAR (SEQ ID NO: 66) and a single anti-BCMA CAR (SEQ ID NO: 67) were constructed comprising from the N-terminus to the C-terminus: a signal peptide (SEQ ID NO: 5), tandem anti-BCMA sdAb (SEQ ID NO: 44) or first single anti-BCMA sdAb (SEQ ID NO: 68), a CD8α hinge domain (SEQ ID NO: 7), a CD8α transmembrane (TM) domain (SEQ ID NO: 8), a 4-1BB co-stimulatory signaling domain (SEQ ID NO: 9), and a CD3ζ primary intracellular signaling domain (SEQ ID NO: 10), were prepared as described above and used for comparative analysis.

### 12.2. Construction of CAR-T Cells

### Viral Preparation

Lentiviruses were prepared as described in Example 1.

### Activation of γδT Cells

γδ T cells were prepared by addition of 5 μM Zoledronate and 1000 IU/mL IL-2 to PBMCs and cultured for 14 days with periodical change of media supplemented with 1000 IU/mL IL-2. Alternatively, γδ T cells were isolated from PBMC or umbilical cord blood (UCB) and then stimulated by anti-γδ TCR antibody and anti-CD3 (OKT3) followed by co-incubation of K562-based artificial antigen-presenting cells (aAPCs) at an 1:2 ratio for at least 10 days.

PBMCs were isolated by density centrifugation (lymphoprep) from leukapheresis material and cryopreserved. PBMCs were resuscitated and activated with zoledronic acid (5M) in cell culture media AIM-V supplemented with IL-2 (1000 IU/ml) and 5% human AB serum and kept in a humidified chamber (37° C., 5% C02). Forty-eight hours post-activation, cells were transduced with lentiviral vectors encoding the system of this example at an MOI of 2 with 5 μg/ml polybrene. Cells were cultured in AIM-V supplemented with IL-2 (1000 IU/ml) in a humidified chamber with periodical change of media as determined by the pH of the culture media for further expansion. Cells were harvested 10 days post-transduction and the total number, purity and transduction efficiency were determined. Cells were further enriched with a negative TCRγ/δ⁺ T cell isolation kit (Miltenyi Biotec) before future applications or cryopreserved.

### Virus Transduction of T Cells

Leukocytes were collected from healthy donors by apheresis. Peripheral blood mononuclear cells (PBMCs) were isolated using FICOLL-PAQUE™ PLUS Media, and human T cells were purified from PMBCs using a Pan T cell isolation kit (Miltenyi). The purified T cells were subsequently pre-activated for 48 hours with a human T cell activation/expansion kit (Miltenyi). Following pre-activation, anti-CD3/CD28 MACSiBead™ particles were added at a bead-to-cell ratio of 1:2 to expand the T cells.

The pre-activated T cells were transduced with lentivirus in the presence of 8 μg/mL polybrene. The cells were cultured in 6-well tissue culture plates with 4×10⁶ T cells/well, for approximately 48 hours at 37° C. The transduced cells were centrifuged, the supernatant was removed, and the pelleted cells were resuspended at 0.5×10⁶ cells/mL in fresh media supplemented with 300 IU/ml IL-2 for culture. The cell concentration was adjusted to 0.5×10⁶ cells/mL every 2 to 3 days.

CAR expression was detected on T cells by rabbit-anti-sdAb with the CAR cultured cells, to detect the cell surface sdAbs.

### Example 13: In Vitro Cytotoxicity

This example showed the in vitro anti-tumor activity of anti-BCMA-co-anti-BCMA CAR-T cells.

### 13.1 Lactate Dehydrogenase (LDH) Cytoxicity

For quick evaluation of anti-tumor activities of engineered T cells (e.g., anti-BCMA CAR-γδT and anti-BCMA-co-anti-BCMA CAR-γδT cells) in vitro, a lactate dehydrogenase (LDH) assay for cytotoxicity was performed. On day 7 post-transduction, tandem anti-BCMA-co-anti-BCMA CAR (SEQ ID NO: 61) and single anti-BCMA-co-anti-BCMA CAR (SEQ ID NO: 62) transduced γδT cells were harvested and co-incubated with target cells (BCMA-expressing NCI-H929 or RPMI-8226 tumor cells) at E/T ratio (Effector: CAR-T/Target) ratio of 0.25:1, 0.125:1, 0.0625:1, 0.03125:1 and 0:1, for 20 hours, respectively. Both tandem anti-BCMA CAR (SEQ ID NO: 66) and single anti-BCMA CAR (SEQ ID NO: 67) transduced γδT cells were used as a positive control, untransduced γδT cells (“Un-γδT”) from the same batch were used as a negative control. The assay was performed following the manufacturer's manual (Roche).

The in vitro cytotoxicity was calculated by the equation below ([LDH]_{E+T}: the LDH released from E/T co-incubation, [LDH]_{E}: the LDH released from Effector only, [LDH]ₘₐₓ: the LDH released from target cells treated with Triton X-100, [LDH]ₘᵢₙ: the LDH released from UnTreated target cells):

$$Cytotoxicity \% = \frac{{[ LDH ]}_{E + T} - {[ LDH ]}_{E} - {[ LDH ]}_{min}}{{[ LDH ]}_{max} - {[ LDH ]}_{min}} \times 100 \%$$

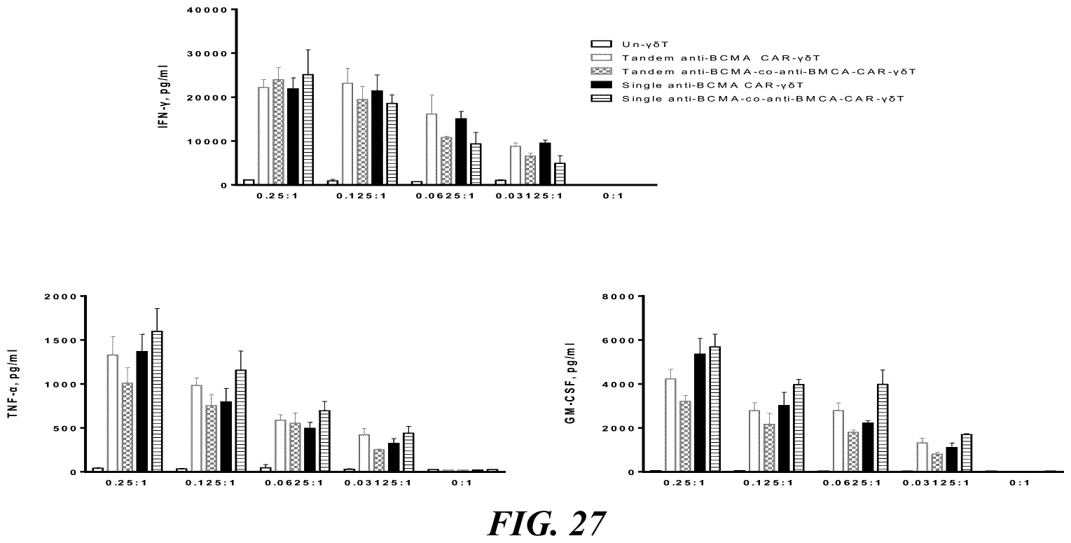

All tested CAR constructs effectively transduced human γδT cells with CAR expression rates between 25% and 80%. Cell growth and viability of transduced cells were not affected relative to untransduced T cells in the same batch of experiments. As shown in FIG. 26, control tandem or single CAR-γδT and tandem or single anti-BCMA-co-anti-BCMA-CAR-γδT cells induced target cell lysis in a dose dependent manner in vitro for both NCI-H929 and RPMI-8226 tumor cells tested. Moreover, anti-BCMA-co-anti-BCMA-CAR-γδT cells exhibited stronger cytotoxicity against target cells than anti-BCMA CAR-γδT for both tandem and single designs.

### 13.2 Cytokine Secretion by Homogeneous Time Resolved Fluorescence (HTRF)

Another measure of effector γδT-cell activation is the production of effector cytokines such as IFN-γ, TNF-α and GM-CSF. Supernatants from the in vitro cytotoxicity assay were collected to assess CAR-induced cytokine release, and Homogeneous Time Resolved Fluorescence (HTRF) assays for IFN-γ, TNF-α and GM-CSF (Cisbio) were performed according to the manufacturer's manual.

CAR-γδT cells (e.g., single/tandem anti-BCMA CAR-γδT and single/tandem anti-BCMA-co-anti-BCMA CAR-γδT cells, and untransduced γδT cells) were co-cultured with NCI-H929 or RPMI-8226 target cells. The culture supernatants were collected after 20 h to assess IFN-γ, TNF-α and GM-CSF release as a measure of γδT cell activation. As shown in FIGS. 27, single anti-BCMA-co-anti-BCMA CAR-γδT cells and tandem anti-BCMA-co-anti-BCMA CAR-γδT cells co-cultured with target tumor cells secreted significant amounts of IFN-γ, TNF-α and GM-CSF, similar to that of anti-BCMA-CAR-γδT cells for both single and tandem sdAb designs. This suggested that the addition of anti-BCMA-co-CAR does not impact CAR-mediated anti-tumor cytotoxicity. Most likely, the TLR4 signaling contribute to enhancing persistence of CAR-γδT cells following target engagement. To test this hypothesis, these cells were subjected to long-term co-culture assay described below.

### 13.3 Long-Term Co-Culture Assay

To evaluate the long-term killing efficacy and persistence of anti-BCMA-co-anti-BCMA CAR-γδT and anti-BCMA-co-anti-GPRC5D CAR-γδT cells, long-term co-culture assays were performed to mimic the dynamic killing process in vivo. Transduced (e.g., tandem anti-BCMA CAR-γδT, tandem anti-BCMA-co-anti-BCMA CAR-γδT cells, single anti-BCMA-co-anti-BCMA CAR-γδT cells, single anti-BCMA-co-anti-BCMA-CD8 CAR-γδT cells, single anti-BCMA-co-anti-BCMA-CD28 CAR-γδT cells and tandem anti-BCMA-co-anti-GPRC5D CAR-γδT cells) or non-transduced (e.g., Un-γδT) T cells (1×10⁵/well) were co-cultured with NCI-H929 target cells (4×10⁵ well) at an E:T ratio of 1:3 in 24-well plates, in the absence of exogenous cytokines (e.g., IL-2). Part of the cells were harvested and stained for CD3 after 2 or 3 days of co-culture. CAR-γδT cells were identified by CD3 and CAR signal. For serial co-culture assays, the remaining γδT cells were then re-challenged with fresh NCI-H929 target cells at the same E:T ratio. Co-cultures were carried on until the tumor cells outgrew the well volume. The γδT cell proliferation rate at each time point was calculated by dividing the number of γδT cells at the time point by the initial number of γδT cells.

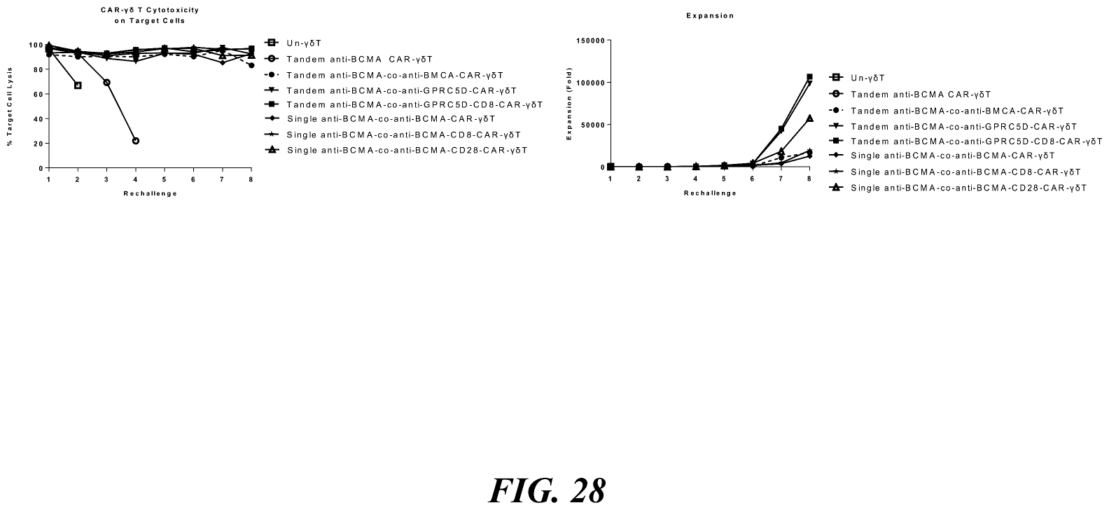

The killing efficacy of various CAR-T cells in the repeated tumor stimulation assay is shown in FIG. 28. Tandem anti-BCMA CAR-γδT cells became exhausted after 4 rounds of tumor stimulation (e.g., could not kill additional target tumor cells, as measured by CD3%), whereas tandem anti-BCMA-co-anti-BCMA CAR-γδT cells, single anti-BCMA-co-anti-BCMA CAR-γδT cells, single anti-BCMA-co-anti-BCMA-CD8 CAR-γδT cells, single anti-BCMA-co-anti-BCMA-CD28 CAR-γδT cells, tandem anti-BCMA-co-anti-GPRC5D CAR-γδT cells and tandem anti-BCMA-co-anti-GPRC5D-CD8 CAR-γδT cells persisted killing target tumor cells to 8 rounds of tumor stimulation. In addition, we found that tandem anti-BCMA-co-anti-GPRC5D CAR-γδT cells proliferated and exhibited better long-term persistence than tandem anti-BCMA CAR-γδT cells in vitro. Moreover, tandem anti-BCMA-co-anti-GPRC5D CAR-γδT cells, tandem anti-BCMA-co-anti-GPRC5D-CD8 CAR-γδT cells and single anti-BCMA-co-anti-BCMA-CD28 CAR-γδT cells showed much better proliferation than tandem anti-BCMA-co-anti-BCMA CAR-γδT cells after 8 rounds of repeated stimulation (FIG. 28).

Taken together, these results demonstrate that TLR4 signaling activation renders CAR-T cells resistant to exhaustion and enhances their ability to kill target tumor cells in vitro, thus addressing major barriers to progress with this class of therapeutic agents. Specifically, we demonstrated that TLR4 signaling triggered by tandem or single anti-BCMA sdAb worked equally well in enhancing anti-tumor cytotoxicity and persistence of CAR-γδT cells. In addition, cell cytotoxicity and persistence and be further improved with anti-GPRC5D scFv or different hinge designs, such as, but not limited to CD28.

### Example 14: In Vitro Persistence in an Allogeneic Setting

This example showed the in vitro persistence of tandem anti-BCMA-co-anti-BCMA CAR-T cells in an allogeneic setting.

### 14.1 Three-Way Mixed Lymphocyte Reaction (MLR) Assay

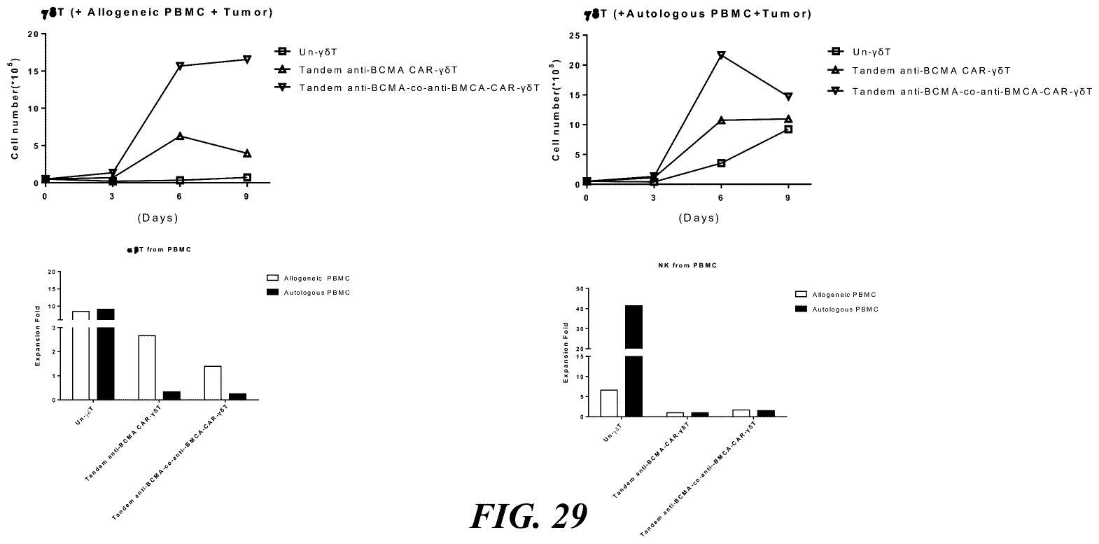

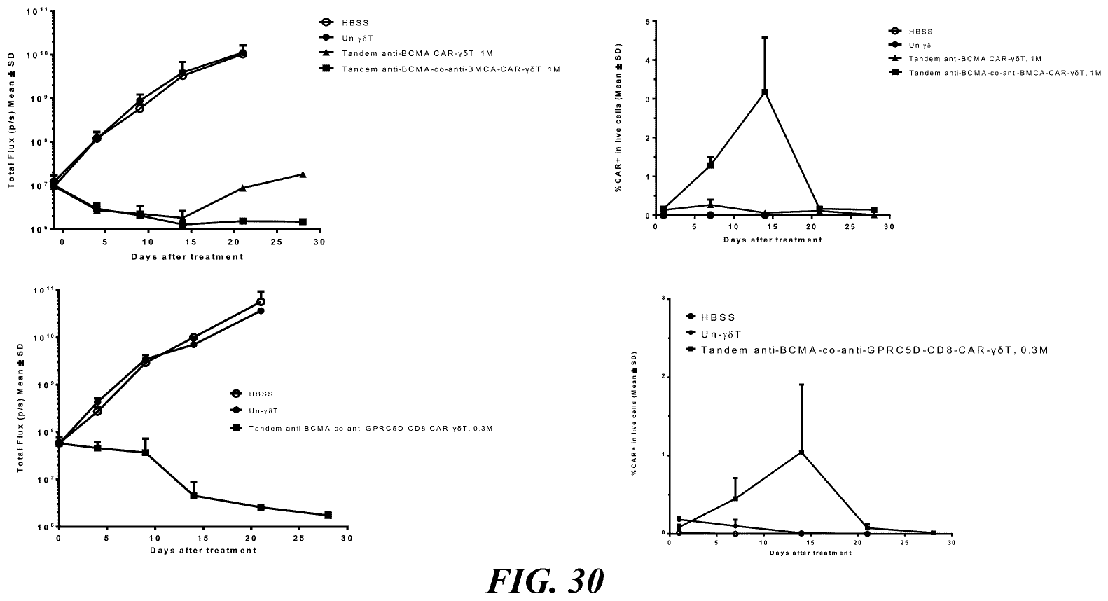

To test the potential of tandem anti-BCMA-co-anti-BCMA CAR-γδT cells as an ‘off-the-shelf’ product for allogeneic cell therapeutic, we mimick the allogeneic environment in vitro by performing a three-way MLR assay with co-culture of CAR-γδT, HCI-H929 tumor cells and allogeneic PBMC (with autologous PBMC as control) in the same system. Specifically, these cells were co-cultured at a ratio of γδT:PBMC:tumor=1:60:1 with an initial γδT cell number of 4×10⁵ cells per well. 3, 6 and 9 days after co-culture, the number of γδT cells were recorded to determine there proliferation in the presence of allogeneic PBMC. On the other hand, on day 9, αβT and NK cell numbers were recorded to determine whether these major effector cell of allogeneic nature were stimulated by γδT cells. As shown in FIG. 29, tandem anti-BCMA-co-anti-BCMA CAR-γδT displayed superior proliferation level compared to tandem anti-BCMA CAR-γδT cells in the presence of allogeneic PBMCs. On the other hand, it was resoundingly clear that the proliferation of allogeneic αβT and NK cells in this system was much less for the tandem anti-BCMA-co-anti-BCMA CAR-γδT cells compared to the control tandem anti-BCMA CAR-γδT cells.

Therefore, we demonstrated that tandem anti-BCMA-co-anti-BCMA CAR-γδT cells equipped with ligand-induced TLR4 signaling was a viable candidate for an “off-the-shelf” allogenic cell therapeutic agent as it displayed superior persistence even in the presence of allogeneic cells.

### Example 15: In Vivo Efficacy and Safety

This example showed the in vivo efficacy and safety of exemplary tandem anti-BCMA CAR-T cells and tandem anti-BCMA-co-anti-BCMA CAR-T cells in an allogeneic setting.

### 15.1 In Vivo Efficacy and Safety in RPMI-8226 Xenograft Model

Anti-tumor activity of an exemplary tandem anti-BCMA CAR-T was assessed in vivo in an RPMI-8226 xenograft model. Briefly, one million (1×10⁶) RPMI-8226 cells stably expressing the firefly luciferase reporter were implanted subcutaneously/intravenously on day 0 in NOD/SCID IL-2R7Cnull (NSG) mice. Fourteen days after tumor inoculation, mice were treated with intravenous injection of 1×10⁶ armored CAR-γδT or mock T cells or phosphate-buffered saline (PBS). Tumor progression was monitored by bioluminescent imaging (BLI) once a week. In addition, T cell proliferation was monitored via FACS analysis from plasma drawn from blood.

For toxicity evaluations, clinical symptoms were observed every day, while the animals' body weights and the fluorescence intensities triggered by tumor-Luc cells were measured every week. Blood (0.2 mL) was taken every week for detecting the humanized cytokine profiles (IFN-γ, TNF-α and GM-CSF) in mice.

In the first experiment with low tumor burden and a dosage of 1M CAR-γδT cells per mice, we found that both tandem anti-BCMA CAR-γδT and tandem anti-BCMA-co-anti-BCMA CAR-γδT cells can inhibit tumor growth. However, it was evident that tandem anti-BCMA-co-anti-BCMA CAR-γδT displayed better anti-tumor activity than tandem anti-BCMA CAR-γδT cells as only the mice treated with tandem anti-BCMA-co-anti-BCMA CAR-γδT cells remained tumor-free while the other experimental group relapsed towards the end of the experiment (FIG. 30). Moreover, pharmacokinetics results from the FACS analysis of mice blood indicated that the expansion of CAR-γδT cells in the peripherical blood was much higher for the tandem anti-BCMA-co-anti-BCMA CAR-γδT group than tandem anti-BCMA CAR-γδT group, further confirming the better in vivo efficacy with tandem anti-BCMA-co-anti-BCMA CAR-γδT cells (FIG. 30).

In another experiment with higher initial tumor burden and a lower dosage of 0.3M CAR-γδT cells per mice, we found tandem anti-BCMA-co-anti-GPRC5D-CD8 CAR-γδT cells was still able to eradicate multiple myeloma tumor cells, thus establishing the feasibility of BCMA and GPRC5D dual targeting and the potential usefulness of different hinge designs, such as, but not limited to CD8 (FIG. 30).

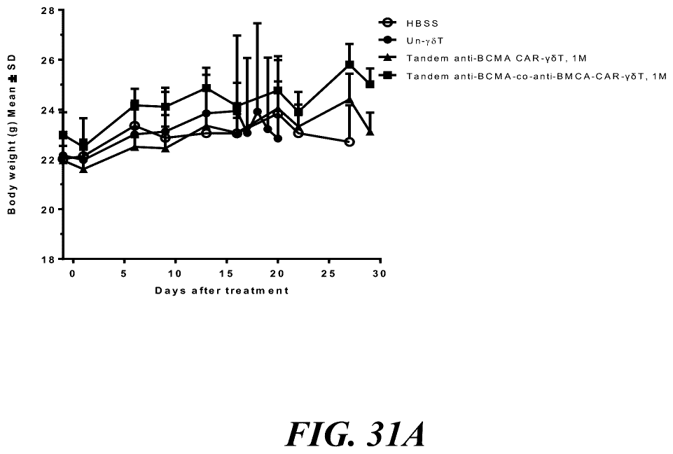

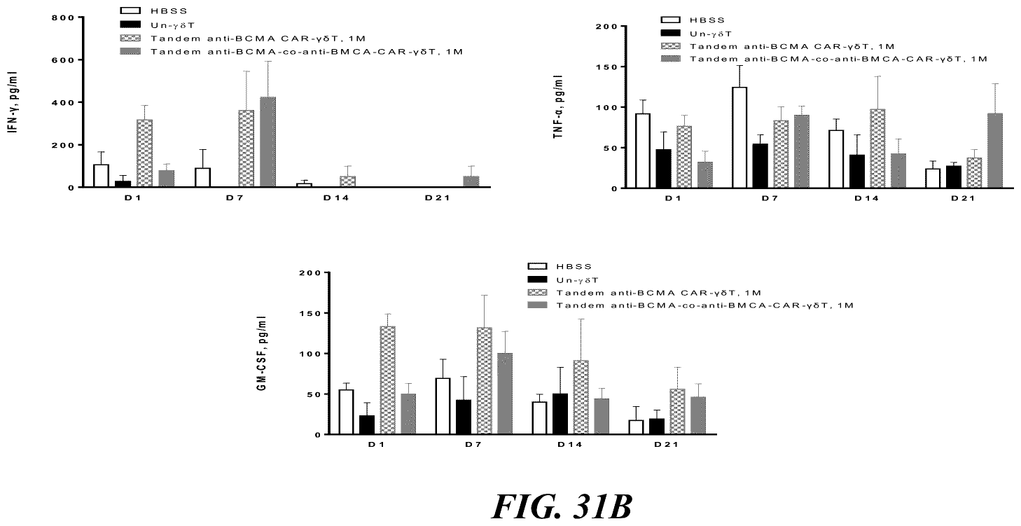

Further, no significant weight change was found between all groups tested. In addition, IFN-γ, TNF-α and GM-CSF levels in the peripheral blood of tandem anti-BCMA-co-anti-BCMA CAR-γδT-treated mice were similar to that of tandem anti-BCMA CAR-γδT-treated mice. These findings were comparable to the observations made in in vitro co-culture assays (FIG. 31A&B). These results suggested that tandem anti-BCMA-co-anti-BCMA CAR-γδT therapy was safe in vivo.

To summarize, we established in in vivo multiple myeloma model that anti-BCMA CAR-γδ T cells equipped with anti-BCMA-triggered-TLR4 signaling were safe and much more efficacious than mere second-generation anti-BCMA CAR-76 T.

SEQUENCE LISTING

SEQ ID NO: 1. CD19-co-CD20-CAR
MALPVTALLLPLALLLHAARPDIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGGTK
LEITGGGGSGGGGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAIYYCAKHYYYG
GSYAMDYWGQGTSVTVSSTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQEEDGCSCRFPEEE
EGGCELRVKFSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
GSGATNFSLLKQAGDVEENPGPMALPVTALLLPLALLLHAARPDIVMTQTPLSSPVTLGQP
ASISCRSSQSLVYSDGNTYLSWLQQRPGQPPRLLIYKISNRFSGVPDRFSGSGAGTDFTLKIS
RVEAEDVGVYYCVQATQFPLTFGGGTKVEIKGGGGSGGGGSGGGGSEVQLVQSGAEVKK
PGESLKISCKGSGYSFTSYWIGWVRQMPGKGLEWMGIIYPGDSDTRYSPSFQGQVTISADKS
ITTAYLQWSSLKASDTAMYYCARHPSYGSGSPNFDYWGQGTLVTVSSTTTPAPRPPTPAPTI
ASQPLSLRPEACRPAAGGAVHTRGLDFACDTIIGVSVLSVLVVSVVAVLVYKFYFHLMLLA
GCIKYGRGENIYDAFVIYSSQDEDWVRNELVKNLEEGVPPFQLCLHYRDFIPGVAIAANIIH
EGFHKSRKVIVVVSQHFIQSRWCIFEYEIAQTWQFLSSRAGIIFIVLQKVEKTLLRQQVELYR
LLSRNTYLEWEDSVLGRHIFWRRLRKALLDGKSWNPEGTVGTGCNWQEATSI
SEQ ID NO: 2. CD19 BM CAR
MALPVTALLLPLALLLHAARPDIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGGTK
LEITGGGGSGGGGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAIYYCAKHYYYG
GSYAMDYWGQGTSVTVSSTSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
R
SEQ ID NO: 3. CLL1-co-CD33 CAR
MALPVTALLLPLALLLHAARPQVQLVESGGGSVQAGGALSLSCAASGYTVRIDYMGWYR
QTPGKGREPVATIASNGGTAYADSVEGRFTISQDNAKNSVYLQMNTLKPGDTAMYYCAA
GTWPTLTYFGQGTQVTVSSIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRDFA
AYRSRVKFSRSADAPAYKQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPRGS
GATNFSLLKQAGDVEENPGPMALPVTALLLPLALLLHAARPEVQLVESGGGSVQAGGSLR
LSCAASGYTYSINCMGWFRQAPGKEREGVAVISTGGGRTDYRDSVKGRFTISQDNAKNTV
YLQMNSLKPEDTAMYYCAGKTTYPGYGCGLGRSAYNYWGQGTQVTVSSLSVSECHRTAL
VSGMCCALFLLILLTGVLCHRFHGLWYMKMMWAWLQAKRKPRKAPSRNICYDAFVSYSE
RDAYWVENLMVQELENFNPPFKLCLHKRDFIPGKWIIDNIIDSIEKSHKTVFVLSENFVKSE
WCKYELDFSHFRLFDENNDAAILILLEPIEKKAIPQRFCKLRKIMNTKTYLEWPMDEAQREG
FWVNLRAAIKSGSGATNFSLLKQAGDVEENPGPMALPVTALLLPLALLLHAARPQVQLVES
GGGLVQAGGSLRLSCAASGNVFRFNIMGWYRQAPGNQRELVASIDDGGDRSYADSVEGRF
TISRENGKKIMYLQMNSLKPEDTAVYYCAAGLGTYLNGRVSMATNYWGQGTQVTVSSLS
CNITLLIVTIVATMLVLAVTVTSLCSYLDLPWYLRMVCQWTQTRRRARNIPLEELQRNLQF
HAFISYSGHDSFWVKNELLPNLEKEGMQICLHERNFVPGKSIVENIITCIEKSYKSIFVLSPNF
VQSEWCHYELYFAHHNLFHEGSNSLILILLEPIPQYSIPSSYHKLKSLMARRTYLEWPKEKS
KRGLFWANLRAAINIKLTEQAKK
SEQ ID NO: 4. CLL1 BM CAR
MALPVTALLLPLALLLHAARPQVQLVESGGGSVQAGGALSLSCAASGYTVRIDYMGWYR
QTPGKGREPVATIASNGGTAYADSVEGRFTISQDNAKNSVYLQMNTLKPGDTAMYYCAA
GTWPTLTYFGQGTQVTVSSIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRDFA
AYRSRVKFSRSADAPAYKQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 5. Signal Peptide (e.g., Leader Sequence)
MALPVTALLLPLALLLHAARP
SEQ ID NO: 6. CD19 scFv
DIQMTQTTSSLSASLGDRVTISCRASQDISKYLNWYQQKPDGTVKLLIYHTSRLHSGVPSRF
SGSGSGTDYSLTISNLEQEDIATYFCQQGNTLPYTFGGGTKLEITGGGGSGGGGSGGGGSEV
KLQESGPGLVAPSQSLSVTCTVSGVSLPDYGVSWIRQPPRKGLEWLGVIWGSETTYYNSAL
KSRLTIIKDNSKSQVFLKMNSLQTDDTAIYYCAKHYYYGGSYAMDYWGQGTSVTVSS
SEQ ID NO: 7. CD8α hinge
TTTPAPRPPTPAPTIASQPLSLRPEACRPAAGGAVHTRGLDFACD
SEQ ID NO: 8. CD8α TM
IYIWAPLAGTCGVLLLSLVITLYC
SEQ ID NO: 9. 4-1BB (CD137) co-stimulatory signaling domain
KRGRKKLLYIFKQPFMRPVQTTQEEDGCSCRFPEEEEGGCEL
SEQ ID NO: 10. CD3ζ primary intracellular signaling domain
RVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
SEQ ID NO: 11. CD20 scFv
DIVMTQTPLSSPVTLGQPASISCRSSQSLVYSDGNTYLSWLQQRPGQPPRLLIYKISNRFSGVPDRFSG
SGAGTDFTLKISRVEAEDVGVYYCVQATQFPLTFGGGTKVEIKGGGGSGGGGSGGGGSEVQLVQSG
AEVKKPGESLKISCKGSGYSFTSYWIGWVRQMPGKGLEWMGIIYPGDSDTRYSPSFQGQVTISADKS
ITTAYLQWSSLKASDTAMYYCARHPSYGSGSPNFDYWGQGTLVTVSS
SEQ ID NO: 12. TLR4 TM
TIIGVSVLSVLVVSVVAVLVY
SEQ ID NO: 13. TLR4 primary intracellular signaling domain
KFYFHLMLLAGCIKYGRGENIYDAFVIYSSQDEDWVRNELVKNLEEGVPPFQLCLHYRDF
SEQ ID NO: 14. CLL1 sdAb
QVQLVESGGGSVQAGGALSLSCAASGYTVRIDYMGWYRQTPGKGREPVATIASNGGTAY
ADSVEGRFTISQDNAKNSVYLQMNTLKPGDTAMYYCAAGTWPTLTYFGQGTQVTVSS
SEQ ID NO: 15. CD28α hinge
IEVMYPPPYLDNEKSNGTIIHVKGKHLCPSPLFPGPSKP
SEQ ID NO: 16. CD28α TM
FWVLVVVGGVLACYSLLVTVAFIIFWV
SEQ ID NO: 17. CD28 primary intracellular signaling domain
RSKRSRLLHSDYMNMTPRRPGPTRKHYQPYAPPRDFAAYRS
SEQ ID NO: 18. CD33 C2 domain sdAb
QVQLVESGGGLVQAGGSLRLSCAASGNVFRENIMGWYRQAPGNQRELVASIDDGGDRSY
ADSVEGRFTISRENGKKIMYLQMNSLKPEDTAVYYCAAGLGTYLNGRVSMATNYWGQGT
QVTVSS
SEQ ID NO: 19. TLR1 TM
LLIVTIVATMLVLAVTVTSLC
SEQ ID NO: 20. TLR1 primary intracellular signaling domain
SYLDLPWYLRMVCQWTQTRRRARNIPLEELQRNLQFHAFISYSGHDSFWVKNELLPNLEK
EGMQICLHERNFVPGKSIVENIITCIEKSYKSIFVLSPNFVQSEWCHYELYFAHHNLFHE
GSNSLILILLEPIPQYSIPSSYHKLKSLMARRTYLEWPKEKSKRGLFWANLRAAINIKLT
EQAKK
SEQ ID NO: 21. CD33 V domain sdAb
EVQLVESGGGSVQAGGSLRLSCAASGYTYSINCMGWFRQAPGKEREGVAVISTGGGRTDY
RDSVKGRFTISQDNAKNTVYLQMNSLKPEDTAMYYCAGKTTYPGYGCGLGRSAYNYWG
QGTQVTVSS
SEQ ID NO: 22. TLR2 TM
ALVSGMCCALFLLILLTGVLC
SEQ ID NO: 23. TLR2 primary intracellular signaling domain
HRFHGLWYMKMMWAWLQAKRKPRKAPSRNICYDAFVSYSERDAYWVENLMVQELENF
NPP
SEQ ID NO: 24. P2A
GSGATNFSLLKQAGDVEENPGP
SEQ ID NO: 25. GS linker 1
GGGGSGGGGSGGGGSGGGGS
SEQ ID NO: 26. GS linker 2
GGGGS
SEQ ID NO: 27. GS linker 3
(GS)ₙ
SEQ ID NO: 28. GS linker 4
(GSGGS)ₙ
SEQ ID NO: 29. GS linker 5
(GGGS)ₙ
SEQ ID NO: 30. TLR1
MTSIFHFAIIFMLILQIRIQLSEESEFLVDRSKNGLIHVPKDLSQKTTILNISQNYISELWTSDIL
SLSKLRILIISHNRIQYLDISVFKFNQELEYLDLSHNKLVKISCHPTVNLKHLDLSFNAFDALPI
CKEFGNMSQLKFLGLSTTHLEKSSVLPIAHLNISKVLLVLGETYGEKEDPEGLQDENTESLHI
VFPTNKEFHFILDVSVKTVANLELSNIKCVLEDNKCSYFLSILAKLQTNPKLSNLTLNNIETT
WNSFIRILQLVWHTTVWYFSISNVKLQGQLDFRDFDYSGTSLKALSIHQVVSDVFGFPQSYI
YEIFSNMNIKNFTVSGTRMVHMLCPSKISPFLHLDFSNNLLTDTVFENCGHLTELETLILQM
NQLKELSKIAEMTTQMKSLQQLDISQNSVSYDEKKGDCSWTKSLLSLNMSSNILTDTIFRCL
PPRIKVLDLHSNKIKSIPKQVVKLEALQELNVAFNSLTDLPGCGSFSSLSVLIIDHNSVSHPSA
DFFQSCQKMRSIKAGDNPFQCTCELGEFVKNIDQVSSEVLEGWPDSYKCDYPESYRGTLLK
DFHMSELSCNITLLIVTIVATMLVLAVTVTSLCSYLDLPWYLRMVCQWTQTRRRARNIPLE
ELQRNLQFHAFISYSGHDSFWVKNELLPNLEKEGMQICLHERNFVPGKSIVENIITCIEKSYK
SIFVLSPNFVQSEWCHYELYFAHHNLFHEGSNSLILILLEPIPQYSIPSSYHKLKSLMARRTYL
EWPKEKSKRGLFWANLRAAINIKLTEQAKK
SEQ ID NO: 31. TLR2
MPHTLWMVWVLGVIISLSKEESSNQASLSCDRNGICKGSSGSLNSIPSGLTEAVKSLDLSNN
RITYISNSDLQRCVNLQALVLTSNGINTIEEDSFSSLGSLEHLDLSYNYLSNLSSSWFKPLSSL
TFLNLLGNPYKTLGETSLFSHLTKLQILRVGNMDTFTKIQRKDFAGLTFLEELEIDASDLQSY
EPKSLKSIQNVSHLILHMKQHILLLEIFVDVTSSVECLELRDTDLDTFHFSELSTGETNSLIKK
FTFRNVKITDESLFQVMKLLNQISGLLELEFDDCTLNGVGNFRASDNDRVIDPGKVETLTIR
RLHIPRFYLFYDLSTLYSLTERVKRITVENSKVFLVPCLLSQHLKSLEYLDLSENLMVEEYLK
NSACEDAWPSLQTLILRQNHLASLEKTGETLLTLKNLTNIDISKNSFHSMPETCQWPEKMK
YLNLSSTRIHSVTGCIPKTLEILDVSNNNLNLFSLNLPQLKELYISRNKLMTLPDASLLPMLL
VLKISRNAITTFSKEQLDSFHTLKTLEAGGNNFICSCEFLSFTQEQQALAKVLIDWPANYLCD
SPSHVRGQQVQDVRLSVSECHRTALVSGMCCALFLLILLTGVLCHRFHGLWYMKMMWA
WLQAKRKPRKAPSRNICYDAFVSYSERDAYWVENLMVQELENFNPPFKLCLHKRDFIPGK
WIIDNIIDSIEKSHKTVFVLSENFVKSEWCKYELDFSHFRLFDENNDAAILILLEPIEKKAIPQR
FCKLRKIMNTKTYLEWPMDEAQREGFWVNLRAAIKS
SEQ ID NO: 32. TLR3
MRQTLPCIYFWGGLLPFGMLCASSTTKCTVSHEVADCSHLKLTQVPDDLPTNITVLNLTHN
QLRRLPAANFTRYSQLTSLDVGFNTISKLEPELCQKLPMLKVLNLQHNELSQLSDKTFAFCT
NLTELHLMSNSIQKIKNNPFVKQKNLITLDLSHNGLSSTKLGTQVQLENLQELLLSNNKIQA
LKSEELDIFANSSLKKLELSSNQIKEFSPGCFHAIGRLFGLFLNNVQLGPSLTEKLCLELANTS
IRNLSLSNSQLSTTSNTTFLGLKWTNLTMLDLSYNNLNVVGNDSFAWLPQLEYFFLEYNNI
QHLFSHSLHGLFNVRYLNLKRSFTKQSISLASLPKIDDFSFQWLKCLEHLNMEDNDIPGIKSN
MFTGLINLKYLSLSNSFTSLRTLTNETFVSLAHSPLHILNLTKNKISKIESDAFSWLGHLEVLD
LGLNEIGQELTGQEWRGLENIFEIYLSYNKYLQLTRNSFALVPSLQRLMLRRVALKNVDSSP
SPFQPLRNLTILDLSNNNIANINDDMLEGLEKLEILDLQHNNLARLWKHANPGGPIYFLKGL
SHLHILNLESNGFDEIPVEVFKDLFELKIIDLGLNNLNTLPASVFNNQVSLKSLNLQKNLITSV
EKKVFGPAFRNLTELDMRFNPFDCTCESIAWFVNWINETHTNIPELSSHYLCNTPPHYHGFP
VRLFDTSSCKDSAPFELFFMINTSILLIFIFIVLLIHFEGWRISFYWNVSVHRVLGFKEIDRQTE
QFEYAAYIIHAYKDKDWVWEHFSSMEKEDQSLKFCLEERDFEAGVFELEAIVNSIKRSRKII
FVITHHLLKDPLCKRFKVHHAVQQAIEQNLDSIILVFLEEIPDYKLNHALCLRRGMFKSHCIL
NWPVQKERIGAFRHKLQVALGSKNSVH
SEQ ID NO: 33. TLR4
MMSASRLAGTLIPAMAFLSCVRPESWEPCVEVVPNITYQCMELNFYKIPDNLPFSTKNLDLS
FNPLRHLGSYSFFSFPELQVLDLSRCEIQTIEDGAYQSLSHLSTLILTGNPIQSLALGAFSGLSS
LQKLVAVETNLASLENFPIGHLKTLKELNVAHNLIQSFKLPEYFSNLTNLEHLDLSSNKIQSI
YCTDLRVLHQMPLLNLSLDLSLNPMNFIQPGAFKEIRLHKLTLRNNFDSLNVMKTCIQGLA
GLEVHRLVLGEFRNEGNLEKFDKSALEGLCNLTIEEFRLAYLDYYLDDIIDLFNCLTNVSSFS
LVSVTIERVKDFSYNFGWQHLELVNCKFGQFPTLKLKSLKRLTFTSNKGGNAFSEVDLPSLE
FLDLSRNGLSFKGCCSQSDFGTTSLKYLDLSFNGVITMSSNFLGLEQLEHLDFQHSNLKQMS
EFSVFLSLRNLIYLDISHTHTRVAFNGIFNGLSSLEVLKMAGNSFQENFLPDIFTELRNLTFLD
LSQCQLEQLSPTAFNSLSSLQVLNMSHNNFFSLDTFPYKCLNSLQVLDYSLNHIMTSKKQEL
QHFPSSLAFLNLTQNDFACTCEHQSFLQWIKDQRQLLVEVERMECATPSDKQGMPVLSLNI
TCQMNKTIIGVSVLSVLVVSVVAVLVYKFYFHLMLLAGCIKYGRGENIYDAFVIYSSQDED
WVRNELVKNLEEGVPPFQLCLHYRDFIPGVAIAANIIHEGFHKSRKVIVVVSQHFIQSRWCIF
EYEIAQTWQFLSSRAGIIFIVLQKVEKTLLRQQVELYRLLSRNTYLEWEDSVLGRHIFWRRL
RKALLDGKSWNPEGTVGTGCNWQEATSI
SEQ ID NO: 34. TLR5
MGDHLDLLLGVVLMAGPVFGIPSCSFDGRIAFYRFCNLTQVPQVLNTTERLLLSFNYIRTVT
ASSFPFLEQLQLLELGSQYTPLTIDKEAFRNLPNLRILDLGSSKIYFLHPDAFQGLFHLFELRL
YFCGLSDAVLKDGYFRNLKALTRLDLSKNQIRSLYLHPSFGKLNSLKSIDFSSNQIFLVCEHE
LEPLQGKTLSFFSLAANSLYSRVSVDWGKCMNPFRNMVLEILDVSGNGWTVDITGNFSNAI
SKSQAFSLILAHHIMGAGFGFHNIKDPDQNTFAGLARSSVRHLDLSHGFVFSLNSRVFETLK
DLKVLNLAYNKINKIADEAFYGLDNLQVLNLSYNLLGELYSSNFYGLPKVAYIDLQKNHIA
IIQDQTFKFLEKLQTLDLRDNALTTIHFIPSIPDIFLSGNKLVTLPKINLTANLIHLSENRLENL
DILYFLLRVPHLQILILNQNRFSSCSGDQTPSENPSLEQLFLGENMLQLAWETELCWDVFEG
LSHLQVLYLNHNYLNSLPPGVFSHLTALRGLSLNSNRLTVLSHNDLPANLEILDISRNQLLA
PNPDVFVSLSVLDITHNKFICECELSTFINWLNHTNVTIAGPPADIYCVYPDSFSGVSLFSLST
EGCDEEEVLKSLKFSLFIVCTVTLTLFLMTILTVTKFRGFCFICYKTAQRLVFKDHPQGTEPD
MYKYDAYLCFSSKDFTWVQNALLKHLDTQYSDQNRFNLCFEERDFVPGENRIANIQDAIW
NSRKIVCLVSRHFLRDGWCLEAFSYAQGRCLSDLNSALIMVVVGSLSQYQLMKHQSIRGFV
QKQQYLRWPEDFQDVGWFLHKLSQQILKKEKEKKKDNNIPLQTVATIS
SEQ ID NO: 35. TLR6
MTKDKEPIVKSFHFVCLMIIIVGTRIQFSDGNEFAVDKSKRGLIHVPKDLPLKTKVLDMSQN
YIAELQVSDMSFLSELTVLRLSHNRIQLLDLSVFKFNQDLEYLDLSHNQLQKISCHPIVSFRH
LDLSFNDFKALPICKEFGNLSQLNFLGLSAMKLQKLDLLPIAHLHLSYILLDLRNYYIKENET
ESLQILNAKTLHLVFHPTSLFAIQVNISVNTLGCLQLTNIKLNDDNCQVFIKFLSELTRGSTLL
NFTLNHIETTWKCLVRVFQFLWPKPVEYLNIYNLTIIESIREEDFTYSKTTLKALTIEHITNQV
FLFSQTALYTVFSEMNIMMLTISDTPFIHMLCPHAPSTFKFLNFTQNVFTDSIFEKCSTLVKLE
TLILQKNGLKDLFKVGLMTKDMPSLEILDVSWNSLESGRHKENCTWVESIVVLNLSSNMLT
DSVFRCLPPRIKVLDLHSNKIKSVPKQVVKLEALQELNVAFNSLTDLPGCGSFSSLSVLIIDH
NSVSHPSADFFQSCQKMRSIKAGDNPFQCTCELREFVKNIDQVSSEVLEGWPDSYKCDYPE
SYRGSPLKDFHMSELSCNITLLIVTIGATMLVLAVTVTSLCIYLDLPWYLRMVCQWTQTRR
RARNIPLEELQRNLQFHAFISYSEHDSAWVKSELVPYLEKEDIQICLHERNFVPGKSIVENIIN
CIEKSYKSIFVLSPNFVQSEWCHYELYFAHHNLFHEGSNNLILILLEPIPQNSIPNKYHKLKAL
MTQRTYLQWPKEKSKRGLFWANIRAAFNMKLTLVTENNDVKS
SEQ ID NO: 36. TLR7
MVFPMWTLKRQILILFNIILISKLLGARWFPKTLPCDVTLDVPKNHVIVDCTDKHLTEIPGGI
PTNTTNLTLTINHIPDISPASFHRLDHLVEIDFRCNCVPIPLGSKNNMCIKRLQIKPRSFSGLTY
LKSLYLDGNQLLEIPQGLPPSLQLLSLEANNIFSIRKENLTELANIEILYLGQNCYYRNPCYVS
YSIEKDAFLNLTKLKVLSLKDNNVTAVPTVLPSTLTELYLYNNMIAKIQEDDFNNLNQLQIL
DLSGNCPRCYNAPFPCAPCKNNSPLQIPVNAFDALTELKVLRLHSNSLQHVPPRWFKNINKL
QELDLSQNFLAKEIGDAKFLHELPSLIQLDLSFNFELQVYRASMNLSQAFSSLKSLKILRIRG
YVFKELKSFNLSPLHNLQNLEVLDLGTNFIKIANLSMFKQFKRLKVIDLSVNKISPSGDSSEV
GFCSNARTSVESYEPQVLEQLHYFRYDKYARSCRFKNKEASFMSVNESCYKYGQTLDLSK
NSIFFVKSSDFQHLSFLKCLNLSGNLISQTLNGSEFQPLAELRYLDFSNNRLDLLHSTAFEEL
HKLEVLDISSNSHYFQSEGITHMLNFTKNLKVLQKLMMNDNDISSSTSRTMESESLRTLEFR
GNHLDVLWREGDNRYLQLFKNLLKLEELDISKNSLSFLPSGVFDGMPPNLKNLSLAKNGLK
SFSWKKLQCLKNLETLDLSHNQLTTVPERLSNCSRSLKNLILKNNQIRSLTKYFLQDAFQLR
YLDLSSNKIQMIQKTSFPENVLNNLKMLLLHHNRFLCTCDAVWFVWWVNHTEVTIPYLAT
DVTCVGPGAHKGQSVISLDLYTCELDLTNLILFSLSISVSLFLMVMMTASHLYFWDVWYIY
HFCKAKIKGYQRLISPDCCYDAFIVYDTKDPAVTEWVLAELVAKLEDPREKHENLCLEERD
WLPGQPVLENLSQSIQLSKKTVFVMTDKYAKTENFKIAFYLSHQRLMDEKVDVIILIFLEKP
FQKSKFLQLRKRLCGSSVLEWPTNPQAHPYFWQCLKNALATDNHVAYSQVFKETV
SEQ ID NO: 37. TLR8
VGKYVTELDLSDNFITHITNESFQGLQNLTKINLNHNPNVQHQNGNPGIQSNGLNITDGAFL
NLKNLRELLLEDNQLPQIPSGLPESLTELSLIQNNIYNITKEGISRLINLKNLYLAWNCYFNK
VCEKTNIEDGVFETLTNLELLSLSFNSLSHVPPKLPSSLRKLFLSNTQIKYISEEDFKGLINLTL
LDLSGNCPRCFNAPFPCVPCDGGASINIDRFAFQNLTQLRYLNLSSTSLRKINAAWFKNMPH
LKVLDLEFNYLVGEIASGAFLTMLPRLEILDLSFNYIKGSYPQHINISRNFSKLLSLRALHLRG
YVFQELREDDFQPLMQLPNLSTINLGINFIKQIDFKLFQNFSNLEIIYLSENRISPLVKDTRQSY
ANSSSFQRHIRKRRSTDFEFDPHSNFYHFTRPLIKPQCAAYGKALDLSLNSIFFIGPNQFENLP
DIACLNLSANSNAQVLSGTEFSAIPHVKYLDLTNNRLDFDNASALTELSDLEVLDLSYNSHY
FRIAGVTHHLEFIQNFTNLKVLNLSHNNIYTLTDKYNLESKSLVELVFSGNRLDILWNDDDN
RYISIFKGLKNLTRLDLSLNRLKHIPNEAFLNLPASLTELHINDNMLKFFNWTLLQQFPRLEL
LDLRGNKLLFLTDSLSDFTSSLRTLLLSHNRISHLPSGFLSEVSSLKHLDLSSNLLKTINKSAL
ETKTTTKLSMLELHGNPFECTCDIGDFRRWMDEHLNVKIPRLVDVICASPGDQRGKSIVSLE
LTTCVSDVTAVILFFFTFFITTMVMLAALAHHLFYWDVWFIYNVCLAKVKGYRSLSTSQTF
YDAYISYDTKDASVTDWVINELRYHLEESRDKNVLLCLEERDWDPGLAIIDNLMQSINQSK
KTVFVLTKKYAKSWNFKTAFYLALQRLMDENMDVIIFILLEPVLQHSQYLRLRQRICKSSIL
QWPDNPKAEGLFWQTLRNVVLTENDSRYNNMYVDSIKQY
SEQ ID NO: 38. TLR9
MGFCRSALHPLSLLVQAIMLAMTLALGTLPAFLPCELQPHGLVNCNWLFLKSVPHFSMAAP
RGNVTSLSLSSNRIHHLHDSDFAHLPSLRHLNLKWNCPPVGLSPMHFPCHMTIEPSTFLAVP
TLEELNLSYNNIMTVPALPKSLISLSLSHTNILMLDSASLAGLHALRFLFMDGNCYYKNPCR
QALEVAPGALLGLGNLTHLSLKYNNLTVVPRNLPSSLEYLLLSYNRIVKLAPEDLANLTAL
RVLDVGGNCRRCDHAPNPCMECPRHFPQLHPDTFSHLSRLEGLVLKDSSLSWLNASWFRG
LGNLRVLDLSENFLYKCITKTKAFQGLTQLRKLNLSFNYQKRVSFAHLSLAPSFGSLVALKE
LDMHGIFFRSLDETTLRPLARLPMLQTLRLQMNFINQAQLGIFRAFPGLRYVDLSDNRISGA
SELTATMGEADGGEKVWLQPGDLAPAPVDTPSSEDFRPNCSTLNFTLDLSRNNLVTVQPE
MFAQLSHLQCLRLSHNCISQAVNGSQFLPLTGLQVLDLSHNKLDLYHEHSFTELPRLEALD
LSYNSQPFGMQGVGHNFSFVAHLRTLRHLSLAHNNIHSQVSQQLCSTSLRALDFSGNALGH
MWAEGDLYLHFFQGLSGLIWLDLSQNRLHTLLPQTLRNLPKSLQVLRLRDNYLAFFKWWS
LHFLPKLEVLDLAGNQLKALTNGSLPAGTRLRRLDVSCNSISFVAPGFFSKAKELRELNLSA
NALKTVDHSWFGPLASALQILDVSANPLHCACGAAFMDFLLEVQAAVPGLPSRVKCGSPG
QLQGLSIFAQDLRLCLDEALSWDCFALSLLAVALGLGVPMLHHLCGWDLWYCFHLCLAW
LPWRGRQSGRDEDALPYDAFVVFDKTQSAVADWVYNELRGQLEECRGRWALRLCLEERD
WLPGKTLFENLWASVYGSRKTLFVLAHTDRVSGLLRASFLLAQQRLLEDRKDVVVLVILSP
DGRRSRYVRLRQRLCRQSVLLWPHQPSGQRSFWAQLGMALTRDNHHFYNRNFCQGPTAE
SEQ ID NO: 39. TLR10
MRLIRNIYIFCSIVMTAEGDAPELPEERELMTNCSNMSLRKVPADLTPATTTLDLSYNLLFQ
LQSSDFHSVSKLRVLILCHNRIQQLDLKTFEFNKELRYLDLSNNRLKSVTWYLLAGLRYLD
LSFNDFDTMPICEEAGNMSHLEILGLSGAKIQKSDFQKIAHLHLNTVFLGFRTLPHYEEGSLP
ILNTTKLHIVLPMDTNFWVLLRDGIKTSKILEMTNIDGKSQFVSYEMQRNLSLENAKTSVLL
LNKVDLLWDDLFLILQFVWHTSVEHFQIRNVTFGGKAYLDHNSFDYSNTVMRTIKLEHVH
FRVFYIQQDKIYLLLTKMDIENLTISNAQMPHMLFPNYPTKFQYLNFANNILTDELFKRTIQL
PHLKTLILNGNKLETLSLVSCFANNTPLEHLDLSQNLLQHKNDENCSWPETVVNMNLSYNK
LSDSVFRCLPKSIQILDLNNNQIQTVPKETIHLMALRELNIAFNFLTDLPGCSHFSRLSVLNIE
MNFILSPSLDFVQSCQEVKTLNAGRNPFRCTCELKNFIQLETYSEVMMVGWSDSYTCEYPL
NLRGTRLKDVHLHELSCNTALLIVTIVVIMLVLGLAVAFCCLHFDLPWYLRMLGQCTQTW
HRVRKTTQEQLKRNVRFHAFISYSEHDSLWVKNELIPNLEKEDGSILICLYESYFDPGKSISE
NIVSFIEKSYKSIFVLSPNFVQNEWCHYEFYFAHHNLFHENSDHIILILLEPIPFYCIPTRYHKL
KALLEKKAYLEWPKDRRKCGLFWANLRAAINVNVLATREMYELQTFTELNEESRGSTISL
MRTDCL
SEQ ID NO: 40. Exemplary TLR polypeptide 1 (TLR4)
TIIGVSVLSVLVVSVVAVLVYKFYFHLMLLAGCIKYGRGENIYDAFVIYSSQDEDWVRNEL
VKNLEEGVPPFQLCLHYRDFIPGVAIAANIIHEGFHKSRKVIVVVSQHFIQSRWCIFEYEIAQT
WQFLSSRAGIIFIVLQKVEKTLLRQQVELYRLLSRNTYLEWEDSVLGRHIFWRRLRKALLD
GKSWNPEGTVGTGCNWQEATSI
SEQ ID NO: 41. Exemplary TLR polypeptide 2 (TLR2)
LSVSECHRTALVSGMCCALFLLILLTGVLCHRFHGLWYMKMMWAWLQAKRKPRKAPSR
NICYDAFVSYSERDAYWVENLMVQELENFNPPFKLCLHKRDFIPGKWIIDNIIDSIEKSHKTV
FVLSENFVKSEWCKYELDFSHFRLFDENNDAAILILLEPIEKKAIPQRFCKLRKIMNTKTYLE
WPMDEAQREGFWVNLRAAIKS
SEQ ID NO: 42. Exemplary TLR polypeptide 3 (TLR1)
LSCNITLLIVTIVATMLVLAVTVTSLCSYLDLPWYLRMVCQWTQTRRRARNIPLEELQRNL
QFHAFISYSGHDSFWVKNELLPNLEKEGMQICLHERNFVPGKSIVENIITCIEKSYKSIFVLSP
NFVQSEWCHYELYFAHHNLFHEGSNSLILILLEPIPQYSIPSSYHKLKSLMARRTYLEWPKE
KSKRGLFWANLRAAINIKLTEQAKK
SEQ ID NO: 43. CLL1/CD33 dual CAR
MALPVTALLLPLALLLHAARPQVQLVESGGGSVQAGGALSLSCAASGYTVRIDYMGWYR
QTPGKGREPVATIASNGGTAYADSVEGRFTISQDNAKNSVYLQMNTLKPGDTAMYYCAA
GTWPTLTYFGQGTQVTVSSGGGGSEVQLVESGGGSVQAGGSLRLSCAASGYTYSINCMGW
FRQAPGKEREGVAVISTGGGRTDYRDSVKGRFTISQDNAKNTVYLQMNSLKPEDTAMYYC
AGKTTYPGYGCGLGRSAYNYWGQGTQVTVSSTTTPAPRPPTPAPTIASQPLSLRPEACRPA
AGGAVHTRGLDFACDIYIWAPLAGTCGVLLLSLVITLYCKRGRKKLLYIFKQPFMRPVQTT
QEEDGCSCRFPEEEEGGCELRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKD
TYDALHMQALPPR
SEQ ID NO: 44. Tandem anti-BCMA sdAb
AVQLVESGGGLVQAGDSLRLTCTASGRAFSTYFMAWFRQAPGKEREFVAGIAWSGGSTAY
ADSVKGRFTISRDNAKNTVYLQMNSLKSEDTAVYYCASRGIEVEEFGAWGQGTQVTVSSG
GGGSQVQLEESGGGSVQAGGSLRLSCAYTYSTYSNYYMGWFREAPGKARTSVAIISSDTTI
TYKDAVKGRFTISKDNAKNTLYLQMNSLKPEDSAMYRCAAWTSDWSVAYWGQGTQVTV
SS
SEQ ID NO: 45. GM-CSF signal peptide
MWLQSLLLLGTVACSIS
SEQ ID NO: 46. IL-15 signal peptide
MRISKPHLRSISIQCYLCLLLNSHFLTEA
SEQ ID NO: 47. FLAG tag
DYKDDDDK
SEQ ID NO: 48. HA tag
YPYDVPDYA
SEQ ID NO: 49. c-Myc tag
EQKLISEEDL
SEQ ID NO: 50. CD28 4-1BB intracellular signaling domain
RSKRSRLLHSDYMNMTPRRPGPTRKHYQPYAPPRDFAAYRSKRGRKKLLYIFKQPFMRPV
QTTQEEDGCSCRFPEEEEGGCEL
SEQ ID NO: 51. NKG2D 81-216
SLFNQEVQIPLTESYCGPCPKNWICYKNNCYQFFDESKNWYESQASCMSQNASLLKVYSKE
DQDLLKLVKSYHWMGLVHIPTNGSWQWEDGSILSPNLLTIIEMQKGDCALYASSFKGYIEN
CSTPNTYICMQRTV
SEQ ID NO: 52. NKG2D 89-216
IPLTESYCGPCPKNWICYKNNCYQFFDESKNWYESQASCMSQNASLLKVYSKEDQDLLKL
VKSYHWMGLVHIPTNGSWQWEDGSILSPNLLTIIEMQKGDCALYASSFKGYIENCSTPNTYI
CMQRTV
SEQ ID NO: 53. NKG2D 98-216
PCPKNWICYKNNCYQFFDESKNWYESQASCMSQNASLLKVYSKEDQDLLKLVKSYHWM
GLVHIPTNGSWQWEDGSILSPNLLTIIEMQKGDCALYASSFKGYIENCSTPNTYICMQRTV
SEQ ID NO: 54. NKG2D 1-216
MGWIRGRRSRHSWEMSEFHNYNLDLKKSDFSTRWQKQRCPVVKSKCRENASPFFFCCFIA
VAMGIRFIIMVAIWSAVFLNSLFNQEVQIPLTESYCGPCPKNWICYKNNCYQFFDESKNWY
ESQASCMSQNASLLKVYSKEDQDLLKLVKSYHWMGLVHIPTNGSWQWEDGSILSPNLLTII
EMQKGDCALYASSFKGYIENCSTPNTYICMQRTV
SEQ ID NO: 55. GPC3 scFv
DVVMTQSPLSLPVTPGEPASISCRSSQSLVHSNANTYLHWYLQKPGQSPQLLIYKVSNRFSG
VPDRFSGSGSGTDFTLKISRVEAEDVGVYYCSQNTHVPPTFGQGTKLEIKRGGGGSGGGGS
GGGGSQVQLVQSGAEVKKPGASVKVSCKASGYTFTDYEMHWVRQAPGQGLEWMGALDP
KTGDTAYSQKFKGRVTLTADESTSTAYMELSSLRSEDTAVYYCTRFYSYTYWGQGTLVTV
SS
SEQ ID NO: 56. Anti-GPC3 4-1BB CAR
MALPVTALLLPLALLLHAARPDYKDDDDKDVVMTQSPLSLPVTPGEPASISCRSSQSLVHS
NANTYLHWYLQKPGQSPQLLIYKVSNRFSGVPDRFSGSGSGTDFTLKISRVEAEDVGVYYC
SQNTHVPPTFGQGTKLEIKRGGGGSGGGGSGGGGSQVQLVQSGAEVKKPGASVKVSCKAS
GYTFTDYEMHWVRQAPGQGLEWMGALDPKTGDTAYSQKFKGRVTLTADESTSTAYMEL
SSLRSEDTAVYYCTRFYSYTYWGQGTLVTVSSTTTPAPRPPTPAPTIASQPLSLRPEACRPAA
GGAVHTRGLDFACDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDT
YDALHMQALPPR
SEQ ID NO: 57. Anti-GPC3 4-1BB CAR armored with NKG2D TLR4 chimeric receptor
MALPVTALLLPLALLLHAARPDYKDDDDKDVVMTQSPLSLPVTPGEPASISCRSSQSLVHS
NANTYLHWYLQKPGQSPQLLIYKVSNRFSGVPDRFSGSGSGTDFTLKISRVEAEDVGVYYC
SQNTHVPPTFGQGTKLEIKRGGGGSGGGGSGGGGSQVQLVQSGAEVKKPGASVKVSCKAS
GYTFTDYEMHWVRQAPGQGLEWMGALDPKTGDTAYSQKFKGRVTLTADESTSTAYMEL
SSLRSEDTAVYYCTRFYSYTYWGQGTLVTVSSTTTPAPRPPTPAPTIASQPLSLRPEACRPAA
GGAVHTRGLDFACDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDT
YDALHMQALPPRGSGATNFSLLKQAGDVEENPGPMALPVTALLLPLALLLHAARPSLENQ
EVQIPLTESYCGPCPKNWICYKNNCYQFFDESKNWYESQASCMSQNASLLKVYSKEDQDL
LKLVKSYHWMGLVHIPTNGSWQWEDGSILSPNLLTIIEMQKGDCALYASSFKGYIENCSTP
NTYICMQRTVTIIGVSVLSVLVVSVVAVLVYKFYFHLMLLAGCIKYGRGENIYDAFVIYSSQ
DEDWVRNELVKNLEEGVPPFQLCLHYRDFIPGVAIAANIIHEGFHKSRKVIVVVSQHFIQSR
WCIFEYEIAQTWQFLSSRAGIIFIVLQKVEKTLLRQQVELYRLLSRNTYLEWEDSVLGRHIF
WRRLRKALLDGKSWNPEGTVGTGCNWQEATSI
SEQ ID NO: 58. Anti-GPC3 4-1BB CAR armored with NKG2D CD8 TLR4 chimeric receptor
MALPVTALLLPLALLLHAARPDYKDDDDKDVVMTQSPLSLPVTPGEPASISCRSSQSLVHS
NANTYLHWYLQKPGQSPQLLIYKVSNRFSGVPDRFSGSGSGTDFTLKISRVEAEDVGVYYC
SQNTHVPPTFGQGTKLEIKRGGGGSGGGGSGGGGSQVQLVQSGAEVKKPGASVKVSCKAS
GYTFTDYEMHWVRQAPGQGLEWMGALDPKTGDTAYSQKFKGRVTLTADESTSTAYMEL
SSLRSEDTAVYYCTRFYSYTYWGQGTLVTVSSTTTPAPRPPTPAPTIASQPLSLRPEACRPAA
GGAVHTRGLDFACDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDT
YDALHMQALPPRGSGATNFSLLKQAGDVEENPGPMALPVTALLLPLALLLHAARPSLENQ
EVQIPLTESYCGPCPKNWICYKNNCYQFFDESKNWYESQASCMSQNASLLKVYSKEDQDL
LKLVKSYHWMGLVHIPTNGSWQWEDGSILSPNLLTIIEMQKGDCALYASSFKGYIENCSTP
NTYICMQRTVTTTPAPRPPTPAPTIASQPLSLRPEACRPAAGGAVHTRGLDFACDTIIGVSVL
SVLVVSVVAVLVYKFYFHLMLLAGCIKYGRGENIYDAFVIYSSQDEDWVRNELVKNLEEG
VPPFQLCLHYRDFIPGVAIAANIIHEGFHKSRKVIVVVSQHFIQSRWCIFEYEIAQTWQFLSSR
AGIIFIVLQKVEKTLLRQQVELYRLLSRNTYLEWEDSVLGRHIFWRRLRKALLDGKSWNPE
GTVGTGCNWQEATSI
SEQ ID NO: 59. Anti-CD19 4-1BB CAR armored with NKG2D TLR4 chimeric receptor
MALPVTALLLPLALLLHAARPDIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGGTK
LEITGGGGSGGGGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAIYYCAKHYYYG
GSYAMDYWGQGTSVTVSSTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQEEDGCSCRFPEEE
EGGCELRVKFSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
GSGATNFSLLKQAGDVEENPGPMALPVTALLLPLALLLHAARPSLFNQEVQIPLTESYCGPC
PKNWICYKNNCYQFFDESKNWYESQASCMSQNASLLKVYSKEDQDLLKLVKSYHWMGL
VHIPTNGSWQWEDGSILSPNLLTIIEMQKGDCALYASSFKGYIENCSTPNTYICMQRTVTIIG
VSVLSVLVVSVVAVLVYKFYFHLMLLAGCIKYGRGENIYDAFVIYSSQDEDWVRNELVKN
LEEGVPPFQLCLHYRDFIPGVAIAANIIHEGFHKSRKVIVVVSQHFIQSRWCIFEYEIAQTWQ
FLSSRAGIIFIVLQKVEKTLLRQQVELYRLLSRNTYLEWEDSVLGRHIFWRRLRKALLDGKS
WNPEGTVGTGCNWQEATSI
SEQ ID NO: 60. Anti-CD19 4-1BB CAR armored with NKG2D CD8 TLR4 chimeric receptor
MALPVTALLLPLALLLHAARPDIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGGTK
LEITGGGGSGGGGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAIYYCAKHYYYG
GSYAMDYWGQGTSVTVSSTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQEEDGCSCRFPEEE
EGGCELRVKFSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
GSGATNFSLLKQAGDVEENPGPMALPVTALLLPLALLLHAARPSLFNQEVQIPLTESYCGPC
PKNWICYKNNCYQFFDESKNWYESQASCMSQNASLLKVYSKEDQDLLKLVKSYHWMGL
VHIPTNGSWQWEDGSILSPNLLTIIEMQKGDCALYASSFKGYIENCSTPNTYICMQRTVTTTP
APRPPTPAPTIASQPLSLRPEACRPAAGGAVHTRGLDFACDTIIGVSVLSVLVVSVVAVLVY
KFYFHLMLLAGCIKYGRGENIYDAFVIYSSQDEDWVRNELVKNLEEGVPPFQLCLHYRDFI
PGVAIAANIIHEGFHKSRKVIVVVSQHFIQSRWCIFEYEIAQTWQFLSSRAGIIFIVLQKVEKT
LLRQQVELYRLLSRNTYLEWEDSVLGRHIFWRRLRKALLDGKSWNPEGTVGTGCNWQEA
TSI
SEQ ID NO: 61. Tandem anti-BCMA-co-anti-BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMALPVTALLLPLALLLHAARPAVQLVESGGGLV
QAGDSLRLTCTASGRAFSTYFMAWFRQAPGKEREFVAGIAWSGGSTAYADSVKGRFTISR
DNAKNTVYLQMNSLKSEDTAVYYCASRGIEVEEFGAWGQGTQVTVSSGGGGSQVQLEES
GGGSVQAGGSLRLSCAYTYSTYSNYYMGWFREAPGKARTSVAIISSDTTITYKDAVKGRFT
ISKDNAKNTLYLQMNSLKPEDSAMYRCAAWTSDWSVAYWGQGTQVTVSSTSQMNKTIIG
VSVLSVLVVSVVAVLVYKFYFHLMLLAGCIKYGRGENIYDAFVIYSSQDEDWVRNELVKN
LEEGVPPFQLCLHYRDFIPGVAIAANIIHEGFHKSRKVIVVVSQHFIQSRWCIFEYEIAQTWQ
FLSSRAGIIFIVLQKVEKTLLRQQVELYRLLSRNTYLEWEDSVLGRHIFWRRLRKALLDGKS
WNPEGTVGTGCNWQEATSI
SEQ ID NO: 62. Single anti-BCMA-co-anti-BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
RGSGATNFSLLKQAGDVEENPGPMALPVTALLLPLALLLHAARPQVQLEESGGGSVQAGG
SLRLSCAYTYSTYSNYYMGWFREAPGKARTSVAIISSDTTITYKDAVKGRFTISKDNAKNTL
YLQMNSLKPEDSAMYRCAAWTSDWSVAYWGQGTQVTVSSTSQMNKTIIGVSVLSVLVVS
VVAVLVYKFYFHLMLLAGCIKYGRGENIYDAFVIYSSQDEDWVRNELVKNLEEGVPPFQL
CLHYRDFIPGVAIAANIIHEGFHKSRKVIVVVSQHFIQSRWCIFEYEIAQTWQFLSSRAGIIFIV
LQKVEKTLLRQQVELYRLLSRNTYLEWEDSVLGRHIFWRRLRKALLDGKSWNPEGTVGTG
CNWQEATSI
SEQ ID NO: 63. Single anti-BCMA-co-anti-BCMA-CD8 CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
RGSGATNFSLLKQAGDVEENPGPMALPVTALLLPLALLLHAARPQVQLEESGGGSVQAGG
SLRLSCAYTYSTYSNYYMGWFREAPGKARTSVAIISSDTTITYKDAVKGRFTISKDNAKNTL
YLQMNSLKPEDSAMYRCAAWTSDWSVAYWGQGTQVTVSSTSTTTPAPRPPTPAPTIASQP
LSLRPEACRPAAGGAVHTRGLDFACDTIIGVSVLSVLVVSVVAVLVYKFYFHLMLLAGCIK
YGRGENIYDAFVIYSSQDEDWVRNELVKNLEEGVPPFQLCLHYRDFIPGVAIAANIIHEGFH
KSRKVIVVVSQHFIQSRWCIFEYEIAQTWQFLSSRAGIIFIVLQKVEKTLLRQQVELYRLLSR
NTYLEWEDSVLGRHIFWRRLRKALLDGKSWNPEGTVGTGCNWQEATSI
SEQ ID NO: 64. Single anti-BCMA-co-anti-BCMA-CD28 CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
RGSGATNFSLLKQAGDVEENPGPMALPVTALLLPLALLLHAARPQVQLEESGGGSVQAGG
SLRLSCAYTYSTYSNYYMGWFREAPGKARTSVAIISSDTTITYKDAVKGRFTISKDNAKNTL
YLQMNSLKPEDSAMYRCAAWTSDWSVAYWGQGTQVTVSSTSIEVMYPPPYLDNEKSNGT
IIHVKGKHLCPSPLFPGPSKPTIIGVSVLSVLVVSVVAVLVYKFYFHLMLLAGCIKYGRGENI
YDAFVIYSSQDEDWVRNELVKNLEEGVPPFQLCLHYRDFIPGVAIAANIIHEGFHKSRKVIV
VVSQHFIQSRWCIFEYEIAQTWQFLSSRAGIIFIVLQKVEKTLLRQQVELYRLLSRNTYLEWE
DSVLGRHIFWRRLRKALLDGKSWNPEGTVGTGCNWQEATSI
SEQ ID NO: 65. Tandem anti-BCMA-co-anti-GPRC5D CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMALPVTALLLPLALLLHAARPQSVVTQPPSMSAA
PGQQVTISCSGGNSNIERNYVSWYLQLPGTAPKLVIFDNDRRPSGIPDRFSGSKSGTSATLGI
TGLQTGDEADYYCGTWDSSLRGWVFGGGTKLTVLGSRGGGGSGGGGSGGGGSLEMAEV
QLVESGGGLIQPGGSLRLSCAASGFTFSNYAMNWVRQAPGKGLEWVSTINGRGSSTIYADS
VKGRFTISRDNSKNTLYLQMNSLRAEDTATYYCARYISRGLGDSWGQGTLVTVTIIGVSVL
SVLVVSVVAVLVYKFYFHLMLLAGCIKYGRGENIYDAFVIYSSQDEDWVRNELVKNLEEG
VPPFQLCLHYRDFIPGVAIAANIIHEGFHKSRKVIVVVSQHFIQSRWCIFEYEIAQTWQFLSSR
AGIIFIVLQKVEKTLLRQQVELYRLLSRNTYLEWEDSVLGRHIFWRRLRKALLDGKSWNPE
GTVGTGCNWQEATSI
SEQ ID NO: 66. Tandem anti-BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPR
SEQ ID NO: 67. Single anti-BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
R
SEQ ID NO: 68. First single anti-BCMA sdAb
AVQLVESGGGLVQAGDSLRLTCTASGRAFSTYFMAWFRQAPGKEREFVAGIAWSGGSTAY
ADSVKGRFTISRDNAKNTVYLQMNSLKSEDTAVYYCASRGIEVEEFGAWGQGTQVTVSS
SEQ ID NO: 69. Second single anti-BCMA sdAb
QVQLEESGGGSVQAGGSLRLSCAYTYSTYSNYYMGWFREAPGKARTSVAIISSDTTITYKD
AVKGRFTISKDNAKNTLYLQMNSLKPEDSAMYRCAAWTSDWSVAYWGQGTQVTVSS
SEQ ID NO: 70. Anti-GPRC5D scFv
QSVVTQPPSMSAAPGQQVTISCSGGNSNIERNYVSWYLQLPGTAPKLVIFDNDRRPSGIPDR
FSGSKSGTSATLGITGLQTGDEADYYCGTWDSSLRGWVFGGGTKLTVLGSRGGGGSGGGG
SGGGGSLEMAEVQLVESGGGLIQPGGSLRLSCAASGFTFSNYAMNWVRQAPGKGLEWVST
INGRGSSTIYADSVKGRFTISRDNSKNTLYLQMNSLRAEDTATYYCARYISRGLGDSWGQG
TLVTV
SEQ ID NO: 71. CLL1-co-CD33-2 CAR
MALPVTALLLPLALLLHAARPQVQLVESGGGSVQAGGALSLSCAASGYTVRIDYMGWYR
QTPGKGREPVATIASNGGTAYADSVEGRFTISQDNAKNSVYLQMNTLKPGDTAMYYCAA
GTWPTLTYFGQGTQVTVSSTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
GSGATNFSLLKQAGDVEENPGPMALPVTALLLPLALLLHAARPEVQLVESGGGSVQAGGS
LRLSCAASGYTYSINCMGWFRQAPGKEREGVAVISTGGGRTDYRDSVKGRFTISQDNAKN
TVYLQMNSLKPEDTAMYYCAGKTTYPGYGCGLGRSAYNYWGQGTQVTVSSQMNKTIIGV
SVLSVLVVSVVAVLVYKFYFHLMLLAGCIKYGRGENIYDAFVIYSSQDEDWVRNELVKNL
EEGVPPFQLCLHYRDFIPGVAIAANIIHEGFHKSRKVIVVVSQHFIQSRWCIFEYEIAQTWQF
LSSRAGIIFIVLQKVEKTLLRQQVELYRLLSRNTYLEWEDSVLGRHIFWRRLRKALLDGKS
WNPEGTVGTGCNWQEATSI
SEQ ID NO: 72. Tandem anti-BCMA-co-anti-GPRC5D-CD8 CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMALPVTALLLPLALLLHAARPQSVVTQPPSMSAA
PGQQVTISCSGGNSNIERNYVSWYLQLPGTAPKLVIFDNDRRPSGIPDRFSGSKSGTSATLGI
TGLQTGDEADYYCGTWDSSLRGWVFGGGTKLTVLGSRGGGGSGGGGSGGGGSLEMAEV
QLVESGGGLIQPGGSLRLSCAASGFTFSNYAMNWVRQAPGKGLEWVSTINGRGSSTIYADS
VKGRFTISRDNSKNTLYLQMNSLRAEDTATYYCARYISRGLGDSWGQGTLVTVTTTPAPRP
PTPAPTIASQPLSLRPEACRPAAGGAVHTRGLDFACDTTIIGVSVLSVLVVSVVAVLVYKFY
FHLMLLAGCIKYGRGENIYDAFVIYSSQDEDWVRNELVKNLEEGVPPFQLCLHYRDFIPGV
AIAANIIHEGFHKSRKVIVVVSQHFIQSRWCIFEYEIAQTWQFLSSRAGIIFIVLQKVEKTLLR
QQVELYRLLSRNTYLEWEDSVLGRHIFWRRLRKALLDGKSWNPEGTVGTGCNWQEATSI
SEQ ID NO: 73. Tandem anti-BCMA-co-anti-GPRC5D-CD28 CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMALPVTALLLPLALLLHAARPQSVVTQPPSMSAA
PGQQVTISCSGGNSNIERNYVSWYLQLPGTAPKLVIFDNDRRPSGIPDRFSGSKSGTSATLGI
TGLQTGDEADYYCGTWDSSLRGWVFGGGTKLTVLGSRGGGGSGGGGSGGGGSLEMAEV
QLVESGGGLIQPGGSLRLSCAASGFTFSNYAMNWVRQAPGKGLEWVSTINGRGSSTIYADS
VKGRFTISRDNSKNTLYLQMNSLRAEDTATYYCARYISRGLGDSWGQGTLVTVIEVMYPPP
YLDNEKSNGTIIHVKGKHLCPSPLFPGPSKPTIIGVSVLSVLVVSVVAVLVYKFYFHLMLLA
GCIKYGRGENIYDAFVIYSSQDEDWVRNELVKNLEEGVPPFQLCLHYRDFIPGVAIAANIIH
EGFHKSRKVIVVVSQHFIQSRWCIFEYEIAQTWQFLSSRAGIIFIVLQKVEKTLLRQQVELYR
LLSRNTYLEWEDSVLGRHIFWRRLRKALLDGKSWNPEGTVGTGCNWQEATSI

What is claimed is:

1. A modified immune cell comprising:
a) a first polypeptide comprising: i) a first target binding domain, ii) a first TLR transmembrane domain, and iii) a first TLR signaling domain; and
b) a second polypeptide comprising: i) a second target binding domain, ii) a second TLR transmembrane domain, and iii) a second TLR signaling domain;
wherein upon binding of the first target binding domain and second target binding domain to their corresponding target, the first TLR signaling domain and the second TLR signaling domain associate with each other to form a TLR signaling moiety capable of inducing TLR signaling.

2. The modified immune cell of claim 1, wherein the first target binding domain and the second target binding domain each binds to a subunit of a multimeric target molecule.

3. The modified immune cell of claim 2, wherein the subunits of the multimeric target molecule are the same.

4. The modified immune cell of claim 2, wherein the subunits of the multimeric target molecule are different.

5. The modified immune cell of claim 1 or 2, wherein the first target binding domain and the second binding domain bind to the same target molecule.

6. The modified immune cell of claim 5, wherein the first target binding domain and the second binding domain each binds to the same target site on the target molecule.

7. The modified immune cell of any one of claims 1-3, and 5-6, wherein the first target binding domain and the second target binding domain are the same.

8. The modified immune cell of claim 5, wherein the first target binding domain and the second target binding domain each binds to a different non-overlapping target sites on a single target molecule.

9. The modified immune cell of any one of claims 1-8, wherein the first TLR transmembrane domain and the first TLR signaling domain are derived from the same TLR molecule.

10. The modified immune cell of any one of claims 1-9, wherein the second TLR transmembrane domain and the second TLR signaling domain are derived from the same TLR molecule.

11. The modified immune cell of any one of claims 1-10, wherein the first TLR transmembrane domain and the second TLR transmembrane domain are the same.

12. The modified immune cell of any one of claims 1-11, wherein the first TLR signaling domain and the second TLR signaling domain are the same.

13. The modified immune cell of claim 11 or claim 12, wherein the first TLR transmembrane domain and/or first TLR signaling domain are derived from TLR4.

14. The modified immune cell of any one of claims 1-10, wherein the first TLR transmembrane domain and the second TLR transmembrane domain are different.

15. The modified immune cell of any one of claims 1-11 and 14, wherein the first TLR signaling domain and the second TLR signaling domain are different.

16. The modified immune cell of claim 14 or claim 15, wherein the first TLR transmembrane domain and/or first TLR signaling domain are derived from TLR2.

17. The modified immune cell of claim 14 or claim 15, wherein the second TLR transmembrane domain and/or second TLR signaling domain are derived from TLR6.

18. The modified immune cell of any one of claims 14-17, wherein the second TLR transmembrane domain and/or second TLR signaling domain are derived from TLR1.

19. The modified immune cell of any one of claims 1-18, wherein the first target binding domain and/or the second target binding domain is an antibody moiety or antigen-binding fragment thereof.

20. The modified immune cell of claim 19, wherein the first target binding domain and/or second target binding domain is a scFv or sdAb.

21. The modified immune cell of claim 20, wherein the scFv or sdAb specifically binds to CD33, CLL1, CD123, CD19, CD20, CD22, BCMA, GPRC5D, and GPC3.

22. The modified immune cell of any one of claims 5-18, wherein the target molecule is an immune checkpoint protein.

23. The modified immune cell of claim 22, wherein the target molecule is selected from the group consisting of PD-1, CD70, CD27, SIRPα, and TIGIT.

24. The modified immune cell of any one of claims 5-18, wherein the target molecule is a natural protein expressed on immune cells.

25. The modified immune cell of claim 24, wherein the target molecule is NKG2D.

26. The modified immune cell of claim 24 or 25, wherein the target molecule is a full-length sequence of NKG2D.

27. The modified immune cell of claim 24 or 25, wherein the target molecule is mutated NKG2D.

28. The modified immune cell of claim 27, wherein the mutated NKG2D comprises a truncated sequence, and/or an amino acid a substitution, mutation, addition, and/or deletion.

29. The modified immune cell of claim 24 or 25, wherein the target molecule is an extracellular antigen binding domain of NKG2D.

30. The modified immune cell of any one of claims 1-29, wherein the modified immune cell is selected from the group consisting of a cytotoxic T cell, a helper T cell, a natural killer (NK) cell, an NK-cell, an iNK-T cell, an NK-T like cell, an αβT cell and a γδT cell.

31. The modified immune cell of claim 30, wherein the modified immune cell is an NK cell.

32. The modified immune cell of claim 30, wherein the modified immune cell is a cytotoxic T cell.

33. The modified immune cell of any one of claims 1-32, wherein the modified immune cell comprises an engineered receptor.

34. The modified immune cell of claim 33, wherein the engineered receptor is a chimeric antigen receptor (CAR).

35. The modified immune cell of claim 33, wherein the engineered receptor is a modified T-cell receptor (TCR).

36. The modified immune cell of claim 33, wherein the engineered receptor is a T-cell antigen coupler (TAC) receptor.

37. The modified immune cell of any one of claims 33-36, wherein the engineered receptor comprises an extracellular domain specifically recognizing the same target molecule as the first polypeptide and/or the second polypeptide.

38. The modified immune cell of claim 37, wherein the engineered receptor comprises an extracellular domain specifically recognizing a non-overlapping target site on the same target molecule as the first polypeptide and/or the second polypeptide.

39. The modified immune cell of any one of claims 33-38, wherein the engineered receptor comprises an extracellular domain specifically recognizing any of CD19, CLL1, BCMA, and GPC3.

40. The modified immune cell of claim 39, wherein the engineered receptor comprises an amino acid sequence having at least about 95% sequence identity to the amino acid sequence of any one of SEQ ID NOs: 1, 3, 57-60, 61-65, 71-73 or comprises an amino acid sequence of any one of SEQ ID NOs: 1, 3, 57-60, 61-65, 71-73.

41. The modified immune cell of any one of claims 1-40, wherein the modified immune cell comprises a first nucleic acid encoding the first polypeptide and a second nucleic acid encoding the second polypeptide.

42. The modified immune cell of any one of claims 1-3, 5-7, 9-13, and 19-32, wherein the first polypeptide and the second polypeptide are the same, and wherein the modified immune cell comprises a first nucleic acid encoding the first polypeptide and the second polypeptide.

43. The modified immune cell of any one of claims 33-42, wherein the modified immune cell comprises a third nucleic acid encoding the engineered receptor.

44. The modified immune cell of any one of claims 41-43, wherein the first nucleic acid and the second nucleic acid are operably linked to the same promoter.

45. The modified immune cell of claim 41 or 43, wherein the first nucleic acid and the second nucleic acid are operably linked to separate promoters.

46. The modified immune cell of any one of claims 43-45, wherein the first nucleic acid and the third nucleic acid are operably linked to the same promoter.

47. The modified immune cell of any one of claims 43-45, wherein the first nucleic acid and the third nucleic acid are operably linked to separate promoters.

48. The modified immune cell of any one of claims 43, 44, and 46, wherein the first nucleic acid, the second nucleic acid, and the third nucleic acid are operably linked to the same promoter.

49. The modified immune cell of any one of claims 19-21, wherein the first target binding domain and second target binding domain specifically recognize a subunit of CD20.

50. The modified immune cell of any one of claims 8-48, (1) the first target binding domain specifically recognizes the C2 subunit of CD33, and the second target binding domain specifically recognizes the V subunit of CD33; or
(2) the first target binding domain specifically recognizes the V subunit of CD33, and the second target binding domain specifically recognizes the C2 subunit of CD33.

51. The modified immune cell of any one of claims 1-50, wherein the first polypeptide further comprises a first intracellular domain of a first cytokine receptor, and/or the second polypeptide further comprises a second intracellular domain of a second cytokine receptor.

52. The modified immune cell of claim 51, wherein the first intracellular domain and the second intracellular domain are the same.

53. The modified immune cell of claim 51, wherein the first intracellular domain and the second intracellular domain are different.

54. The modified immune cell of any of claims 51-53, wherein the first cytokine receptor and/or the second cytokine receptor is selected from the group consisting of a GM-CSF receptor, an IL-18 receptor, an IL-21 receptor, an IL-15 receptor, and an IL-23 receptor.

55. The modified immune cell of any of claims 51-53, wherein the first intracellular domain of the first cytokine receptor and/or the second intracellular domain of the second cytokine receptor comprise an immunoreceptor tyrosine-based activation motif (ITAM).

56. The modified immune cell of any one of claims 51-55, wherein the C-terminus of the first intracellular domain of the first cytokine receptor is fused to the N-terminus of the first TLR signaling domain; and/or the C-terminus of the second intracellular domain of the second cytokine receptor is fused to the N-terminus of the second TLR signaling domain.

57. The modified immune cell of any one of claims 51-55, wherein the N-terminus of the first intracellular domain of the first cytokine receptor is fused to the C-terminus of the TLR signaling domain, and/or the N-terminus of the second intracellular domain of the second cytokine receptor is fused to the C-terminus of the TLR signaling domain.

58. A method of producing a modified immune cell of any one of claims 1-57, comprising:
introducing into a precursor immune cell a first nucleic acid encoding the first polypeptide and optionally a second nucleic acid encoding the second polypeptide.

59. The method of claim 58, wherein the precursor immune cell is selected from the group consisting of a cytotoxic T cell, a helper T cell, an NK cell, an NK-T cell, an iNK-T cell, an NK-T like cell, an αβT cell and a γδT cell.

60. The method of claim 58 or 59, wherein the precursor immune cell comprises an engineered receptor.

61. The method of claim 58 or 59, further comprising introducing into the precursor immune cell a third nucleic acid encoding an engineered receptor.

62. The method of claim 60 or 61, wherein the engineered receptor is a chimeric antigen receptor (CAR), a modified T-cell receptor (TCR), or a T-cell antigen coupler (TAC) receptor.

63. The method of any one of claims 58-62, wherein the first nucleic acid, the second nucleic acid, and/or the third nucleic acid are on the same vector.

64. The method of claim 63, wherein the vector is a viral vector.

65. The method of claim 64, wherein the viral vector is selected from the group consisting of an adenoviral vector, an adeno-associated virus vector, a retroviral vector, a lentiviral vector, a herpes simplex viral vector, and derivatives thereof.

66. The method of any one of claims 58-62, further comprising isolating or enriching immune cells comprising the first nucleic acid, the second nucleic acid, and/or the third nucleic acid.

67. A modified immune cell produced by the method of any one of claims 58-66.

68. A pharmaceutical composition comprising the modified immune cell of claims 1-57 and 67, and a pharmaceutically acceptable carrier.

69. A method of treating a disease in an individual, comprising administering to the individual an effective amount of the pharmaceutical composition of claim 68.

70. The method of claim 69, wherein the disease is cancer.

71. The method of claim 69 or 70, wherein the individual is human.
